(19) United States
(12) Patent Application Publication
(10) Pub. No.: US 2008/0090863 A1
(43) Pub. Date: Apr. 17, 2008

(54) Pyridine Derivatives and Their Use as Medicaments for Treating Diseases Related to Mch Receptor

(75) Inventors: Yoshinori Sekiguchi, Tokyo (JP); Kosuke Kanuma, Tokyo (JP); Katsunori Omodera, Tokyo (JP); Kumi Sato, Tokyo (JP); Masato Hayashi, Tokyo (JP); Shuji Yamamoto, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: TAISHO PHARMACEUTICAL CO., LTD., Tokyo (JP); ARENA PHARMACEUTICALS, San Deigo, CA (US)

(21) Appl. No.: 11/664,392

(22) PCT Filed: Sep. 27, 2005
(86) PCT No.: PCT/JP05/18237

§ 371 Date: Jul. 30, 2007

Related U.S. Application Data
(60) Provisional application No. 60/614,358, filed on Sep. 30, 2004.

(51) Int. Cl. A61K 31/44; A61K 31/4439; A61K 31/47; A61P 25/00; A61P 3/04; A61P 9/00; C07D 213/75; C07D 215/00; C07D 401/08; C07D 413/08

(52) U.S. Cl. 514/311; 514/338; 514/343; 514/352; 546/152; 546/269.1; 546/276.4; 546/304

(57) ABSTRACT

The present invention encompasses novel substituted pyridine compounds of Formula (I): 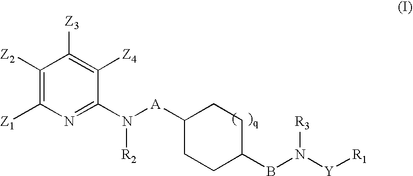 which act as MCH receptor antagonists. These compositions and pharmaceutical compositions thereof are useful in the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction.

### FIELD OF THE INVENTION

The present invention relates to compounds which act as antagonists for MCH receptors and to the use of these compounds in pharmaceutical compositions.

### BACKGROUND OF THE INVENTION

Melanin Concentrating Hormone (MCH), a cyclic peptide, has been identified as the endogenous ligand of the orphan G-protein coupled receptor SLC-1. See, for example, Shimomura et al., Biochem. Biophys. Res. Commun. 261, 622-26 (1999). Studies have indicated that MCH acts as a neurotransmitter/neuromodulator to alter a number of behavioral responses such as feeding habits. For example, injection of MCH into rats has been reported to increase their consumption of food. Reports indicate that genetically engineered mice which lack MCH show lower body weight and increased metabolism. See Saito et al., TEM, vol. 11, 299 (2000). As such, the literature suggests that discovery of MCH antagonists that interact with SCL-1 expressing cells will be useful in developing obesity treatments. See Shimomura et al., Biochem. Biophys. Res. Commun. 261, 622-26 (1999).

G protein-coupled receptors (GPCRs) share a common structural motif. All these receptors have seven sequences of between 22 to 24 hydrophobic amino acids that form seven alpha helices, each of which spans the membrane. The fourth and fifth transmembrane helices are joined on the extracellular side of the membrane by a strand of amino acids that forms a relatively large loop. Another larger loop, composed primarily of hydrophilic amino acids, joins transmembrane helices five and six on the intracellular side of the membrane. The carboxy terminus of the receptor lies intracellularly, and the amino terminus lies in the extracellular space. It is thought that the loop joining helices five and six, as well as the carboxy terminus, interact with the G protein. Currently, Gq, Gs, Gi, and Go are G proteins that have been identified as possible proteins that interact with the receptor.

Under physiological conditions, GPCRs exist in the cell membrane in equilibrium between two different states or conformations: an “inactive” state and an “active” state. A receptor in an inactive state is unable to link to the intracellular transduction pathway to produce a biological response. Changing the receptor conformation to the active state allows linkage to the transduction pathway and produces a biological response.

A receptor may be stabilized in an active state by an endogenous ligand or an exogenous agonist ligand. Recent discoveries, including but not exclusively limited to, modifications to the amino acid sequence of the receptor, provide alternative mechanisms other than ligands to stabilize the active state conformation. These approaches effectively stabilize the receptor in an active state by simulating the effect of a ligand binding to the receptor. Stabilization by such ligand-independent approaches is termed “constitutive receptor activation.” In contrast, antagonists can competitively bind to the receptor at the same site as agonists, but do not activate the intracellular response initiated by the active form of the receptor, and therefore inhibit the intracellular responses by agonists.

Certain 2-aminoquinazoline derivatives have been reported to be NPY antagonists which are reported to be effective in the treatment of disorders and diseases associated with the NPY receptor subtype Y5. See PCT Patent Application 97/20823. Quinazoline derivatives have also been found to be useful by enhancing antitumor activity. See PCT Patent Application 92/07844. And also the quinoline derivatives which have an antagonist activity for MCH receptor are known in these patents, WO03/070244, WO03/105850, WO03/45313, WO03/045920, and WO04/04726.

Recently, our current knowledge of human obesity has advanced dramatically. Previously, obesity was viewed as an oppugnant behavior of inappropriate eating in the setting of appealing foods. Studies of animal models of obesity, biochemical alterations in both humans and animals, and the complex interactions of psychosocial and cultural factors that create receptiveness to human obesity indicate that this disease in humans is multifaceted and deeply entrenched in biologic systems. Thus, it is almost certain that obesity has multiple causes and that there are different types of obesity. Not only does MCHR1 antagonist have potent and durable anti-obesity effects in rodents, it has surprising antidepressant and anxiolytic properties as well (Borowsky et al., Nature Medicine, 8, 825-830, 2002). MCHR1 antagonists have been reported to show antidepressant and anxiolytic activities in rodent models such as social interaction, forced swimming test and ultrasonic vocalization. These findings indicate that MCHR1 antagonists could be useful for treatment of obesity patients with multiple causes. Moreover, MCHR1 antagonists could be used to treat subjects not only with obesity, but also those with depression and anxiety. These advantages make it different from NPY receptor antagonists, with which anxiogenic-like activity can be expected, as NPY itself has anxiolytic-like effect.

Obesity is also regarded as a chronic disease and the possibly of long-term treatment is a concept that is receiving more attention. In this context, it is noteworthy that the depletion of MCH leads to hypophagia as well as leanness (Shimada et al., Nature, 396, 670-674, 1998). By contrast, NPY (Erickson et al., Nature, 381, 415-418, 1996), as well as the Y1 (Pedrazzini et al., Nature Medicine, 4, 722-726, 1998) and Y5 receptors (Marsh et al., Nature Medicine, 4, 718-721, 1998), disrupted mice maintained a stable body weight or rather became obese. Considering the above reports, MCHR1 antagonists can be more attractive than Y1 or Y5 receptor antagonists in terms of long-term treatment of obese patients.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increase insulin secretion balances insulin resistance and protects patients from hyperglycemia (Le Stunff, et al. Diabetes 43, 696-702 (1989)). However, after several decades, β cell function deteriorates and non-insulin-dependent diabetes develops in about 20% of the obese population (Pederson, P. Diab. Metab. Rev. 5, 505-509 (1989)) and (Brancati, F. L., et al., Arch. Intern. Med. 159, 957-963 (1999)). Given its high prevalence in modern societies, obesity has thus become the leading risk factor for NIDDM (Hill, J. O., et al., Science 280, 1371-1374 (1998)). However, the factors which predispose a fraction of patients to alteration of insulin secretion in response to fat accumulation remain unknown.

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see TABLE below). There are problems with this definition in that it does not take into account the proportion of body mass that is muscle in relation to fat (adipose tissue). To account for this, obesity can also be defined on the basis of body fat content: greater than 25% and 30% in males and females, respectively.

CLASSIFICATION OF WEIGHT
BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Compounds marketed as anti-obesity agents include Orlistat (XENICAL™) and Sibutramine. Orlistat (a lipase inhibitor) inhibits fat absorption directly and tends to produce a high incidence of unpleasant (though relatively harmless) side-effects such as diarrhea. Sibutramine (a mixed 5-HT/noradrenaline reuptake inhibitor) can increase blood pressure and heart rate in some patients. The serotonin releaser/reuptake inhibitors fenfluramine (Pondimin™) and dexfenfluramine (Redux™) have been reported to decrease food intake and body weight over a prolonged period (greater than 6 months). However, both products were withdrawn after reports of preliminary evidence of heart valve abnormalities associated with their use. Accordingly, there is a need for the development of a safer anti-obesity agent.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of these conditions based on the prevention of obesity (Perry, I. J., et al., BMJ 310, 560-564 (1995)).

An increasing number of children and adolescents are overweight. Although not all overweight children will necessarily become overweight adults, the growing occurrence of obesity in childhood is likely to be reflected in increasing obesity in adult years. The high prevalence of obesity in our adult population and the likelihood that the nation of the future will be even more obese demands a re-examination of the health implications of this disease. See, Health Implications of Obesity. NIH Consens. Statement Online 1985 Feb. 11-13; 5(9):1-7.

“Clinical obesity” is a measurement of the excess body fat relative to lean body mass and is defined as a body weight more than 20% above the ideal body weight. Recent estimates suggest that 1 in 2 adults in the United States is clinically obese, an increase of more than 25% over the past decades. Flegal M. D. et al., 22 Int. J. Obes. Relat. Metab. Disor. 39 (1998). Both overweight conditions and clinical obesity are a major health concerns worldwide, in particular because clinical obesity is often accompanied by numerous complications, i.e., hypertension and Type II diabetes, which in turn can cause coronary artery disease, stroke, late-stage complications of diabetes and premature death. (See, e.g., Nishina P. M. et al., 43 Metab. 554 (1994)).

Although the etiologic mechanisms underlying obesity require further clarification, the net effect of such mechanisms leads to an imbalance between energy intake and expenditure. Both genetic and environmental factors are likely to be involved in the pathogenesis of obesity. These include excess caloric intake, decreased physical activity, and metabolic and endocrine abnormalities.

Treatment of overweight conditions and clinical obesity via pharmaceutical agents are not only of importance with respect to the conditions themselves, but also with respect to the possibility of preventing other diseases that are associated with, e.g., clinical obesity, as well as enhancement of the positive feeling of “self” that often accompanies those who are overweight or clinically obese and who encounter a significant reduction in body weight. Given the foregoing discussion, it is apparent that compounds which help in the treatment of such disorders would be useful and would provide an advance in both research and clinical medicine. The present invention is directed to these, as well as other, important ends.

### SUMMARY OF THE INVENTION

The present invention is drawn to compounds, which bind to and modulate the activity of a GPCR referred to herein as MCH, and uses thereof. The term MCH, as used herein, includes the human sequences found in GeneBank accession number NM_{—}005297, naturally-occurring allelic variants, mammalian orthologs, biologically active fragments and recombinant mutants thereof.

One aspect of the present invention relates to certain substituted pyridine compounds represented by Formula (I): 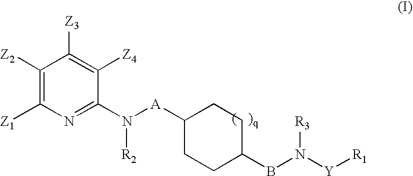
R₁ is selected from the group consisting of:
(i) C₁₋₁₀ alkyl, and
   C₁₋₁₀ alkyl substituted by substituent(s) independently selected from the group consisting of:
      halogen,
      oxo,
      C₁₋₅ alkoxy,
      C₁₋₅ alkoxy substituted by carbocyclic aryl,
      C₁₋₅ alkylcarbonyloxy,
      carbocyclic aryloxy,
      carbocyclic aryloxy substituted by substituent(s) independently selected from the group consisting of:
         halogen,
         nitro,
         C₁₋₅ alkyl, and
         C₁₋₅ alkyl substituted by oxo,
      heterocyclyloxy,
      heterocyclyloxy substituted by C₁₋₅ alkyl,
      C₁₋₅ alkoxycarbonyl,
      C₁₋₅ alkoxycarbonyl substituted by carbocyclic aryl,
      mono-carbocyclic arylamino,
      mono-carbocyclic arylamino substituted by hydroxy,
      di-carbocyclic arylamino,
      di-carbocyclic arylamino substituted by hydroxy,
      C₁₋₅ alkylcarbonylamino,
      heterocyclyl carbonylamino,
      carbocyclic arylsulfonylamino,
      carbocyclic arylsulfonylamino substituted by nitro,
      carbocyclic arylsulfonylamino substituted by C₁₋₅ alkyl,
      C₁₋₅ alkylthio,
      C₁₋₅ alkylthio substituted by carbocyclic aryl,
      carbocyclic arylthio,
      carbocyclic arylthio substituted by halogen,
      carbocyclic arylthio substituted by C₁₋₅ alkyl,
      carbocyclic arylsulfonyl,
      carbocyclic arylsulfonyl substituted by halogen,
      heterocyclylthio,
      heterocyclylthio substituted by C₁₋₅ alkyl,
      C₃₋₆ cycloalkyl,
      C₃₋₆ cycloalkenyl,
      carbocyclyl,
      carbocyclyl substituted by C₁₋₅ alkoxy,
      carbocyclic aryl,
      carbocyclic aryl substituted by substituent(s) independently selected from the group consisting of:
         halogen,
         hydroxy,
         nitro,
         C₁₋₅ alkyl,
         C₁₋₅ alkyl substituted by oxo,
         C₁₋₅ alkyl substituted by carbocyclic aryl,
         C₁₋₅ alkyl substituted by heterocyclyl,
         C₁₋₅ alkoxy,
         C₁₋₅ alkoxy substituted by halogen,
         C₁₋₅ alkoxy substituted by carbocyclic aryl,
         carbocyclic aryloxy,
         mono-carbocyclic arylamino,
         mono-carbocyclic arylamino substituted by halogen,
         di-carbocyclic arylamino,
         di-carbocyclic arylamino substituted by halogen,
         mono-carbocyclic arylaminocarbonyl,
         mono-carbocyclic arylaminocarbonyl substituted by substituent(s) selected from the group consisting of:
         halogen,
         C₁₋₅ alkyl,
         C₁₋₅ alkoxy, and
         C₁₋₅ alkoxy substituted by halogen,
         di-carbocyclic arylaminocarbonyl,
         di-carbocyclic arylaminocarbonyl substituted by substituent(s) selected from the group consisting of:
         halogen,
         C₁₋₅ alkyl,
         C₁₋₅ alkoxy, and
         C₁₋₅ alkoxy substituted by halogen,
         mercapto,
         C₁₋₅ alkylthio,
         C₁₋₅ alkylthio substituted by halogen,
         C₁₋₅ alkylsulfonyl,
         carbocyclic aryl, and
         heterocyclyl,
      heterocyclyl, and
      heterocyclyl substituted by substituent(s) independently selected from the group consisting of:
         C₁₋₅ alkyl,
         C₁₋₅ alkoxy,
         C₁₋₅ alkoxy substituted by carbocyclic aryl,
         carbocyclic aryl, and
         carbocyclic aryl substituted by halogen,
(ii) C₂₋₅ alkenyl, and
   C₂₋₅ alkenyl substituted by substituent(s) independently selected from the group consisting of:
      oxo,
      carbocyclic aryl,
      carbocyclic aryl substituted by substituent(s) independently selected from the group consisting of:
         halogen,
         nitro,
         C₁₋₅ alkyl,
         C₁₋₅ alkyl substituted by halogen,
         C₁₋₅ alkoxy, and
         C₁₋₅ alkoxy substituted by halogen,
(iii) C₃₋₆ cycloalkyl, and
   C₃₋₆ cycloalkyl substituted by substituent(s) independently selected from the group consisting of:
      C₁₋₅ alkyl,
      C₁₋₅ alkyl substituted by oxo,
      C₁₋₅ alkyl substituted by carbocyclic aryl,
      carbocyclic arylcarbonylamino, and
      carbocyclic aryl, and
      carbocyclic aryl by halogen,
(iv) carbocyclyl, and
   carbocyclyl substituted by nitro,
(v) carbocyclic aryl, and
   carbocyclic aryl substituted by substituent(s) independently selected from the group consisting of:
      halogen,
      hydroxy,
      cyano,
      nitro,
      C₁₋₉ alkyl,
      C₁₋₉ alkyl substituted by substituent(s) independently selected from the group consisting of:
         oxo,
         halogen,
         carbocyclic aryloxy,
         mono-carbocyclic arylaminocarbonyl,
         di-carbocyclic arylaminocarbonyl,
         mono-carbocyclic arylaminocarbonyl substituted by C₁₋₅ alkoxy,
         di-carbocyclic arylaminocarbonyl substituted by C₁₋₅ alkoxy,
         carbocyclic aryl,
         carbocyclic aryl substituted by substituent(s) independently selected from the group consisting of:
         halogen,
         C₁₋₅ alkyl, and
         C₁₋₅ alkyl substituted by halogen,
         heterocyclyl, and
         heterocyclyl substituted by C₁₋₅ alkyl,
      C₁₋₇ alkoxy,
      C₁₋₇ alkoxy substituted by halogen,
      C₁₋₇ alkoxy substituted by carbocyclic aryl,
      C₁₋₅ alkylcarbonyloxy,
      carbocyclic aryloxy,
      carbocyclic aryloxy substituted by C₁₋₅ alkoxy,
      C₁₋₅ alkoxycarbonyl,
      mono-C₁₋₅ alkylaminocarbonyl,
      di-C₁₋₅ alkylaminocarbonyl,
      mono-C₁₋₅ alkylaminocarbonyl substituted by carbocyclic aryl,
      di-C₁₋₅ alkylaminocarbonyl substituted by carbocyclic aryl,
      mono-carbocyclic arylaminocarbonyl,
      di-carbocyclic arylaminocarbonyl,
      mono-carbocyclic arylaminocarbonyl substituted by C₁₋₅ alkyl,
      di-carbocyclic arylaminocarbonyl substituted by C₁₋₅ alkyl,
      mono-C₁₋₅ alkylamino,
      di-C₁₋₅ alkylamino,
      carbocyclic arylsulfonylamino,
      carbocyclic arylsulfonylamino substituted by C₁₋₅ alkyl,
      C₁₋₅ alkylthio,
      C₁₋₅ alkylthio substituted by halogen,
      carbocyclic arylthio,
      carbocyclic arylthio substituted by cyano,
      C₁₋₅ alkylsulfonyl,
      mono-C₁₋₅ alkylaminosulfonyl,
      di-C₁₋₅ alkylaminosulfonyl,
      carbocyclic aryl,
      carbocyclic aryl substituted by substituent(s) independently selected from the group consisting of:
         C₁₋₇ alkyl, and
         C₁₋₇ alkyl substituted by halogen,
      heterocyclyl,
(vi) heterocyclyl, and
   heterocyclyl substituted by substituent(s) independently selected from the group consisting of:
      halogen,
      nitro,
      C₁₋₅ alkyl,
      C₁₋₅ alkyl substituted by substituent(s) independently selected from the group consisting of:
         halogen,
         oxo,
         carbocyclic aryl,
         carbocyclic aryl substituted by halogen, and
         heterocyclyl,
      C₁₋₅ alkoxy,
      carbocyclic aryloxy,
      carbocyclic aryloxy substituted by C₁₋₅ alkyl,
      C₁₋₅ alkylthio,
      carbocyclic arylthio,
      C₁₋₅ alkylsulfonyl,
      carbocyclic arylsulfonyl,
      carbocyclic arylsulfonyl substituted by halogen,
      carbocyclic arylsulfonyl substituted by C₁₋₅ alkyl,
      carbocyclic aryl,
      carbocyclic aryl substituted by substituent(s) independently selected from the group consisting of:
         halogen,
         nitro, and
         C₁₋₅ alkyl,
      heterocyclyl, and
      heterocyclyl substituted by substituent(s) independently selected from the group consisting of:
         C₁₋₅ alkyl, and
         C₁₋₅ alkyl substituted by halogen;
R₂ and R₃ are each independently hydrogen or C₁₋₅ alkyl; and A and B are each independently a single bond, —CH₂—, or —(CH₂)₂—;
Z₁, Z₂, Z₃, and Z₄ are each independently hydrogen, halogen, cyano, nitro, carboxy, carbamoyl, C₁₋₅ alkyl, C₁₋₅ alkyl substituted by halogen, C₁₋₅ alkyl substituted by hydroxy, C₁₋₅ alkoxy, C₁₋₅ alkoxy substituted by halogen, C₁₋₅ alkoxy substituted by hydroxy, —CO₂R₄ₐ, —C(O)N(R₄ₐ)(R_{4b}), —N(R₄ₐ)(R_{4b}), or heterocyclyl; wherein R₄ₐ and R_{4b} are each independently hydrogen, C₁₋₅ alkyl, or C₁₋₅ alkyl substituted by substituent(s) independently selected from the group consisting of:
   halogen,
   hydroxy,
   carboxy,
   carbamoyl,
   C₁₋₅ alkoxy,
   amino,
   C₃₋₆ cycloalkyl,
   carbocyclic aryl,
   carbocyclic aryl substituted by substituent(s) independently selected from the group consisting of:
      halogen,
      C₁₋₅ alkyl,
      C₁₋₅ alkyl substituted by halogen,
      C₁₋₅ alkoxy,
      C₁₋₅ alkoxy substituted by halogen, and
      —SO₂NH₂,
   heterocyclyl, and
C₃₋₆ cycloalkyl, carbocyclic aryl, carbocyclic aryl substituted by substituent(s) independently selected from the group consisting of:
   halogen,
   C₁₋₅ alkyl, and
   C₁₋₅ alkoxy;
or
Z₁ and Z₂ are bonded to each other to form a ring and -Z₁-Z₂- is —(CH₂)ₘ— or —(CH₂)ₙ—CH═CH—(CH₂)ₒ—; wherein one —CH₂— group of -Z₁-Z₂- can optionally be replaced by C(O), NR₅, O, S, S(O), or S(O)₂; wherein m is 2, 3, 4, 5, or 6; n and o are each independently 0, 1, 2, 3, or 4 provided that n+o=0, 1, 2, 3, or 4; and R₅ is hydrogen or C₁₋₅ alkyl;
Y is —S(O)₂—, —C(O)—, —C(O)NR₆—, —C(S)NR₆—, —C(O)O—, or —(CH₂)ₚ—; wherein R₆ is hydrogen or C₁₋₅ alkyl; p is 0, 1, 2, 3, 4, or 5; and
q is 0 or 1;
   wherein carbocyclic aryl is phenyl, naphthyl, or anthranyl;
   carbocyclyl is 1-oxo-indanyl, 9H-fluorenyl, 9-oxo-fluorenyl, anthraquinonyl, C-fluoren-9-ylidene, or indanyl;
   heterocyclyl is 1,2,3-thiadiazolyl, 1,2,3-triazolyl, 1,3-dioxo-isoindolyl, 1H-indolyl, 1H-pyrrolyl, 2,3-dihydro-benzo[1,4]dioxinyl, 2,3-dihydro-benzofuryl, 2H-benzopyranyl, 2-oxo-benzopyranyl, 2-oxo-pyrrolidinyl, 4-oxo-3,4-dihydro-phthalazinyl, 4-oxo-benzopyranyl, 9H-xanthenyl, benzimidazolyl, benzo[1,3]dioxolyl, benzo[2,1,3]oxadiazolyl, benzo[1,2,5]oxadiazolyl, benzo[b]thienyl, benzofuryl, benzothiazolyl, cinnolyl, furyl, imidazolyl, isoxazolyl, morpholino, morpholinyl, oxazolyl, piperazyl, piperidyl, pyrazolyl, pyrazinyl, pyridyl, pyrimidyl, pyrrolidinyl, quinolyl, quinoxalyl, thiazolidyl, thiazolyl, or thienyl; and
   halogen is fluoro, chloro, bromo, or iodo;
or a pharmaceutically acceptable salt, hydrate, or solvate thereof.

One aspect of the present invention pertains to pharmaceutical compositions comprising at least one compound, as described herein, in combination with a pharmaceutically acceptable carrier.

One aspect of the present invention pertains to methods for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from said condition a therapeutically effective amount of a compound, as described herein, or a pharmaceutical composition thereof.

One aspect of the present invention pertains to methods for the prophylaxis or treatment of an eating disorder, obesity or an obesity related disorder comprising administering to an individual suffering from the condition a therapeutically effective amount of a compound, as described herein, or a pharmaceutical composition thereof.

One aspect of the present invention pertains to methods for the prophylaxis or treatment of anxiety, depression, schizophrenia, addiction, or epilepsy comprising administering to an individual suffering from the condition a therapeutically effective amount of a compound, as described herein, or a pharmaceutical composition.

One aspect of the present invention pertains to compounds of the present invention, as described herein, or a pharmaceutical composition thereof, for use in a method of treatment of the human or animal body by therapy.

One aspect of the present invention pertains to compounds of the present invention, as described herein, or a pharmaceutical composition thereof, for use in a method of prophylaxis or treatment of an eating disorder, obesity or an obesity related disorder of the human or animal body by therapy.

One aspect of the present invention pertains to compounds of the present invention, as described herein, or a pharmaceutical composition thereof, for use in a method of prophylaxis or treatment of anxiety, depression, schizophrenia, addiction, or epilepsy of the human or animal body by therapy.

One aspect of the present invention pertains to compounds of the present invention, as described herein, for the manufacture of a medicament for use in the prophylaxis or treatment of an eating disorder, obesity or obesity related disorders.

One aspect of the present invention pertains to compounds of the present invention, as described herein, for the manufacture of a medicament for use in the prophylaxis or treatment of anxiety, depression, schizophrenia, addiction, or epilepsy.

One aspect of the present invention pertains to methods of decreasing food intake of an individual comprising administering to the individual a therapeutically effective amount of a compound, as described herein, or a pharmaceutical composition thereof.

One aspect of the present invention pertains to methods of inducing satiety in an individual comprising administering to said individual a therapeutically effective amount of a compound, as described herein, or a pharmaceutical composition thereof.

One aspect of the present invention pertains to methods of controlling or reducing weight gain in an individual comprising administering to said individual a therapeutically effective amount of a compound, as described herein, or a pharmaceutical composition thereof.

One aspect of the present invention pertains to methods of modulating a MCH receptor in an individual comprising contacting the receptor with a compound, as described herein. In some embodiments, the compound is an antagonist. In some embodiments, the modulation of the MCH receptor is for the prophylaxis or treatment of an eating disorder, obesity or obesity related disorder. In some embodiments, the modulation of the MCH receptor reduces food intake of the individual. In some embodiments, the modulation of the MCH receptor induces satiety in the individual. In some embodiments, the modulation of the MCH receptor controls or reduces weight gain of the individual. In some embodiments, the modulation of the MCH receptor is for prophylaxis or treatment of anxiety, depression, schizophrenia, addiction, or epilepsy.

In some embodiments, the individual is a mammal.

In some embodiments, the mammal is a human.

In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

One aspect of the present invention pertains to methods of producing a pharmaceutical composition comprising admixing a compound, as described herein, and a pharmaceutically acceptable carrier.

### DETAILED DESCRIPTION OF THE INVENTION

One aspect of the present invention relates to certain substituted pyridine compounds represented by Formula (I): 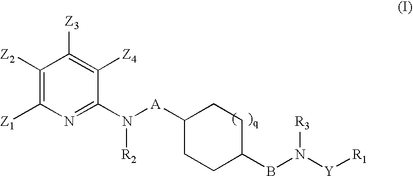
or a pharmaceutically acceptable salt, hydrate or solvate thereof, wherein R₁, R₂, R₃, A, B, Z₁, Z₂, Z₃, Z₄, Y, and q are as described herein, supra and infra.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

In some embodiments, compounds of the present invention are of Formula (I) wherein R₁ is selected from the group consisting of:
(i) C₁₋₁₀ alkyl substituted by substituent(s) independently selected from the group consisting of:
   oxo,
   C₁₋₅ alkoxy substituted by carbocyclic aryl,
   C₁₋₅ alkylcarbonyloxy,
   carbocyclic aryloxy,
   carbocyclic aryloxy substituted by substituent(s) independently selected from the group consisting of:
      halogen,
      nitro,
      C₁₋₅ alkyl, and
      C₁₋₅ alkyl substituted by oxo,
   heterocyclyloxy,
   heterocyclyloxy substituted by C₁₋₅ alkyl,
   C₁₋₅ alkoxycarbonyl,
   C₁₋₅ alkoxycarbonyl substituted by carbocyclic aryl,
   mono-carbocyclic arylamino,
   mono-carbocyclic arylamino substituted by hydroxy,
   di-carbocyclic arylamino,
   di-carbocyclic arylamino substituted by hydroxy,
   C₁₋₅ alkylcarbonylamino,
   heterocyclyl carbonylamino,
   carbocyclic arylsulfonylamino,
   carbocyclic arylsulfonylamino substituted by nitro,
   carbocyclic arylsulfonylamino substituted by C₁₋₅ alkyl,
   C₁₋₅ alkylthio,
   C₁₋₅ alkylthio substituted by carbocyclic aryl,
   carbocyclic arylthio,
   carbocyclic arylthio substituted by halogen,
   carbocyclic arylthio substituted by C₁₋₅ alkyl,
   carbocyclic arylsulfonyl,
   carbocyclic arylsulfonyl substituted by halogen,
   heterocyclylthio,
   heterocyclylthio substituted by C₁₋₅ alkyl,
   C₃₋₆ cycloalkyl,
   C₃₋₆ cycloalkenyl,
   carbocyclyl,
   carbocyclyl substituted by C₁₋₅ alkoxy,
   carbocyclic aryl,
   carbocyclic aryl substituted by substituent(s) independently selected from the group consisting of:
      halogen,
      hydroxy,
      nitro,
      C₁₋₅ alkyl,
      C₁₋₅ alkyl substituted by oxo,
      C₁₋₅ alkyl substituted by carbocyclic aryl,
      C₁₋₅ alkyl substituted by heterocyclyl,
      C₁₋₅ alkoxy,
      C₁₋₅ alkoxy substituted by halogen,
      C₁₋₅ alkoxy substituted by carbocyclic aryl,
      carbocyclic aryloxy,
      mono-carbocyclic arylamino,
      mono-carbocyclic arylamino substituted by halogen,
      di-carbocyclic arylamino,
      di-carbocyclic arylamino substituted by halogen,
      mono-carbocyclic arylaminocarbonyl,
      mono-carbocyclic arylaminocarbonyl substituted by substituent(s) selected from the group consisting of:
         halogen,
         C₁₋₅ alkyl,
         C₁₋₅ alkoxy, and
         C₁₋₅ alkoxy substituted by halogen,
      di-carbocyclic arylaminocarbonyl,
      di-carbocyclic arylaminocarbonyl substituted by substituent(s) selected from the group consisting of:
         halogen,
         C₁₋₅ alkyl,
         C₁₋₅ alkoxy, and
         C₁₋₅ alkoxy substituted by halogen,
      mercapto,
      C₁₋₅ alkylthio,
      C₁₋₅ alkylthio substituted by halogen,
      C₁₋₅ alkylsulfonyl,
      carbocyclic aryl, and
      heterocyclyl,
   heterocyclyl, and
   heterocyclyl substituted by substituent(s) independently selected from the group consisting of:
      C₁₋₅ alkyl,
      C₁₋₅ alkoxy,
      C₁₋₅ alkoxy substituted by carbocyclic aryl,
      carbocyclic aryl, and
      carbocyclic aryl substituted by halogen,
(ii) C₂₋₅ alkenyl substituted by substituent(s) independently selected from the group consisting of:
   oxo,
   carbocyclic aryl,
   carbocyclic aryl substituted by substituent(s) independently selected from the group consisting of:
      halogen,
      nitro,
      C₁₋₅ alkyl,
      C₁₋₅ alkyl substituted by halogen,
      C₁₋₅ alkoxy, and
      C₁₋₅ alkoxy substituted by halogen,
(iii) C₃₋₆ cycloalkyl substituted by substituent(s) independently selected from the group consisting of:
   C₁₋₅ alkyl,
   C₁₋₅ alkyl substituted by oxo,
   C₁₋₅ alkyl substituted by carbocyclic aryl,
   carbocyclic arylcarbonylamino, and
   carbocyclic aryl, and
   carbocyclic aryl by halogen,
(iv) carbocyclyl, and
   carbocyclyl substituted by nitro,
(v) carbocyclic aryl, and
   carbocyclic aryl substituted by substituent(s) independently selected from the group consisting of:
      halogen,
      hydroxy,
      cyano,
      nitro,
      C₁₋₉ alkyl,
      C₁₋₉ alkyl substituted by substituent(s) independently selected from the group consisting of:
         oxo,
         halogen,
         carbocyclic aryloxy,
         mono-carbocyclic arylaminocarbonyl,
         di-carbocyclic arylaminocarbonyl,
         mono-carbocyclic arylaminocarbonyl substituted by C₁₋₅ alkoxy,
         di-carbocyclic arylaminocarbonyl substituted by C₁₋₅ alkoxy,
         carbocyclic aryl,
         carbocyclic aryl substituted by substituent(s) independently selected from the group consisting of:
         halogen,
         C₁₋₅ alkyl, and
         C₁₋₅ alkyl substituted by halogen,
         heterocyclyl, and
         heterocyclyl substituted by C₁₋₅ alkyl,
      C₁₋₇ alkoxy,
      C₁₋₇ alkoxy substituted by halogen,
      C₁₋₇ alkoxy substituted by carbocyclic aryl,
      C₁₋₅ alkylcarbonyloxy,
      carbocyclic aryloxy,
      carbocyclic aryloxy substituted by C₁₋₅ alkoxy,
      C₁₋₅ alkoxycarbonyl,
      mono-C₁₋₅ alkylaminocarbonyl,
      di-C₁₋₅ alkylaminocarbonyl,
      mono-C₁₋₅ alkylaminocarbonyl substituted by carbocyclic aryl,
      di-C₁₋₅ alkylaminocarbonyl substituted by carbocyclic aryl,
      mono-carbocyclic arylaminocarbonyl,
      di-carbocyclic arylaminocarbonyl,
      mono-carbocyclic arylaminocarbonyl substituted by C₁₋₅ alkyl,
      di-carbocyclic arylaminocarbonyl substituted by C₁₋₅ alkyl,
      mono-C₁₋₅ alkylamino,
      di-C₁₋₅ alkylamino,
      carbocyclic arylsulfonylamino,
      carbocyclic arylsulfonylamino substituted by C₁₋₅ alkyl,
      C₁₋₅ alkylthio,
      C₁₋₅ alkylthio substituted by halogen,
      carbocyclic arylthio,
      carbocyclic arylthio substituted by cyano,
      C₁₋₅ alkylsulfonyl,
      mono-C₁₋₅ alkylaminosulfonyl,
      di-C₁₋₅ alkylaminosulfonyl,
      carbocyclic aryl,
      carbocyclic aryl substituted by substituent(s) independently selected from the group consisting of:
         C₁₋₇ alkyl, and
         C₁₋₇ alkyl substituted by halogen,
      heterocyclyl,
(vi) heterocyclyl, and
   heterocyclyl substituted by substituent(s) independently selected from the group consisting of:
      halogen,
      nitro,
      C₁₋₅ alkyl,
      C₁₋₅ alkyl substituted by substituent(s) independently selected from the group consisting of:
         halogen,
         oxo,
         carbocyclic aryl,
         carbocyclic aryl substituted by halogen, and
         heterocyclyl,
      C₁₋₅ alkoxy,
      carbocyclic aryloxy,
      carbocyclic aryloxy substituted by C₁₋₅ alkyl,
      C₁₋₅ alkylthio,
      carbocyclic arylthio,
      C₁₋₅ alkylsulfonyl,
      carbocyclic arylsulfonyl,
      carbocyclic arylsulfonyl substituted by halogen,
      carbocyclic arylsulfonyl substituted by C₁₋₅ alkyl,
      carbocyclic aryl,
      carbocyclic aryl substituted by substituent(s) independently selected from the group consisting of:
         halogen,
         nitro, and
         C₁₋₅ alkyl,
      heterocyclyl, and
      heterocyclyl substituted by substituent(s) independently selected from the group consisting of:
         C₁₋₅ alkyl, and
         C₁₋₅ alkyl substituted by halogen;
Z₁, Z₂, Z₃, and Z₄ are each independently hydrogen, halogen, cyano, nitro, carboxy, carbamoyl, C₁₋₅ alkyl, C₁₋₅ alkyl substituted by halogen, C₁₋₅ alkyl substituted by hydroxy, C₁₋₅ alkoxy, C₁₋₅ alkoxy substituted by halogen, C₁₋₅ alkoxy substituted by hydroxy, —CO₂R₄ₐ, —C(O)N(R₄ₐ)(R_{4b}), —N(R₄ₐ)(R_{4b}), or heterocyclyl; wherein R₄ₐ and R_{4b} are each independently hydrogen, C₁₋₅ alkyl, or C₁₋₅ alkyl substituted by substituent(s) independently selected from the group consisting of:
   halogen,
   hydroxy,
   carboxy,
   carbamoyl,
   C₁₋₅ alkoxy,
   amino,
   C₃₋₆ cycloalkyl,
   carbocyclic aryl,
   carbocyclic aryl substituted by substituent(s) independently selected from the group consisting of:
      halogen,
      C₁₋₅ alkyl,
      C₁₋₅ alkyl substituted by halogen,
      C₁₋₅ alkoxy,
      C₁₋₅ alkoxy substituted by halogen, and
      —SO₂NH₂,
   heterocyclyl, and
C₃₋₆ cycloalkyl, carbocyclic aryl, carbocyclic aryl substituted by substituent(s) independently selected from the group consisting of:
   halogen,
   C₁₋₅ alkyl, and
   C₁₋₅ alkoxy;
wherein Z₂ is not —C(O)N(R₄ₐ)(R_{4b});
or
Z₁ and Z₂ are bonded to each other to form a ring and -Z₁-Z₂- is —(CH₂)ₘ— or —(CH₂)ₙ —CH═CH—(CH₂)ₒ—; wherein one —CH₂— group of -Z₁-Z₂- can optionally be replaced by C(O), NR₅, O, S, S(O), or S(O)₂; wherein m is 2, 3, 4, 5, or 6; n and o are each independently 0, 1, 2, 3, or 4 provided that n+o=0, 1, 2, 3, or 4; and R₅ is hydrogen or C₁₋₅ alkyl;
Y is —S(O)₂—, —C(O)—, —C(S)NR₆—, —C(O)O—, or —(CH₂)ₚ—; wherein R₆ is hydrogen or C₁₋₅ alkyl; p is 0, 1, 2, 3, 4, or 5; and
q is 0 or 1;
   wherein carbocyclic aryl is phenyl, naphthyl, or anthranyl;
   carbocyclyl is 1-oxo-indanyl, 9H-fluorenyl, 9-oxo-fluorenyl, anthraquinonyl, C-fluoren-9-ylidene, or indanyl;
   heterocyclyl is 1,2,3-thiadiazolyl, 1,2,3-triazolyl, 1,3-dioxo-isoindolyl, 1H-indolyl, 1H-pyrrolyl, 2,3-dihydro-benzo[1,4]dioxinyl, 2,3-dihydro-benzofuryl, 2H-benzopyranyl, 2-oxo-benzopyranyl, 2-oxo-pyrrolidinyl, 4-oxo-3,4-dihydro-phthalazinyl, 4-oxo-benzopyranyl, 9H-xanthenyl, benzimidazolyl, benzo[1,3]dioxolyl, benzo[2,1,3]oxadiazolyl, benzo[1,2,5]oxadiazolyl, benzo[b]thienyl, benzofuryl, benzothiazolyl, cinnolyl, furyl, imidazolyl, isoxazolyl, morpholino, morpholinyl, oxazolyl, piperazyl, piperidyl, pyrazolyl, pyrazinyl, pyridyl, pyrimidyl, pyrrolidinyl, quinolyl, quinoxalyl, thiazolidyl, thiazolyl, or thienyl; and
   halogen is fluoro, chloro, bromo, or iodo;
or a pharmaceutically acceptable salt, hydrate, or solvate thereof.

In some embodiments, compounds of the present invention are of Formula (I) wherein Formula (I) is Formula (Ia) or a pharmaceutically acceptable salt, hydrate, or solvate thereof.

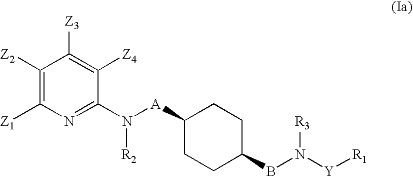

In some embodiments, compounds of the present invention are of Formula (Ia) wherein R₁ is elected from the group consisting of:
(i) C₁₋₁₀ alkyl substituted by substituent(s) independently selected from the group consisting of:
   oxo,
   C₁₋₅ alkoxy substituted by carbocyclic aryl,
   carbocyclic aryloxy,
   carbocyclic aryloxy substituted by substituent(s) independently selected from the group consisting of:
      halogen,
      nitro,
      C₁₋₅ alkyl, and
      C₁₋₅ alkyl substituted by oxo,
   C₁₋₅ alkoxycarbonyl,
   C₁₋₅ alkoxycarbonyl substituted by carbocyclic aryl,
   mono-carbocyclic arylamino,
   di-carbocyclic arylamino,
   heterocyclyl carbonylamino,
   carbocyclic arylsulfonylamino,
   carbocyclic arylsulfonylamino substituted by nitro,
   carbocyclic arylsulfonylamino substituted by C₁₋₅ alkyl,
   C₁₋₅ alkylthio,
   C₁₋₅ alkylthio substituted by carbocyclic aryl,
   carbocyclic arylthio,
   carbocyclic arylthio substituted by halogen,
   carbocyclic arylthio substituted by C₁₋₅ alkyl,
   carbocyclic arylsulfonyl,
   carbocyclic arylsulfonyl substituted by halogen,
   heterocyclylthio,
   heterocyclylthio substituted by C₁₋₅ alkyl,
   C₃₋₆ cycloalkyl,
   C₃₋₆ cycloalkenyl,
   carbocyclyl,
   carbocyclyl substituted by C₁₋₅ alkoxy,
   carbocyclic aryl,
   carbocyclic aryl substituted by substituent(s) independently selected from the group consisting of:
      halogen,
      nitro,
      C₁₋₅ alkyl,
      C₁₋₅ alkyl substituted by oxo,
      C₁₋₅ alkyl substituted by carbocyclic aryl,
      C₁₋₅ alkyl substituted by heterocyclyl,
      C₁₋₅ alkoxy,
      C₁₋₅ alkoxy substituted by halogen,
      C₁₋₅ alkoxy substituted by carbocyclic aryl,
      carbocyclic aryloxy,
      mono-carbocyclic arylaminocarbonyl,
      mono-carbocyclic arylaminocarbonyl substituted by substituent(s) selected from the group consisting of:
         halogen,
         C₁₋₅ alkyl,
         C₁₋₅ alkoxy, and
         C₁₋₅ alkoxy substituted by halogen,
      di-carbocyclic arylaminocarbonyl,
      di-carbocyclic arylaminocarbonyl substituted by substituent(s) selected from the group consisting of:
         halogen,
         C₁₋₅ alkyl,
         C₁₋₅ alkoxy, and
         C₁₋₅ alkoxy substituted by halogen,
      C₁₋₅ alkylsulfonyl,
      carbocyclic aryl, and
      heterocyclyl,
   heterocyclyl, and
   heterocyclyl substituted by substituent(s) independently selected from the group consisting of:
      C₁₋₅ alkyl,
      C₁₋₅ alkoxy,
      C₁₋₅ alkoxy substituted by carbocyclic aryl,
      carbocyclic aryl, and
      carbocyclic aryl substituted by halogen,
(ii) C₂₋₅ alkenyl substituted by substituent(s) independently selected from the group consisting of:
   oxo,
   carbocyclic aryl,
   carbocyclic aryl substituted by substituent(s) independently selected from the group consisting of:
      halogen,
      nitro,
      C₁₋₅ alkyl, and
      C₁₋₅ alkyl substituted by halogen,
(iii) C₃₋₆ cycloalkyl substituted by substituent(s) independently selected from the group consisting of:
   C₁₋₅ alkyl,
   C₁₋₅ alkyl substituted by oxo,
   C₁₋₅ alkyl substituted by carbocyclic aryl,
   carbocyclic arylcarbonylamino, and
   carbocyclic aryl, and
   carbocyclic aryl by halogen,
(iv) carbocyclyl, and
   carbocyclyl substituted by nitro,
(v) carbocyclic aryl, and
   carbocyclic aryl substituted by substituent(s) independently selected from the group consisting of:
      halogen,
      cyano,
      nitro,
      C₁₋₉ alkyl,
      C₁₋₉ alkyl substituted by substituent(s) independently selected from the group consisting of:
         oxo,
         halogen,
         carbocyclic aryloxy,
         mono-carbocyclic arylaminocarbonyl,
         di-carbocyclic arylaminocarbonyl,
         mono-carbocyclic arylaminocarbonyl substituted by C₁₋₅ alkoxy,
         di-carbocyclic arylaminocarbonyl substituted by C₁₋₅ alkoxy,
         carbocyclic aryl,
         carbocyclic aryl substituted by substituent(s) independently selected from the group consisting of:
         halogen,
         C₁₋₅ alkyl, and
         C₁₋₅ alkyl substituted by halogen,
         heterocyclyl, and
         heterocyclyl substituted by C₁₋₅ alkyl,
      C₁₋₇ alkoxy,
      C₁₋₇ alkoxy substituted by halogen,
      C₁₋₇ alkoxy substituted by carbocyclic aryl,
      carbocyclic aryloxy,
      carbocyclic aryloxy substituted by C₁₋₅ alkoxy,
      C₁₋₅ alkoxycarbonyl,
      mono-C₁₋₅ alkylaminocarbonyl,
      di-C₁₋₅ alkylaminocarbonyl,
      mono-C₁₋₅ alkylaminocarbonyl substituted by carbocyclic aryl,
      di-C₁₋₅ alkylaminocarbonyl substituted by carbocyclic aryl,
      mono-carbocyclic arylaminocarbonyl,
      di-carbocyclic arylaminocarbonyl,
      mono-carbocyclic arylaminocarbonyl substituted by C₁₋₅ alkyl,
      di-carbocyclic arylaminocarbonyl substituted by C₁₋₅ alkyl,
      mono-C₁₋₅ alkylamino,
      di-C₁₋₅ alkylamino,
      carbocyclic arylsulfonylamino,
      carbocyclic arylsulfonylamino substituted by C₁₋₅ alkyl,
      C₁₋₅ alkylthio,
      C₁₋₅ alkylthio substituted by halogen,
      carbocyclic arylthio,
      carbocyclic arylthio substituted by cyano,
      C₁₋₅ alkylsulfonyl,
      mono-C₁₋₅ alkylaminosulfonyl,
      di-C₁₋₅ alkylaminosulfonyl,
      carbocyclic aryl,
      carbocyclic aryl substituted by substituent(s) independently selected from the group consisting of:
         C₁₋₇ alkyl, and
         C₁₋₇ alkyl substituted by halogen,
      heterocyclyl,
(vi) heterocyclyl, and
   heterocyclyl substituted by substituent(s) independently selected from the group consisting of:
      halogen,
      nitro,
      C₁₋₅ alkyl,
      C₁₋₅ alkyl substituted by substituent(s) independently selected from the group consisting of:
         halogen,
         oxo, and
         heterocyclyl,
      C₁₋₅ alkoxy,
      carbocyclic aryloxy,
      C₁₋₅ alkylthio,
      C₁₋₅ alkylsulfonyl,
      carbocyclic arylsulfonyl,
      carbocyclic arylsulfonyl substituted by halogen,
      carbocyclic arylsulfonyl substituted by C₁₋₅ alkyl,
      carbocyclic aryl;
R₂ and R₃ are each hydrogen; and A and B are each independently a single bond or —CH₂—, provided that A is not —CH₂— when B is —CH₂—;
Z₁ and Z₂ are each independently hydrogen, halogen, C₁₋₅ alkyl, or —N(R₄ₐ)(R_{4b}); Z₃ is hydrogen, cyano, nitro, carbamoyl, C₁₋₅ alkyl, C₁₋₅ alkyl substituted by hydroxy, C₁₋₅ alkoxy, —C(O)N(R₄ₐ)(R_{4b}), —N(R₄ₐ)(R_{4b}), morpholinyl, pyrrolidinyl, or imidazolyl; wherein R₄ₐ and R_{4b} are each independently hydrogen, C₁₋₅ alkyl, or C₁₋₅ alkyl substituted by carbocyclic aryl; Z₄ is hydrogen, halogen, or C₁₋₅ alkyl;
or
Z₁ and Z₂ are bonded to each other to form a ring and -Z₁-Z₂- is —(CH₂)ₘ—; wherein m is 3 or 4; and
Y is —S(O)₂—, —C(O)—, —C(S)NH—, —C(O)O—, or —CH₂—;
   wherein carbocyclic aryl is phenyl, naphthyl, or anthranyl;
   carbocyclyl is 1-oxo-indanyl, 9H-fluorenyl, 9-oxo-fluorenyl, anthraquinonyl, C-fluoren-9-ylidene, or indanyl;
   heterocyclyl is 1H-indolyl, 1H-pyrrolyl, 2,3-dihydro-benzo[1,4]dioxinyl, 2H-benzopyranyl, 2-oxo-benzopyranyl, 2-oxo-pyrrolidinyl, 4-oxo-3,4-dihydro-phthalazinyl, 4-oxo-benzopyranyl, 9H-xanthenyl, benzo[1,3]dioxolyl, benzo[2,1,3]oxadiazolyl, benzo[1,2,5]oxadiazolyl, benzo[b]thienyl, cinnolyl, furyl, imidazolyl, morpholinyl, oxazolyl, pyrazolyl, pyridyl, pyrimidyl, pyrrolidinyl, quinoxalyl, thiazolyl, or thienyl; and
   halogen is fluoro, chloro, bromo, or iodo;
or a pharmaceutically acceptable salt, hydrate, or solvate thereof.

In some embodiments, compounds of the present invention are of Formula (Ia) wherein R₁ is elected from the group consisting of:
(i) C₁₋₅ alkyl substituted by substituent(s) independently selected from the group consisting of:
   carbocyclic aryloxy,
   carbocyclic aryloxy substituted by halogen, and
   carbocyclic aryl,
(ii) C₃₋₆ cycloalkyl substituted by substituent(s) independently selected from the group consisting of:
   carbocyclic aryloxy, and
   carbocyclic aryloxy substituted by halogen,
(iii) carbocyclic aryl, and
   carbocyclic aryl substituted by substituent(s) independently selected from the group consisting of:
      halogen,
      nitro,
      C₁₋₅ alkyl,
      C₁₋₅ alkyl substituted by halogen,
      C₁₋₅ alkoxy,
      C₁₋₅ alkoxy substituted by halogen,
      mono-C₁₋₅ alkylaminosulfonyl,
      di-C₁₋₅ alkylaminosulfonyl,
      carbocyclic aryloxy, and
      carbocyclic aryloxy substituted by C₁₋₅ alkoxy,
(iv) heterocyclyl,
   heterocyclyl substituted by halogen, and
   heterocyclyl substituted by carbocyclic aryloxy;
R₂ and R₃ are each hydrogen; A is a single bond; and B is a single bond or —CH₂—;
Z₁ and Z₂ are each independently hydrogen, C₁₋₅ alkyl, or —N(R₄ₐ)(R_{4b}); Z₃ is hydrogen, C₁₋₅ alkyl, C₁₋₅ alkoxy, —N(R₄ₐ)(R_{4b}), or pyrrolidinyl; wherein R₄ₐ and R_{4b} are each independently hydrogen, C₁₋₅ alkyl, or C₁₋₅ alkyl substituted by carbocyclic aryl; Z₄ is hydrogen or C₁₋₅ alkyl;
or
Z₁ and Z₂ are bonded to each other to form a ring and -Z₁-Z₂- is —(CH₂)ₘ—; wherein m is 3 or 4; and
Y is —C(O)—, —C(S)NH—, —C(O)O—, or —CH₂—;
   wherein carbocyclic aryl is phenyl or naphthyl;
   heterocyclyl is pyridyl, pyrrolidinyl, benzo[2,1,3]oxadiazolyl, or benzo[1,2,5]oxadiazolyl; and
   halogen is fluoro, chloro, or bromo;
or a pharmaceutically acceptable salt, hydrate, or solvate thereof.

In some embodiments, compounds of the present invention are of Formula (Ia) wherein R₁ is selected from the group consisting of:
(i) C₁₋₅ alkyl substituted by substituent(s) independently selected from the group consisting of:
   carbocyclic aryloxy, and
   carbocyclic aryloxy substituted by halogen,
(ii) C₃₋₆ cycloalkyl substituted by substituent(s) independently selected from the group consisting of:
   carbocyclic aryl, and
   carbocyclic aryl substituted by halogen,
(iii) carbocyclic aryl, and
   carbocyclic aryl substituted by substituent(s) independently selected from the group consisting of:
      halogen,
      nitro,
      C₁₋₅ alkyl,
      C₁₋₅ alkyl substituted by halogen,
      C₁₋₅ alkoxy,
      C₁₋₅ alkoxy substituted by halogen,
      carbocyclic aryloxy, and
      carbocyclic aryloxy substituted by C₁₋₅ alkoxy,
(iv) heterocyclyl, and
   heterocyclyl substituted by halogen;
R₂ and R₃ are each hydrogen; A is a single bond; and B is a single bond or —CH₂—;
Z₁ and Z₂ are each independently hydrogen, C₁₋₅ alkyl, or —N(R₄ₐ)(R_{4b}); Z₃ is hydrogen, C₁₋₅ alkyl, or —N(R₄ₐ)(R_{4b}), wherein R₄ₐ and R_{4b} are each independently hydrogen or C₁₋₅ alkyl; Z₄ is hydrogen;
or
Z₁ and Z₂ are bonded to each other to form a ring and -Z₁-Z₂- is —(CH₂)ₘ—; wherein m is 3 or 4; and
Y is —C(O)—;
   wherein carbocyclic aryl is phenyl;
   heterocyclyl is pyridyl, benzo[2,1,3]oxadiazolyl, or benzo[1,2,5]oxadiazolyl; and
   halogen is fluoro, chloro, or bromo;
or a pharmaceutically acceptable salt, hydrate, or solvate thereof.

In some embodiments, compounds of the present invention are of Formula (I) wherein the compound is selected from the group consisting of:
3-chloro-4-fluoro-N-{cis-4-[(4-methoxypyridin-2-yl)amino]cyclohexyl}benzamide;
N²-{cis-4-[(3-chloro-4-fluorobenzyl)amino]cyclohexyl}-N⁴,N⁴,5-trimethylpyridine-2,4-diamine;
N-{cis-4-[(4-amino-5-methylpyridin-2-yl)amino]cyclohexyl}-3,4,5-trifluorobenzamide;
4-bromophenyl (cis-4-{[4-(dimethylamino)-5-methylpyridin-2-yl]amino}cyclohexyl)-carbamate;
3-chloro-4-fluoro-N-{cis-4-[(4-methylpyridin-2-yl)amino]cyclohexyl}benzamide;
3-chloro-4-fluoro-N-[cis-4-({5-methyl-4-[methyl(2-phenylethyl)amino]pyridin-2-yl}amino)-cyclohexyl]benzamide;
3-chloro-4-fluoro-N-{cis-4-[(6-methylpyridin-2-yl)amino]cyclohexyl}benzamide;
N-{cis-4-[(4-amino-5-methylpyridin-2-yl)amino]cyclohexyl}-3-chloro-4-fluorobenzamide;
3-chloro-4-fluoro-N-{cis-4-[(5-methyl-4-pyrrolidin-1-ylpyridin-2-yl)amino]cyclohexyl}-benzamide;
3-chloro-4-fluoro-N-{cis-4-[(5-methylpyridin-2-yl)amino]cyclohexyl}benzamide;
N-(3-chloro-4-fluorophenyl)-N′-(cis-4-{[4-(dimethylamino)-5-methylpyridin-2-yl]amino}-cyclohexyl)thiourea;
N-(3-chloro-4-fluorophenyl)-N′-(cis-4-{[4-(dimethylamino)-5-methylpyridin-2-yl]amino}-cyclohexyl)urea;
3-chloro-4-fluoro-N-{cis-4-[(3,5,6-trimethylpyridin-2-yl)amino]cyclohexyl}benzamide;
3-chloro-N-(cis-4-{[4-(dimethylamino)-6-methylpyridin-2-yl]amino}cyclohexyl)-4-fluorobenzamide;
N-(cis-4-{[4-(dimethylamino)-6-methylpyridin-2-yl]amino}cyclohexyl)-3,4-difluorobenzamide;
N-(cis-4-{[4-(dimethylamino)-6-methylpyridin-2-yl]amino}cyclohexyl)-2-(4-methoxyphenoxy)-5-nitrobenzamide;
N-(cis-4-{[4-(dimethylamino)-6-methylpyridin-2-yl]amino}cyclohexyl)-2,1,3-benzoxadiazole-5-carboxamide;
1-(4-chlorophenyl)-N-(cis-4-{[4-(dimethylamino)-6-methylpyridin-2-yl]amino}cyclohexyl)cyclopentanecarboxamide;
N-(cis-4-{[4-(dimethylamino)-6-methylpyridin-2-yl]amino}cyclohexyl)-3-nitrobenzamide;
2-(4-chlorophenoxy)-N-(cis-4-{[4-(dimethylamino)-6-methylpyridin-2-yl]amino}cyclohexyl)acetamide; and
4-chloro-N-(cis-4-{[4-(dimethylamino)-6-methylpyridin-2-yl]amino}cyclohexyl)benzamide;
   or a pharmaceutically acceptable salt, hydrate, or solvate thereof.

In some embodiments, compounds of the present invention are of Formula (I) wherein the compound is selected from the group consisting of:
3-chloro-N-[(cis-4-{[4-(dimethylamino)-5-methylpyridin-2-yl]amino}cyclohexyl)methyl]-4-fluorobenzamide;
3-chloro-N-[cis-4-({4-[ethyl(methyl)amino]-5-methylpyridin-2-yl}amino)cyclohexyl]-4-fluorobenzamide;
3,4,5-trifluoro-N-(cis-4-{[5-methyl-4-(methylamino)pyridin-2-yl]amino}cyclohexyl)-benzamide;
N-(cis-4-{[4-(dimethylamino)-5-methylpyridin-2-yl]amino}cyclohexyl)-3-(trifluoromethoxy)benzamide;
5-bromo-N-(cis-4-{[4-(dimethylamino)-5-methylpyridin-2-yl]amino}cyclohexyl)-nicotinamide;
3-chloro-N-(cis-4-{[6-(dimethylamino)pyridin-2-yl]amino}cyclohexyl)-4-fluorobenzamide;
3-chloro-4-fluoro-N-(cis-4-{[5-methyl-4-(methylamino)pyridin-2-yl]amino}cyclohexyl)-benzamide;
N-(cis-4-{[4-(dimethylamino)-5-methylpyridin-2-yl]amino}cyclohexyl)-4-fluoro-3-(trifluoromethyl)benzamide;
N-(cis-4-{[4-(dimethylamino)-5-methylpyridin-2-yl]amino}cyclohexyl)-3-(trifluoromethyl)-benzamide;
3,5-dichloro-N-(cis-4-{[4-(dimethylamino)-5-methylpyridin-2-yl]amino}cyclohexyl)-benzamide;
3-chloro-N-(cis-4-{[4-(dimethylamino)-5-methylpyridin-2-yl]amino}cyclohexyl)benzamide;
3,4-dichloro-N-(cis-4-{[4-(dimethylamino)-5-methylpyridin-2-yl]amino}cyclohexyl)-benzamide;
3-chloro-N-(cis-4-{[4-(dimethylamino)-5-methylpyridin-2-yl]amino}cyclohexyl)-5-fluorobenzamide;
3,4,5-trifluoro-N-[cis-4-(5,6,7,8-tetrahydroquinolin-2-ylamino)cyclohexyl]benzamide;
3-chloro-4-fluoro-N-[cis-4-(5,6,7,8-tetrahydroquinolin-2-ylamino)cyclohexyl]benzamide;
N-(cis-4-{[4-(dimethylamino)pyridin-2-yl]amino}cyclohexyl)-3,4,5-trifluorobenzamide;
N-(cis-4-{[4-(dimethylamino)-5-methylpyridin-2-yl]amino}cyclohexyl)-3,4-difluorobenzamide;
3-chloro-N-(cis-4-{[4-(dimethylamino)pyridin-2-yl]amino}cyclohexyl)-4-fluorobenzamide;
3-chloro-N-{cis-4-[(5,6-dimethylpyridin-2-yl)amino]cyclohexyl}-4-fluorobenzamide;
N-(cis-4-{[4-(dimethylamino)-6-methylpyridin-2-yl]amino}cyclohexyl)-3,4,5-trifluorobenzamide;
N-(cis-4-{[4-(dimethylamino)-5-methylpyridin-2-yl]amino}cyclohexyl)-3,4,5-trifluorobenzamide;
3-chloro-N-(cis-4-{[4-(dimethylamino)-6-methylpyridin-2-yl]amino}cyclohexyl)-4-fluorobenzamide;
3-chloro-4-fluoro-N-{cis-4-[(4-methyl-5,6,7,8-tetrahydroquinolin-2-yl)amino]cyclohexyl}-benzamide;
3-chloro-4-fluoro-N-{cis-4-[(4-methyl-6,7-dihydro-5H-cyclopenta[b]pyridin-2-yl)amino]-cyclohexyl}benzamide;
3-chloro-N-(cis-4-{[4-(dimethylamino)-5-methylpyridin-2-yl]amino}cyclohexyl)-4-fluorobenzamide;
3-chloro-4-fluoro-N-{cis-4-[(4,5,6-trimethylpyridin-2-yl)amino]cyclohexyl}benzamide;
3-chloro-N-{cis-4-[(4,5-dimethylpyridin-2-yl)amino]cyclohexyl}-4-fluorobenzamide;
3-chloro-N-{cis-4-[(4,6-dimethylpyridin-2-yl)amino]cyclohexyl}-4-fluorobenzamide;
N-(cis-4-{[2-(4-chlorophenoxy)ethyl]amino}cyclohexyl)-N,N-6-trimethylpyridine-2,4-diamine;
N-(cis-4-{[4-(dimethylamino)-6-methylpyridin-2-yl]amino}cyclohexyl)-2-(1-naphthyl)acetamide;
N-(cis-4-{[4-(dimethylamino)-6-methylpyridin-2-yl]amino}cyclohexyl)-3-(trifluoromethyl)benzamide;
N-(cis-4-{[4-(dimethylamino)-6-methylpyridin-2-yl]amino}cyclohexyl)-4-[(dipropylamino)sulfonyl]benzamide; and
N-(cis-4-{[4-(dimethylamino)-6-methylpyridin-2-yl]amino}cyclohexyl)-2-phenoxynicotinamide;

or a pharmaceutically acceptable salt, hydrate, or solvate thereof.

In some embodiments, compounds of the present invention are of Formula (Ia) wherein R₁ is selected from hydrogen or —CO₂Bn (Bn is a benzyl group); R₂ and R₃ are each hydrogen; A and B are each independently a single bond or —CH₂—, provided that A is not —CH₂— when B is —CH₂—; Z₁ and Z₂ are each independently hydrogen, halogen, C₁₋₅ alkyl, or —N(R₄ₐ)(R_{4b}); Z₃ is hydrogen, cyano, nitro, carbamoyl, C₁₋₅ alkyl, C₁₋₅ alkyl substituted by hydroxy, C₁₋₅ alkoxy, —C(O)N(R₄ₐ)(R_{4b}), —N(R₄ₐ)(R_{4b}), morpholinyl, pyrrolidinyl, or imidazolyl; wherein R₄ₐ and R_{4b} are each independently hydrogen, C₁₋₅ alkyl, or C₁₋₅ alkyl substituted by carbocyclic aryl; Z₄ is hydrogen, halogen, or C₁₋₅ alkyl; or Z₁ and Z₂ are bonded to each other to form a ring and -Z₁-Z₂- is —(CH₂)ₘ—; wherein m is 3 or 4; Y is a single bond; and q is 0 or 1; or a pharmaceutically acceptable salt, hydrate, or solvate thereof.

In some embodiments, compounds of the present invention are of Formula (Ia) wherein R₁ is selected from hydrogen or —CO₂Bn (Bn is a benzyl group); R₂ and R₃ are each hydrogen; A is a single bond; B is a single bond or —CH₂—; Z₁ and Z₂ are each independently hydrogen, C₁₋₅ alkyl, or —N(R₄ₐ)(R_{4b}); Z₃ is hydrogen, C₁₋₅ alkyl, or —N(R₄ₐ)(R_{4b}); wherein R₄ₐ and R_{4b} are each independently hydrogen or C₁₋₅ alkyl; Z₄ is hydrogen; or Z₁ and Z₂ are bonded to each other to form a ring and -Z₁-Z₂- is —(CH₂)ₘ—; wherein m is 3 or 4; and Y is a single bond; or a pharmaceutically acceptable salt, hydrate, or solvate thereof.

One aspect of the present invention pertains to pharmaceutical compositions comprising at least one compound, as described herein, in combination with a pharmaceutically acceptable carrier.

One aspect of the present invention pertains to methods for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from the condition a therapeutically effective amount of a compound, as described herein, or a pharmaceutical composition thereof.

One aspect of the present invention pertains to methods for the prophylaxis or treatment of an eating disorder, obesity or an obesity related disorder comprising administering to an individual suffering from the condition a therapeutically effective amount of a compound, as described herein, or a pharmaceutical composition thereof.

One aspect of the present invention pertains to methods for the prophylaxis or treatment of anxiety, depression, schizophrenia, addiction, or epilepsy comprising administering to an individual suffering from the condition a therapeutically effective amount of a compound, as described herein, or a pharmaceutical composition.

One aspect of the present invention pertains to compounds of the present invention, as described herein, or a pharmaceutical composition thereof, for use in a method of treatment of the human or animal body by therapy.

One aspect of the present invention pertains to compounds of the present invention, as described herein, or a pharmaceutical composition thereof, for use in a method of prophylaxis or treatment of an eating disorder, obesity or an obesity related disorder of the human or animal body by therapy.

One aspect of the present invention pertains to compounds of the present invention, as described herein, or a pharmaceutical composition thereof, for use in a method of prophylaxis or treatment of anxiety, depression, schizophrenia, addiction, or epilepsy of the human or animal body by therapy.

One aspect of the present invention pertains to compounds of the present invention, as described herein, for the manufacture of a medicament for use in the prophylaxis or treatment of an eating disorder, obesity or obesity related disorders.

One aspect of the present invention pertains to compounds of the present invention, as described herein, for the manufacture of a medicament for use in the prophylaxis or treatment of anxiety, depression, schizophrenia, addiction, or epilepsy.

One aspect of the present invention pertains to methods of decreasing food intake of an individual comprising administering to the individual a therapeutically effective amount of a compound, as described herein, or a pharmaceutical composition thereof.

One aspect of the present invention pertains to methods of inducing satiety in an individual comprising administering to said individual a therapeutically effective amount of a compound, as described herein, or a pharmaceutical composition thereof.

One aspect of the present invention pertains to methods of controlling or reducing weight gain in an individual comprising administering to said individual a therapeutically effective amount of a compound, as described herein, or a pharmaceutical composition thereof.

One aspect of the present invention pertains to methods of modulating a MCH receptor in an individual comprising contacting the receptor with a compound, as described herein. In some embodiments, the compound is an antagonist. In some embodiments, the modulation of the MCH receptor is for the prophylaxis or treatment of an eating disorder, obesity or obesity related disorder. In some embodiments, the modulation of the MCH receptor reduces food intake of the individual. In some embodiments, the modulation of the MCH receptor induces satiety in the individual. In some embodiments, the modulation of the MCH receptor controls or reduces weight gain of the individual. In some embodiments, the modulation of the MCH receptor is for prophylaxis or treatment of anxiety, depression, schizophrenia, addiction, or epilepsy.

In some embodiments, the individual is a mammal.

In some embodiments, the mammal is a human.

In some embodiments, the human has a body mass index of about 18.5 to about 45. In some embodiments, the human has a body mass index of about 25 to about 45. In some embodiments, the human has a body mass index of about 30 to about 45. In some embodiments, the human has a body mass index of about 35 to about 45.

One aspect of the present invention pertains to methods of producing a pharmaceutical composition comprising admixing a compound, as described herein, and a pharmaceutically acceptable carrier.

One embodiment of the invention includes any compound of the invention which selectively binds an MCH receptor, such selective binding is preferably demonstrated by a Ki for one or more other GPCR(s), preferably NPY, being at least 10-fold greater than the Ki for any particular MCH receptor, preferable MCHR1.

As used herein, the term “alkyl” is intended to denote hydrocarbon compounds including straight chain and branched chain, including for example but not limited to methyl, ethyl, n-propyl, isopropyl, n-butyl, sec-butyl, tert-butyl, n-pentyl, isopentyl, tert-pentyl, n-hexyl, and the like.

The term “alkoxy” is intended to denote substituents of the formula
—O-alkyl.

At various places in the present specification substituents of compounds of the invention are disclosed in groups. It is specifically intended that the invention include each and every individual subcombination of the members of such groups.

G-protein coupled receptors (GPCRs) represent a major class of cell surface receptors with which many neurotransmitters interact to mediate their effects. GPCRs are predicted to have seven membrane-spanning domains and are coupled to their effectors via G-proteins linking receptor activation with intracellular biochemical sequelae such as stimulation of adenylyl cyclase. Melanin Concentrating Hormone (MCH), a cyclic peptide, has been identified as the endogenous ligand of the orphan G-protein coupled receptor SLC-1. See, for example, Shimomura et al., Biochem. Biophys. Res. Commun. 261, 622-26 (1999). Studies have indicated that MCH acts as a neurotransmitter/modulator/regulator to alter a number of behavioral responses.

Mammalian MCH (19 amino acids) is highly conserved between rat, mouse, and human, exhibiting 100% amino acid identity, but its physiological roles are less clear. MCH has been reported to participate in a variety of processes including feeding, water balance, energy metabolism, general arousal/attention state, memory and cognitive functions, and psychiatric disorders. For reviews, see 1. Baker, Int. Rev. Cytol. 126:1-47 (1991); 2. Baker, TEM 5:120-126 (1994); 3. Nahon, Critical Rev. in Neurobiol 221:221-262, (1994); 4. Knigge et al., Peptides 18(7):1095-1097, (1996). The role of MCH in feeding or body weight regulation is supported by Qu et al., Nature 380:243-247, (1996), demonstrating that MCH is over expressed in the hypothalamus of ob/ob mice compared with ob/+mice, and that fasting further increased MCH mRNA in both obese and normal mice during fasting. MCH also stimulated feeding in normal rats when injected into the lateral ventricles as reported by Rossi et al., Endocrinology 138:351-355, (1997). MCH also has been reported to functionally antagonize the behavioral effects of α-MSH; see: Miller et al., Peptides 14:1-10, (1993); Gonzalez et al, Peptides 17:171-177, (1996); and Sanchez et al., Peptides 18:3933-396, (1997). In addition, stress has been shown to increase POMC mRNA levels while decreasing the MCH precursor preproMCH (ppMCH) mRNA levels; Presse et al., Endocrinology 131:1241-1250, (1992). Thus MCH can serve as an integrative neuropeptide involved in the reaction to stress, as well as in the regulation of feeding and sexual activity; Baker, Int. Rev. Cytol. 126:1-47, (1991); Knigge et al., Peptides 17:1063-1073, (1996).

The localization and biological activities of MCH peptide suggest that the modulation of MCH receptor activity can be useful in a number of therapeutic applications. MCH is expressed in the lateral hypothalamus, a brain area implicated in the regulation of thirst and hunger: Grillon et al., Neuropeptides 31:131-136, (1997); recently orexins A and B, which are potent orexigenic agents, have been shown to have very similar localization to MCH in the lateral hypothalamus; Sakurai et al., Cell 92:573-585 (1998). MCH mRNA levels in this brain region are increased in rats after 24 hours of food-deprivation; Herve and Fellmann, Neurpeptides 31:237-242 (1997); after insulin injection, a significant increase in the abundance and staining intensity of MCH immunoreactive perikarya and fibres was observed concurrent with a significant increase in the level of MCH mRNA; Bahjaoui-Bouhaddi et al., Neuropeptides 24:251-258, (1994). Consistent with the ability of MCH to stimulate feeding in rats; Rossi et al., Endocrinology 138:351-355, (1997); is the observation that MCH mRNA levels are upregulated in the hypothalami of obese ob/ob mice; Qu et al., Nature 380:243-247, (1996); and decreased in the hypothalami of rats treated with leptin, whose food intake and body weight gains are also decreased; Sahu, Endocrinology 139:795-798, (1998). MCH appears to act as a functional antagonist of the melanocortin system in its effects on food intake and on hormone secretion within the IPA (hypothalamopituitary/adrenal axis); Ludwig et al., Am. J. Physiol. Endocrinol. Metab. 274:E627-E633, (1998). Together these data suggest a role for endogenous MCH in the regulation of energy balance and response to stress, and provide a rationale for the development of specific compounds acting at MCH receptors for use in the treatment of obesity and stress-related disorders.

Accordingly, a MCH receptor antagonist is desirable for the prophylaxis or treatment of obesity or obesity related disorders. An obesity related disorder is a disorder that has been directly or indirectly associated to obesity, such as, type II diabetes, syndrome X, impaired glucose tolerance, dyslipidemia, hypertension, coronary heart disease and other cardiovascular disorders including atherosclerosis, insulin resistance associated with obesity and psoriasis, for treating diabetic complications and other diseases such as polycystic ovarian syndrome (PCOS), certain renal diseases including diabetic nephropathy, glomerulonephritis, glomerular sclerosis, nephrotic syndrome, hypertensive nephrosclerosis, end-stage renal diseases and microalbuminuria as well as certain eating disorders.

In species studied to date, a major portion of the neurons of the MCH cell group occupies a rather constant location in those areas of the lateral hypothalamus and subthalamus where they lie and may be a part of some of the so-called “extrapyramidal” motor circuits. These involve substantial striato- and pallidoftigal pathways involving the thalamus and cerebral cortex, hypothalamic areas, and reciprocal connections to subthalamic nucleus, substantia nigra, and mid-brain centers; Bittencourt et al., J. Comp. Neurol. 319:218-245, (1992). In their location, the MCH cell group may offer a bridge or mechanism for expressing hypothalamic visceral activity with appropriate and coordinated motor activity. Clinically it can be of some value to consider the involvement of this MCH system in movement disorders, such as Parkinson's disease and Huntingdon's Chorea in which extrapyramidal circuits are known to be involved.

Human genetic linkage studies have located authentic hMCH loci on chromosome 12 (12q23-24) and the variant hMCH loci on chromosome 5 (5q12-13) (Pedeutour et al., 1994). Locus 12q23-24 coincides with a locus to which autosomal dominant cerebellar ataxia type II (SCA2) has been mapped; Auburger et al., Cytogenet. Cell. Genet. 61:252-256, (1992); Twells et al., Cytogenet. Cell. Genet. 61:262-265, (1992). This disease comprises neurodegenerative disorders, including an olivopontocerebellar atrophy. Furthermore, the gene for Darier's disease, has been mapped to locus 12q23-24; Craddock et al., Hum. Mol. Genet. 2:1941-1943, (1993). Darier's disease is characterized by abnormalities I keratinocyte adhesion and mental illnesses in some families. In view of the functional and neuroanatomical patterns of the MCH neural system in the rat and human brains, the MCH gene can represent a good candidate for SCA2 or Darier's disease. Interestingly, diseases with high social impact have been mapped to this locus. Indeed, the gene responsible for chronic or acute forms of spinal muscular atrophies has been assigned to chromosome 5q12-13 using genetic linkage analysis; Melki et al., Nature (London) 344:767-768, (1990); Westbrook et al., Cytogenet. Cell. Genet. 61:225-231, (1992). Furthermore, independent lines of evidence support the assignment of a major schizophrenia locus to chromosome 5q11.2-13.3; Sherrington et al., Nature (London) 336:164-167, (1988); Bassett et al., Lancet 1:799-801, (1988); Gilliam et al., Genomics 5:940-944, (1989). The above studies suggest that MCH can play a role in neurodegenerative diseases and disorders of emotion.

Additional therapeutic applications for MCH-related compounds are suggested by the observed effects of MCH in other biological systems. For example, MCH can regulate reproductive functions in male and female rats. MCH transcripts and MCH peptide were found within germ cells in testes of adult rats, suggesting that MCH can participate in stem cell renewal and/or differentiation of early spermatocytes; Hervieu et al., Biology of Reduction 54:1161-1172, (1996). MCH injected directly into the medial preoptic area (MPOA) or ventromedial nucleus (VMN) stimulated sexual activity in female rats; Gonzalez et al., Peptides 17:171-177, (1996). In ovariectomized rats primed with estradiol, MCH stimulated luteinizing hormone (LH) release while anti-MCH antiserum inhibited LH release; Gonzalez et al., Neuroendocrinology 66:254-262, (1997). The zona incerta, which contains a large population of MCH cell bodies, has previously been identified as a regulatory site for the pre-ovulatory LH surge; MacKenzie et al., Neuroendocrinology 39:289-295, (1984). MCH has been reported to influence release of pituitary hormones including ACTH and oxytocin. MCH analogues can also be useful in treating epilepsy. In the PTZ seizure model, injection of MCH prior to seizure induction prevented seizure activity in both rats and guinea pigs, suggesting that MCH-containing neurons can participate in the neural circuitry underlying PTZ-induced seizure; Knigge and Wagner, Peptides 18:1095-1097, (1997). MCH has also been observed to affect behavioral correlates of cognitive functions. MCH treatment hastened extinction of the passive avoidance response in rats; McBride et al., Peptides 15:757-759, (1994); raising the possibility that MCH receptor antagonists can be beneficial for memory storage and/or retention. A possible role for MCH in the modulation or perception of pain is supported by the dense innervation of the periaqueductal grey (PAG) by MCH-positive fibers. Finally, MCH can participate in the regulation of fluid intake. ICV infusion of MCH in conscious sheep produced diuretic, natriuretic, and kaliuretic changes in response to increased plasma volume; Parkes, J. Neuroendocrinol. 8:57-63, (1996). Together with anatomical data reporting the presence of MCH in fluid regulatory areas of the brain, the results indicate that MCH can be an important peptide involved in the central control of fluid homeostasis in mammals.

In a recent citation MCHR1 antagonists surprisingly demonstrated their use as an anti-depressants and/or anti-anxiety agents. MCHR1 antagonists have been reported to show antidepressant and anxiolytic activities in rodent models, such as, social interaction, forced swimming test and ultrasonic vocalization. Therefore, MCHR1 antagonists could be useful to independently treat subjects with depression and/or anxiety. Also, MCHR1 antagonists could be useful to treat subjects that suffer from depression and/or anxiety and obesity.

This invention provides a method of treating an abnormality in a subject wherein the abnormality is alleviated by decreasing the activity of a mammalian MCH1 receptor which comprises administering to the subject an amount of a compound which is a mammalian MCH1 receptor antagonist effective to treat the abnormality. In separate embodiments, the abnormality is a regulation of a steroid or pituitary hormone disorder, an epinephrine release disorder, an anxiety disorder, genta gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a neuroendocrine disorder, a cognitive disorder, a memory disorder, a sensory modulation and transmission disorder, a motor coordination disorder, a sensory integration disorder, a motor integration disorder, a dopaininergic function disorder, a sensory transmission disorder, an olfaction disorder, a sympathetic innervation disorder, an affective disorder, a stress-related disorder, a fluid-balance disorder, a seizure disorder, pain, psychotic behavior, morphine tolerance, opiate addiction or migraine.

Compositions of the invention can conveniently be administered in unit dosage form and can be prepared by any of the methods well known in the pharmaceutical art, for example, as described in Remington's Pharmaceutical Sciences (Mack Pub. Co., Easton, Pa., 1980).

The compounds of the invention can be employed as the sole active agent in a pharmaceutical or can be used in combination with other active ingredients which could facilitate the therapeutic effect of the compound.

Compounds of the present invention or a solvate or physiologically functional derivative thereof can be used as active ingredients in pharmaceutical compositions, specifically as MCH receptor antagonists. By the term “active ingredient” is defined in the context of a “pharmaceutical composition” and shall mean a component of a pharmaceutical composition that provides the primary pharmaceutical benefit, as opposed to an “inactive ingredient” which would generally be recognized as providing no pharmaceutical benefit. The term “pharmaceutical composition” shall mean a composition comprising at one active ingredient and at least one ingredient that is not an active ingredient (for example and not limitation, a filler, dye, or a mechanism for slow release), whereby the composition is amenable to use for a specified, efficacious outcome in a mammal (for example, and not limitation, a human).

Pharmaceutical compositions, including, but not limited to, pharmaceutical compositions, comprising at least one compound of the present invention and/or an acceptable salt or solvate thereof (e.g., a pharmaceutically acceptable salt or solvate) as an active ingredient combined with at least one carrier or excipient (e.g., pharmaceutical carrier or excipient) can be used in the treatment of clinical conditions for which a MCH receptor antagonist is indicated. At least one compound of the present invention can be combined with the carrier in either solid or liquid form in a unit dose formulation. The pharmaceutical carrier must be compatible with the other ingredients in the composition and must be tolerated by the individual recipient. Other physiologically active ingredients can be incorporated into the pharmaceutical composition of the invention if desired, and if such ingredients are compatible with the other ingredients in the composition. Formulations can be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants can be used in tablets and capsules for oral administration. Liquid preparations for oral administration can be in the form of solutions, emulsions, aqueous or oily suspensions, and syrups. Alternatively, the oral preparations can be in the form of dry powder that can be reconstituted with water or another suitable liquid vehicle before use. Additional additives such as suspending or emulsifying agents, non-aqueous vehicles (including edible oils), preservatives, and flavorings and colorants can be added to the liquid preparations. Parenteral dosage forms can be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

It is noted that when the MCH receptor antagonists are utilized as active ingredients in a pharmaceutical composition, these are not intended for use only in humans, but in other non-human mammals as well. Indeed, recent advances in the area of animal health-care mandate that consideration be given for the use of MCH receptor antagonists for the treatment of obesity in domestic animals (e.g., cats and dogs), and MCH receptor antagonists in other domestic animals where no disease or disorder is evident (e.g., food-oriented animals such as cows, chickens, fish, etc.). Those of ordinary skill in the art are readily credited with understanding the utility of such compounds in such settings.

Pharmaceutically acceptable salts of the compounds of the invention can be prepared by reacting the free acid or base forms of these compounds with the appropriate base or acid in water, in an organic solvent, or in a mixture of the two; generally, nonaqueous media like ether, ethyl acetate, ethanol, isopropanol, dioxane, or acetonitrile are preferred. For instance, when the compound (I) possesses an acidic functional group, it can form an inorganic salt such as an alkali metal salt (e.g., sodium salt, potassium salt, etc.), an alkaline earth metal salt (e.g. calcium salt, magnesium salt, barium salt, etc.), and an ammonium salt. When the compound (I) possesses a basic functional group, it can form an inorganic salt (e.g., hydrochloride, sulfate, phosphate, hydrobromate, etc.) or an organic salt (e.g., acetate, maleate, fumarate, succinate, methanesulfonate, p-toluenesulfonate, citrate, tartrate, etc.).

When a compound of the invention contains optical isomers, stereoisomers, regio isomers, rotational isomers, a single substance and a mixture of them are included as a compound of the invention. For example, when a chemical formula is represented as showing no stereochemical designation(s), such as Formula (I), then all possible stereoisomer, optical isomers and mixtures thereof are considered within the scope of that formula. Accordingly, Formula (Ia), specifically designates the cis relationship between the two amino groups on the cyclohexyl ring and therefore this formula is also fully embraced by Formula (I).

### Preparation of Compound of Formula (I)

### General Synthetic Methods

The novel substituted pyridines of the present invention can be readily prepared according to a variety of synthetic manipulations, all of which would be familiar to one skilled in the art. Preferred methods for the preparation of compounds of the present invention include, but are not limited to, those described in Scheme 1-6.

The pyridine N-oxide (C) can be prepared as shown in Scheme 1. 2-Halopyridine (A) which is commercially available or synthesized by a well known method, wherein Z₁, Z₂, and Z₄ are as defined above and X is halogen such as fluoro, chloro, bromo, or iodo, is oxidized to 2-halopyridine N-oxide by an oxidizing reagent in an inert solvent. The oxidizing reagent includes hydrogen peroxide, a peracid (preferably peracetic acid or 3-chloroperoxybenzoic acid, etc.), an alkali metal peroxide, or an alkali peroxide. The inert solvent includes lower alkyl alcohol solvents (preferably methanol, ethanol, 2-propanol, or butanol, etc.), lower halocarbon solvents (preferably dichloromethane, dichloroethane, or chloroform, etc.), or aromatic solvents (preferably benzene or toluene, etc.). Reaction temperature ranges from about −50° C. to 150° C., preferably about −10° C. to 120° C. Nitration of 2-halopyridine N-oxide is achieved by fuming nitric acid with an acid (preferably sulfuric acid or acetic acid, etc.) to give 2-halo-4-nitro-pyridine N-oxide (B). Reaction temperature ranges from about −50° C. to 150° C., preferably about −10° C. to 120° C. Substitution of the nitro group with halogen is accomplished to provide 2,4-dihalopyridine N-oxide (C). The halogenating reagent includes halogen (preferably chlorine, bromine, or iodine, etc.), hydrohalic acid (preferably hydrochloric acid or hydrobromic acid, etc.), acetyl halide (preferably acetyl chloride, etc.), or lithium halide (preferably lithium chloride, etc.). The inert solvent includes amide solvents (preferably N,N-dimethylformamide or 1-methyl-pyrrolidin-2-one, etc.), lower halocarbon solvents (preferably dichloromethane, dichloroethane, chloroform, or carbon tetrachloride, etc.), aromatic solvents (preferably benzene or toluene, etc.), or lower alkylsulfoxide solvents (preferably dimethyl sulfoxide, etc.). Reaction temperature ranges from about −50° C. to 200° C., preferably about −10° C. to 180° C. Also reduction of the nitro group followed by Sandmeyer reaction can give 2,4-dihalopyridine N-oxide (C).

Alternatively, 2,4-dihalopyridine N-oxide (C) can be prepared from 2,4-dihydroxypyridine (D) which is commercially available or synthesized by a well known method. 2,4-Dihydroxypyridine (D) is converted to 2,4-dihalopyridine (E) by a halogenating reagent with or without a base. The halogenating reagent includes phosphorous oxychloride (POCl₃), phosphorous oxybromide (POBr₃), or phosphorus pentachloride (PCl₅). The base includes a tertiary amine (preferably N,N-diisopropylethylamine, etc.) or an aromatic amine (preferably N,N-dimethylaniline, etc.). Reaction temperature ranges from about 100° C. to 200° C., preferably about 140° C. to 180° C. N-Oxidation of 2,4-dihalopyridine (E) is achieved by the same manner as the oxidative method of 2-halopyridine (A) to provide 2,4-dihalopyridine N-oxide (C).

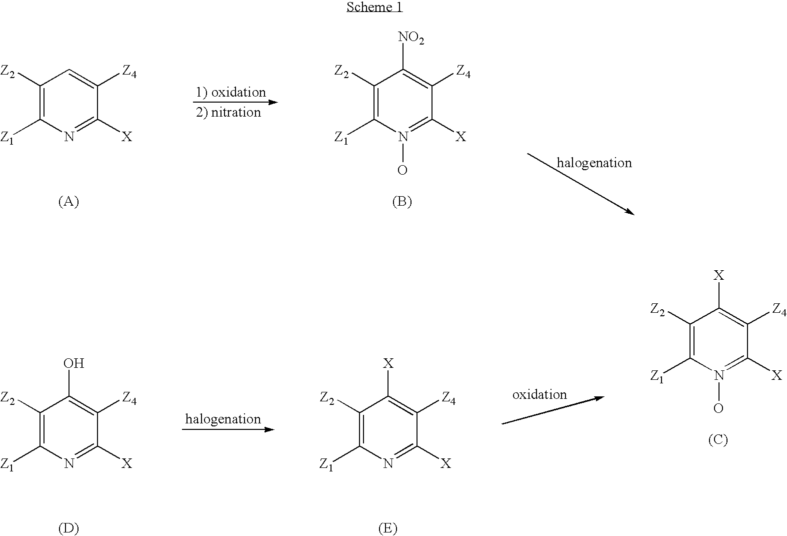

The common intermediate (I) of the novel substituted pyridines can be prepared as shown in Scheme 2. 2,4-Dihalopyridine N-oxide (C) is selectively substituted by the mono-protected diamine (F), wherein R₂, R₃, A, B, and q are as defined above and P is a protective group, with or without a base in an inert solvent to provide the coupling adduct (G). The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 150° C. Also this reaction can be carried out under microwave conditions. Subsequent amination at the 4-position is achieved by the same manner as the previous step with an appropriate amine. Simultaneously reduction of pyridine N-oxide to pyridine may happen in this condition.

Representative protecting groups suitable for a wide variety of synthetic transformations are disclosed in Greene and Wuts, Protective Groups in Organic Synthesis, second edition, John Wiley & Sons, New York, 1991, the disclosure of which is incorporated herein by reference in its entirety. The deprotection of the protective group leads to the common intermediate (I) of the novel substituted pyridines. Also reduction of pyridine N-oxide to pyridine may happen simultaneously in this condition. The pyridine N-oxide is reduced to the corresponding pyridine under transfer hydrogenation conditions using a palladium catalyst (preferably palladium metal on activated carbon, etc.) in an inert solvent, for instance lower alkyl alcohol solvents (preferably methanol, ethanol, or 2-propanol, etc.). Cyclohexene, 1,4-cyclohexadiene, formic acid, or salts of formic acid, such as potassium formate or ammonium formate, are commonly used as the hydrogen transfer reagent. Reaction temperature ranges from about 10° C. to 200° C., preferably about 50° C. to 150° C. Also the reduction can be carried out by a metal (preferably zinc, iron, or tin, etc.) in the presence of an acid (preferably hydrochloric acid or acetic acid, etc.). Reaction temperature ranges from about 10° C. to 200° C., preferably about 50° C. to 150° C. Furthermore the reduction can be performed by a usual hydrogenolysis method.

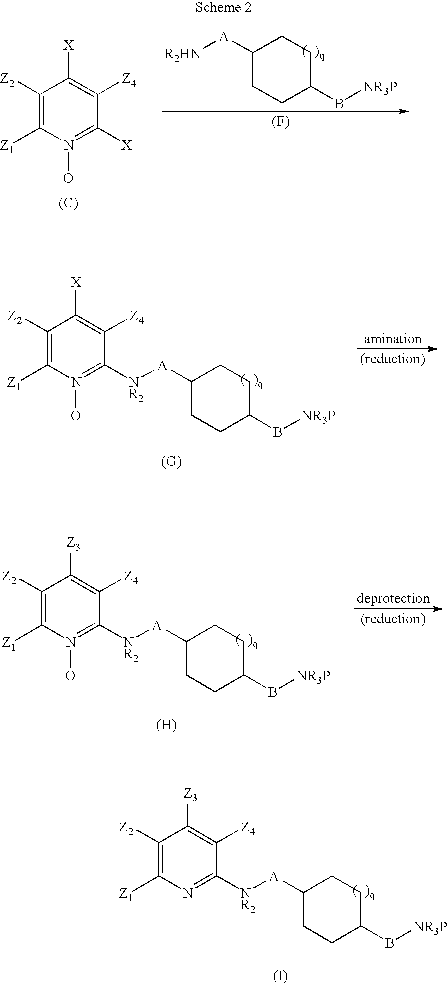

Alternatively the pyridine N-oxide derivatives (H) can be prepared from 2,6-dihalopyridine (J) which is commercially available or synthesized by a well known method as shown in Scheme 3. N-Oxidation of from 2,6-dihalopyridine (J) is achieved by the same manner as the oxidative method of 2-halopyridine (A) to provide 2,6-dihalopyridine N-oxide (K). Mono-substitution with the mono-protected diamine (F) is accomplished by the same manner as the method to get compound (G). Subsequent amination at the 6-position is achieved by the same manner as the previous step with an appropriate amine to give the pyridine N-oxide derivatives (H). Simultaneously reduction of pyridine N-oxide to pyridine may happen in this condition.

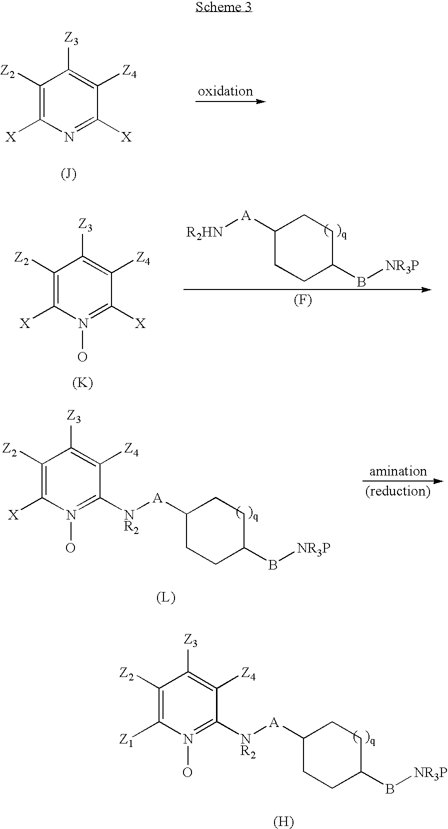

The conversion of the common intermediate (I) to the novel substituted pyridines (M) to (Q) of the present invention is outlined in Scheme 4.

The amine (I) is reacted with a carboxylic acid (R₁CO₂H) and a dehydrating condensing agent in an inert solvent with or without a base to provide the novel amide (M) of the present invention. The dehydrating condensing agent includes dicyclohexylcarbodiimide (DCC), 1-ethyl-3-(3-dimethylaminopropyl)carbodiimide hydrochloride (EDC.HCl), bromo-tris-pyrrolidino-phosphonium hexafluorophosphate (PyBroP), O-(7-azabenzotriazol-1-yl)-1,1,3,3-tetramethyluronium hexafluorophosphate (HATU), or 1-cyclohexyl-3-methylpolystyrene-carbodiimide. The base includes a tertiary amine (preferably N,N-diisopropylethylamine or triethylamine, etc.). The inert solvent includes lower halocarbon solvents (preferably dichloromethane, dichloroethane, or chloroform, etc.), ethereal solvents (preferably tetrahydrofuran or dioxane), nitrile solvents (preferably acetonitrile, etc.), or amide solvents (preferably N,N-dimethylformamide, etc.). In case of need, 1-hydroxybenzotriazole (HOBT), HOBT-6-carboxaamidomethyl polystyrene, or 1-hydroxy-7-azabenzotriazole (HOAT) can be used as a reactant agent. Reaction temperature ranges from about −20° C. to 50° C., preferably about 0° C. to 40° C.

Alternatively, the novel amide (M) of the present invention can be obtained by amidation reaction using an acid halide (R₁COX), wherein X is halogen such as chloro, bromo, or iodo, and a base in an inert solvent. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), amide solvents (preferably N,N-dimethylformamide, etc.), or aromatic solvents (preferably toluene or pyridine, etc.). Reaction temperature ranges from about −20° C. to 50° C., preferably about 0° C. to 40° C.

The novel amide (M) of the present invention is reacted with a reducing agent in an inert solvent to provide the novel amine (N) of the present invention. The reducing agent includes alkali metal aluminum hydrides (preferably lithium aluminum hydride), alkali metal borohydrides (preferably lithium borohydride), alkali metal trialkoxyaluminum hydrides (preferably lithium tri-tert-butoxyaluminum hydride), dialkylaluminum hydrides (preferably di-isobutylaluminum hydride), borane, dialkylboranes (preferably di-isoamyl borane), and alkali metal trialkylboron hydrides (preferably lithium triethylboron hydride). The inert solvent includes ethereal solvents (preferably tetrahydrofuran or dioxane) or aromatic solvents (preferably toluene, etc.). Reaction temperature ranges from about −78° C. to 200° C., preferably about 50° C. to 120° C.

Alternatively, the novel amine (N) of the present invention can be obtained by reductive amination reaction using aldehyde (R₁CHO) and a reducing agent in an inert solvent with or without an acid. The reducing agent includes sodium triacetoxyborohydride, sodium cyanoborohydride, sodium borohydride, or boran-pyridine complex, preferably sodium triacetoxyborohydride or sodium cyanoborohydride. The inert solvent includes lower alkyl alcohol solvents (preferably methanol or ethanol, etc.), lower halocarbon solvents (preferably dichloromethane, dichloroethane, or chloroform, etc.), ethereal solvents (preferably tetrahydrofuran or dioxane), or aromatic solvents (preferably toluene, etc.). The acid includes an inorganic acid (preferably hydrochloric acid or sulfuric acid) or an organic acid (preferably acetic acid). Reaction temperature ranges from about −20° C. to 120° C., preferably about 0° C. to 100° C. Also this reaction can be carried out under microwave conditions.

The amine (I) is reacted with a sulfonyl halide (R₁SO₂X), wherein X is halogen such as chloro, bromo, or iodo, and a base in an inert solvent to provide the novel sulfonamide (O) of the present invention. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), alcohol solvents (preferably 2-propanol, etc.), or aromatic solvents (preferably toluene or pyridine, etc.). Reaction temperature ranges from about −20° C. to 50° C., preferably about 0° C. to 40° C.

The novel urea (P) or thiourea (P) of the present invention can be obtained by urea reaction or thiourea reaction using an isocyanate (R₁NCO) or isothiocyanate (R₁NCS) in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine or imidazole, etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferably about 0° C. to 100° C.

The novel urethane (Q) of the present invention can be obtained by urethane reaction using R₁OCOX, wherein X is halogen such as chloro, bromo, or iodo, in an inert solvent with or without a base. The base includes an alkali metal carbonate (preferably sodium carbonate or potassium carbonate, etc.), an alkali metal hydrogencarbonate (preferably sodium hydrogencarbonate or potassium hydrogencarbonate, etc.), an alkali hydroxide (preferably sodium hydroxide or potassium hydroxide, etc.), a tertiary amine (preferably N,N-diisopropylethylamine, triethylamine, or N-methylmorpholine, etc.), or an aromatic amine (preferably pyridine, imidazole, or poly-(4-vinylpyridine), etc.). The inert solvent includes lower halocarbon solvents (preferably dichloromethane, dichloroethane, or chloroform, etc.), ethereal solvents (preferably tetrahydrofuran or dioxane), aromatic solvents (preferably benzene or toluene, etc.), or polar solvents (preferably N,N-dimethylformamide or dimethyl sulfoxide, etc.). Reaction temperature ranges from about −20° C. to 120° C., preferably about 0° C. to 100° C.

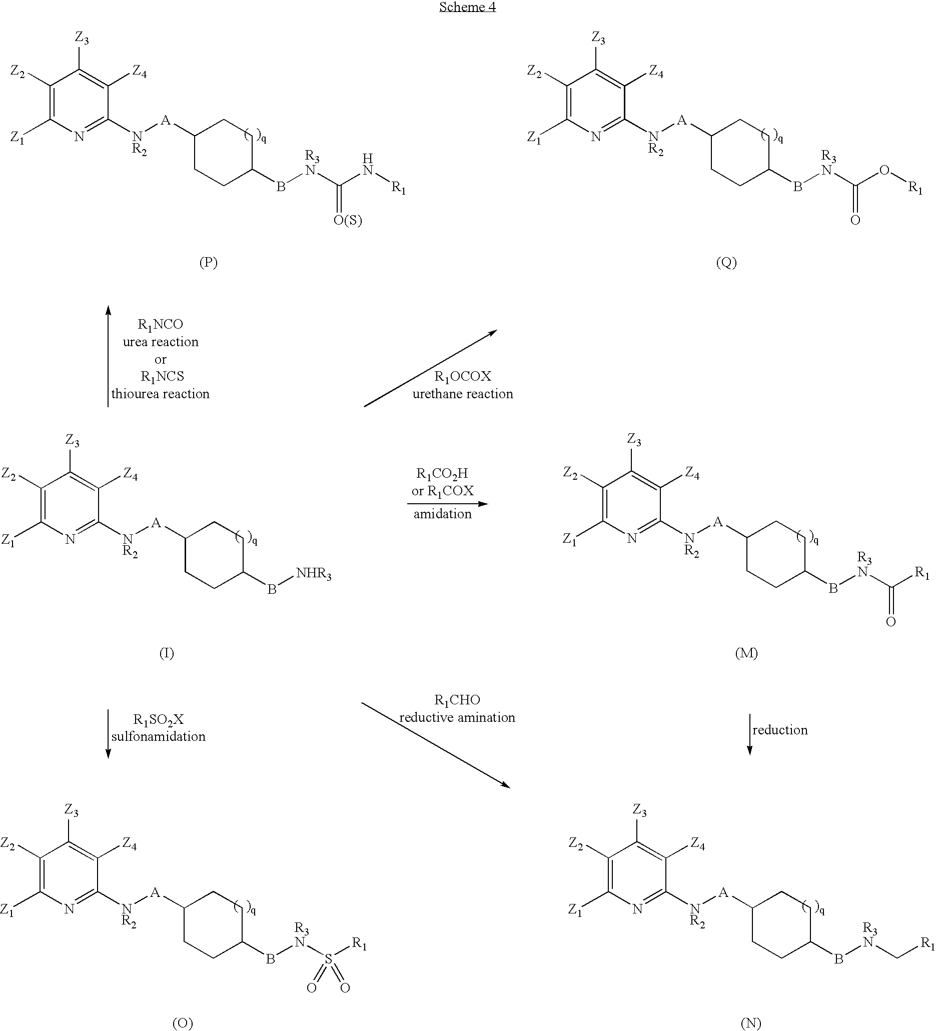

Also the novel 2,4-diamino-substituted pyridine (U) of the present invention can be prepared as shown in Scheme 5.

First 2-halo-4-nitro-pyridine N-oxide (B), which is synthesized in Scheme 1, is substituted by the amine (R) which has been already installed by the desired R₁ substituent, wherein R₂, R₃, A, B, Y, and R₁ are as defined above, with or without a base in an inert solvent to provide the coupling adduct (S). The base includes an alkali metal carbonate (preferably sodium carbonate or potassium carbonate, etc.), an alkali metal hydroxide (preferably sodium hydroxide, etc.), or a tertiary amine (preferably N,N-diisopropylethylamine, triethylamine, or N-methylmorpholine, etc.). The inert solvent includes lower alkyl alcohol solvents (preferably methanol, ethanol, 2-propanol, or butanol, etc.) or amide solvents (preferably N,N-dimethylformamide or 1-methyl-pyrrolidin-2-one, etc.). Reaction temperature ranges from about 50° C. to 200° C., preferably about 80° C. to 150° C. Also this reaction can be carried out under microwave conditions. Reduction of a nitro group and N-oxide can be achieved by the same method as shown Scheme 2. Alkylation of the amino group leads to the novel 2,4-diamino-substituted pyridine (U) of the present invention. The amino group can be alkylated by forming an amide group and reducing the amide as indicated in Scheme 4. Another method of alkylation comprises reaction of the amine with an aldehyde and reduction of the resulting Schiff's base either in situ or after its isolation as indicated in Scheme 4. A further method of alkylation comprises condensation of the amino group with a reagent such as R₄ₐ-L, wherein R₄ₐ is as defined above and L is a leaving group, for instance, halogen or a sulphonyloxy group such as methylsulphonyloxy or 4-toluenesulphonyloxy. The condensation can be carried out in the presence of a base, for instance, sodium hydride, cesium carbonate, potassium carbonate, diisopropylethylamine, or triethylamine, etc. Reaction temperature ranges from about 50° C. to 200° C., preferably about 80° C. to 150° C.

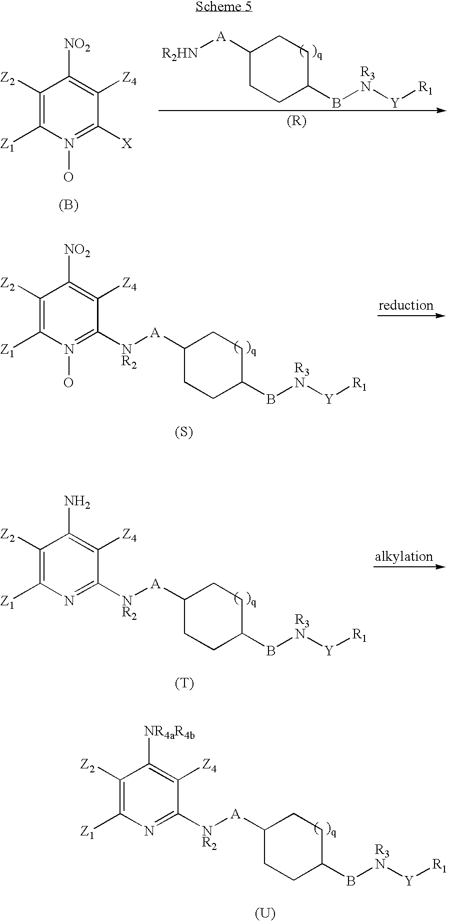

Alternatively, the novel pyridines (Y) of the present invention are directly synthesized from the pyridine (X) as shown in Scheme 6.

2-Hydroxypyridin (V) and 2-pyridone (W) which are commercially available or synthesized by a well known method, wherein Z₁, Z₂, Z₃ and Z₄ are as defined above, are halogenated by the method as shown Scheme 1 to provide the 2-halopyridine (X). Coupling of the 2-halopyridine (X) with the amine (R), which has been already installed by the desired R₁ substituent, wherein R₂, R₃, A, B, Y, and R₁ are as defined above, is accomplished by the conditions as indicated in Scheme 5 to give the novel pyridines (Y) of the present invention. Also the 2-halopyridine (X) could be synthesized from the pyridine (Z), which is oxidized to the pyridine N-oxide (A′) by the same method as shown Scheme 1. Halogenation of the pyridine N-oxide (A′) is achieved by the same manner as shown Scheme 1 to provide the 2-halopyridine (X).

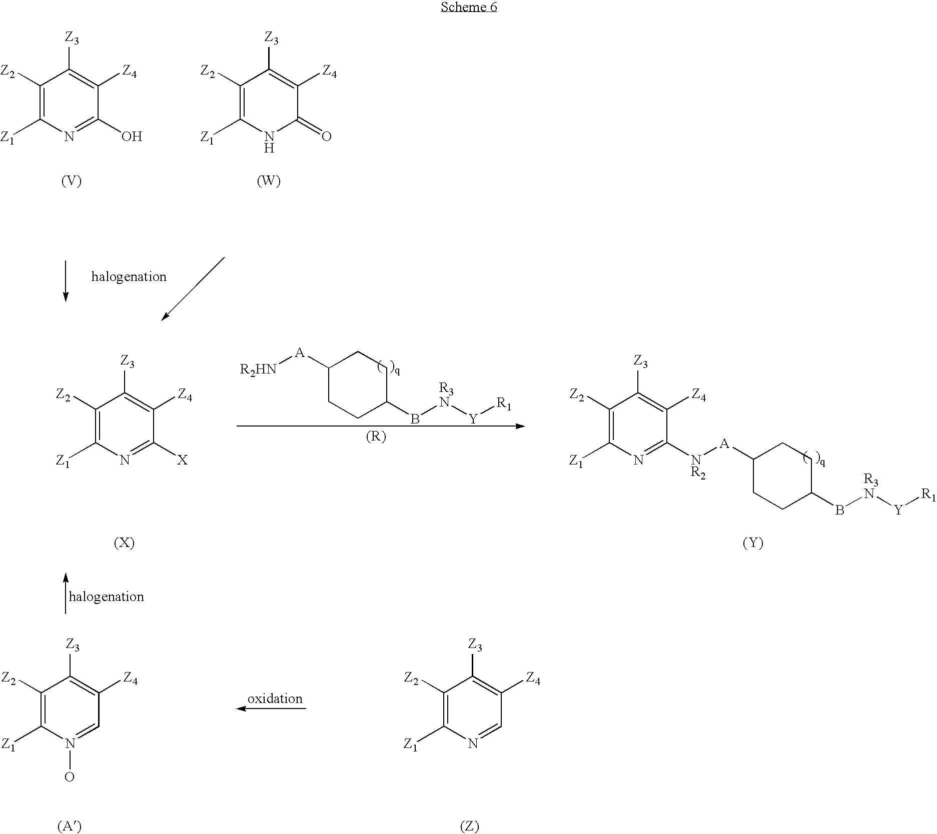

### EXAMPLES

The compounds of the invention and their synthesis are further illustrated by the following examples. The following examples are provided to further define the invention without, however, limiting the invention to the particulars of these examples. “Ambient temperature” as referred to in the following example is meant to indicate a temperature falling between 0° C. and 40° C. The following compounds are named by ACD Name Version 7.0.

Abbreviations used in the instant specification, particularly the Schemes and Examples, are as follows:

¹H NMR: proton nuclear magnetic resonance spectrum

AcCl: acetyl chloride

Ac₂O: acetic anhydride

AcOH: acetic acid

AcONH₄: ammonium acetate

APCI: atmospheric pressure chemical ionization

BuOH: butanol

Cbz: carbobenzoxy

CDCl₃: deuterated chloroform

CHCl₃: chloroform

CI: chemical ionization

Conc.: concentrated

DMF: N,N-dimethylformamide

DMSO: dimethyl sulfoxide

EDC: 1-[3-(dimethylamino)propyl]-3-ethylcarbodiimide

EI: electron ionization

ESI: electrospray ionization

Et₃N: triethylamine

Et₂O: diethyl ether

EtOAc: acetic acid ethyl ester

EtOH: ethanol

FAB: fast atom bombardment

Fe: iron

H₂O: water

H₂O₂: hydrogen peroxide

H₂SO₄: sulfuric acid

HCl: hydrogen chloride

HNO₃: nitric acid

HOBt: 1-hydroxybenzotriazole

K₂CO₃: potassium carbonate

mCPBA: m-chloroperbenzoic acid

MS: mass spectrometry

Me₂NH: dimethylamine

MeNH₂: methylamine

MeOH: methanol

MgSO₄: magnesium sulfate

NH₃: ammonia

NaBH(OAc)₃: sodium triacetoxyborohydride

NaBH₃CN: sodium cyanoborohydride

NaBH₄: sodium borohydride

NaHCO₃: sodium hydrogencarbonate

Na₂SO₄: sodium sulfate

Na₂S₂O₃: sodium thiosulfate

Pd/C: palladium carbon

POCl₃: phosphoryl chloride

SOCl₂: thionyl chloride

TFA: trifluoroacetic acid

THF tetrahydrofuran

ZCl: benzyloxycarbonyl chloride

s: singlet

d: doublet

t triplet

q: quartet

dd: doublet doublet

dt: doublet triplet

ddd: doublet doublet doublet

brs: broad singlet

m: multiplet

J: coupling constant

Hz: Hertz

### Example 1

### 3-Chloro-4-fluoro-N-[cis-4-(pyridin-2-ylamino)cyclohexyl]benzamide hydrochloride

### Step A: Synthesis of N-(cis-4-aminocyclohexyl)-3-chloro-4-fluorobenzamide

To a solution of tert-butyl (cis-4-aminocyclohexyl)carbamate (130 g) in DMF (1.3 L) were added 3-chloro-4-fluorobenzoic acid (116 g), Et₃N (202 mL), HOBt-H₂O (139 g), and EDC-HCl (128 g). The mixture was stirred at ambient temperature for 16 h. To the mixture was added water (3 L) and the suspension was stirred at ambient temperature for 4 h. The precipitate was collected by filtration, washed with water, and dried at 80° C. under reduced pressure to give a brown solid (216 g). To a suspension of the above solid in EtOAc (1.3 L) was added 4 M hydrogen chloride in EtOAc (1.3 L) under 10° C. and the mixture was stirred at ambient temperature for 12 h. The precipitate was collected by filtration, washed with EtOAc, and dried at 80° C. under reduced pressure to give a pale brown solid (174 g). To the above solid was added 1 M aqueous NaOH (1 L). The mixture was stirred at ambient temperature for 30 min and poured into CHCl₃. The aqueous layer was extracted with CHCl₃ three times. The combined organic layer was dried over MgSO₄, filtered, and concentrated under reduced pressure to give N-(cis-4-aminocyclohexyl)-3-chloro-4-fluorobenzamide (145 g) as a pale brown solid.

¹H NMR (300 MHz, CDCl₃, δ): 1.26-1.54 (m, 4H), 1.65-1.92 (m, 6H), 2.94-3.07 (m, 1H), 4.07-4.22 (m, 1H), 5.98-6.17 (m, 1H), 7.19 (t, J=8.6 Hz, 1H), 7.60-7.68 (m, 1H), 7.83 (dd, J=6.9, 2.3 Hz, 1H); ESI MS m/z 271 (M⁺+1, 100%).

### Step B: Synthesis of 3-chloro-4-fluoro-N-[cis-4-(pyridin-2-ylamino)cyclohexyl]benzamide hydrochloride

A mixture of N-(cis-4-aminocyclohexyl)-3-chloro-4-fluorobenzamide (2.62 g), 2-chloropyridine (1.00 g), and BuOH (1 mL) was heated in a microwave synthesizer at 230° C. for 20 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 20% to 66% EtOAc in hexane). To a solution of the above purified material in EtOAc (10 mL) was added 4 M hydrogen chloride in EtOAc (4.4 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-[cis-4-(pyridin-2-ylamino)cyclohexyl]benzamide hydrochloride (317 mg) as a white solid.

¹H NMR (300 MHz, CDCl₃, δ): 1.73-2.05 (m, 8H), 3.77-3.90 (m, 1H), 4.05-4.22 (m, 1H), 6.62-6.88 (m, 3H), 7.18 (t, J=8.6 Hz, 1H), 7.66-7.89 (m, 3H), 7.95 (dd, J=7.0, 2.2 Hz, 1H), 9.08-9.25 (m, 1H); ESI MS m/z 348 [(M(free)⁺+1, 100%].

### Example 2

### 3-Chloro-4-fluoro-N-{cis-4-[(5-methylpyridin-2-yl)amino]cyclohexyl}benzamide hydrochloride

Using the procedure for the step B of example 1, the title compound was obtained.

¹H NMR (300 MHz, CDCl₃, δ): 1.70-2.04 (m, 8H), 2.25 (s, 3H), 3.75-3.88 (m, 1H), 4.05-4.23 (m, 1H), 6.69-6.82 (m, 2H), 7.18 (t, J=8.6 Hz, 1H), 7.54 (s, 1H), 7.63-7.76 (m, 2H), 7.95 (dd, J=7.0, 2.2 Hz, 1H), 8.84-9.00 (m, 1H); ESI MS m/z 362 [M(free)⁺+1, 100%].

### Example 3

### 3-Chloro-4-fluoro-N-{cis-4-[(6-methylpyridin-2-yl)amino]cyclohexyl}benzamide hydrochloride

Using the procedure for the step B of example 1, the title compound was obtained.

¹H NMR (300 MHz, CDCl₃, δ): 1.72-2.03 (m, 8H), 2.57 (s, 3H), 3.76-3.87 (m, 1H), 4.06-4.21 (m, 1H), 6.52 (d, J=7.3 Hz, 1H), 6.61 (d, J=9.0 Hz, 1H), 6.72-6.82 (m, 1H), 7.18 (t, J=8.6 Hz, 1H), 7.67-7.76 (m, 2H), 7.95 (dd, J=7.0, 2.2 Hz, 1H), 8.90-9.00 (m, 1H); ESI MS m/z 362 [M (free)⁺+1, 100%].

### Example 4

### 3-Chloro-N-(cis-4-{[4-(dimethylamino)-5-methylpyridin-2-yl]amino}cyclohexyl)-4-fluorobenzamide hydrochloride

### Step A: Synthesis of 2-chloro-5-methyl-4-nitro-pyridine 1-oxide

To a suspension of 2-chloro-5-methylpyridine (10.0 g) in Ac₂O (25.0 mL) was added 30% aqueous H₂O₂ (25.0 mL). The mixture was stirred at ambient temperature for 24 h and 60° C. for 30 h. To the mixture was added 2 M aqueous NaOH (200 mL) and the aqueous layer was extracted with CHCl₃ three times. The combined organic layer was dried over MgSO₄, filtered, and concentrated under reduced pressure. The residue was dissolved in concentrated H₂SO₄ (15.0 mL) and the solution was poured into mixture of concentrated H₂SO₄ (15.0 mL) and fuming HNO₃ (25.0 mL). The mixture was stirred at 100° C. for 30 min and poured into ice (500 mL). The solution was alkalized with ammonium hydrogencarbonate (pH=9) and 28% aqueous NH₃ (pH=13). The mixture was stirred at ambient temperature for 2 h and the precipitate was collected by filtration, washed with H₂O and hexane, and dried at 50° C. under reduced pressure to give 2-chloro-5-methyl-4-nitro-pyridine 1-oxide (10.6 g) as a pale yellow solid.

¹H NMR (300 MHz, CDCl₃, δ): 2.61 (s, 3H), 8.25-8.30 (m, 2H); CI MS m/z 189 (M⁺+1, 100%).

### Step B: Synthesis of 2,4-dichloro-5-methylpyridine 1-oxide

A mixture of 2-chloro-5-methyl-4-nitro-pyridine 1-oxide (7.00 g) and AcCl (35.0 mL) was stirred at reflux for 1 h. The mixture was poured into ice and alkalized with aqueous saturated NaHCO₃ (pH=8). The aqueous layer was extracted with CHCl₃ three times. The combined organic layer was dried over MgSO₄, filtered, and concentrated under reduced pressure to give 2,4-dichloro-5-methylpyridine 1-oxide (5.68 g) as a white solid.

¹H NMR (300 MHz, CDCl₃, δ): 2.31 (s, 3H), 7.49 (s, 1H), 8.23 (s, 1H); ESI MS m/z 178 (M⁺+1, 30%), 200 (M⁺+23, 100%)

### Step C: Synthesis of cis-N-benzylcyclohexane-1,4-diamine

To a solution of tert-butyl (cis-4-aminocyclohexyl)carbamate (380 g) in CHCl₃ (3 L) were added benzaldehyde (188 g) and AcOH (106 mL). After the mixture was stirred at ambient temperature for 1 h, NaBH(OAc)₃ (450 g) was added. The mixture was stirred at ambient temperature for 14 h. To the mixture was added 1M aqueous NaOH (1 L) and the aqueous layer was extracted with CHCl₃ three times. The combined organic layer was dried over MgSO₄, filtered, and concentrated under reduced pressure. The residue was suspended in 50% EtOAc in hexane. The suspension was stirred at ambient temperature for 1 h and the precipitate was collected by filtration and dried under reduced pressure to give a pale brown oil (690 g). To the suspension of the above oil (690 g) in EtOAc (2.07 L) was added 4 M hydrogen chloride in EtOAc (1.7 L) and stirred at ambient temperature for 2 h. The precipitate was collected by filtration, washed with EtOAc, and dissolved in 1 M aqueous NaOH. The solution was stirred at ambient temperature for 5 min and the aqueous layer was extracted with CHCl₃ three times. The combined organic layer was dried over MgSO₄, filtered, and concentrated under reduced pressure to give cis-N-benzylcyclohexane-1,4-diamine (292 g) as a pale orange oil.

¹H NMR (200 MHz, CDCl₃, δ): 1.44-1.74 (m, 8H), 2.62-2.76 (m, 1H), 2.77-2.95 (m, 1H), 3.77 (s, 2H), 7.14-7.44 (m, 5H); ESI MS m/z 205 (M>⁺+1, 100%).

### Step D: Synthesis of cis-N-benzyl-N′-(4-chloro-5-methyl-1-oxidopyridin-2-yl) cyclohexane-1,4-diamine

A mixture of 2,4-dichloro-5-methylpyridine 1-oxide (2.10 g), cis-N-benzylcyclohexane-1,4-diamine (3.67 g), and BuOH (4 mL) was stirred at reflux for 4 h. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 25% to 100% EtOAc in hexane) to give cis-N-benzyl-N′-(4-chloro-5-methyl-1-oxidopyridin-2-yl)cyclohexane-1,4-diamine (2.77 g) as a brown oil.

¹H NMR (300 MHz, CDCl₃, δ): 1.50-1.98 (m, 8H), 2.18 (s, 3H), 2.69-2.80 (m, 1H), 3.38-3.52 (m, 1H), 3.79 (s, 2H), 6.57 (s, 1H), 6.74-6.83 (m, 1H), 7.20-7.36 (m, 5H), 7.99 (s, 1H); ESI MS m/z 346 (M⁺+1, 100%).

### Step E: Synthesis of N²-[cis-4-(benzylamino)cyclohexyl]-N⁴,N⁴,5-trimethylpyridine-2,4-diamine 1-oxide

A mixture of cis-N-benzyl-N′-(4-chloro-5-methyl-1-oxidopyridin-2-yl)cyclohexane-1,4-diamine (917 mg), 2 M Me₂NH in MeOH (2.65 mL), and BuOH (0.67 mL) was heated in a microwave synthesizer at 160° C. for 3.5 h. The reaction was repeated 2 more times and the reaction mixtures were pooled. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane) to give N²-[cis-4-(benzylamino)cyclohexyl]-N⁴,N⁴,5-trimethylpyridine-2,4-diamine 1-oxide (1.12 g) as a brown oil.

¹H NMR (300 MHz, CDCl₃, δ): 1.43-1.97 (m, 8H), 2.12 (s, 3H), 2.67-2.86 (m, 7H), 3.40-3.61 (m, 1H), 3.80 (s, 2H), 5.96 (s, 1H), 6.64-6.78 (m, 1H), 7.21-7.36 (m, 5H), 7.81 (s, 1H); ESI MS m/z 355 (M⁺+1, 100%).

### Step F: Synthesis of N²-(cis-4-aminocyclohexyl)-N⁴,N⁴,5-trimethylpyridine-2,4-diamine

A mixture of N²-[cis-4-(benzylamino)cyclohexyl]-N⁴,N⁴,5-trimethylpyridine-2,4-diamine 1-oxide (1.10 g), cyclohexene (6.16 mL), 10% palladium carbon (765 mg), and 2-propanol (24.0 mL) was stirred at reflux for 76 h. The mixture was filtered through a pad of celite, concentrated under reduced pressure, and purified by medium-pressure liquid chromatography (NH-silica gel, 2% to 5% MeOH in CHCl₃) to give N²-(cis-4-aminocyclohexyl)-N⁴,N⁴,5-trimethylpyridine-2,4-diamine (398 mg) as a brown oil.

¹H NMR (200 MHz, CDCl₃, δ): 1.23-1.94 (m, 8H), 2.13 (s, 3H), 2.69-2.97 (m, 7H), 3.67-3.86 (m, 1H), 4.28-4.49 (m, 1H), 5.82 (s, 1H), 7.71 (s, 1H); ESI MS m/z 249 (M⁺+1, 85%), 334 (M⁺+86, 100%).

### Step G: Synthesis of 3-chloro-N-(cis-4-{[4-(dimethylamino)-5-methylpyridin-2-yl]amino}cyclohexyl)-4-fluorobenzamide hydrochloride

To a solution of N²-(cis-4-aminocyclohexyl)-N⁴,N⁴,5-trimethylpyridine-2,4-diamine (195 mg) in DMF (3.2 mL) were added 3-chloro-4-fluorobenzoic acid (164 mg), Et₃N (260 μL), HOBt-H₂O (179 mg), and EDC-HCl (180 mg). The mixture was stirred at ambient temperature for 12 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 67% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-N-(cis-4-{[4-(dimethylamino)-5-methylpyridin-2-yl]amino}cyclohexyl)-4-fluorobenzamide hydrochloride (227 mg) as a white solid.

¹H NMR (300 MHz, CDCl₃, δ): 1.65-2.07 (m, 8H), 2.21 (s, 3H), 3.06 (s, 6H), 3.66-3.79 (m, 1H), 4.02-4.22 (m, 1H), 5.66 (s, 1H), 6.75-6.91 (m, 1H), 7.09-7.27 (m, 2H), 7.68-7.79 (m, 1H), 7.96 (d, J=8.6 Hz, 1H), 8.21-8.34 (m, 1H), 13.12-13.35 (m, 1H); ESI MS m/z 405 [M (free)⁺+1, 100%].

### Example 5

### N-(cis-4-{[4-(Dimethylamino)-5-methylpyridin-2-yl]amino}cyclohexyl)-3,4,5-trifluorobenzamide hydrochloride

Using the procedure for the step G of example 4, the title compound was obtained.

¹H NMR (300 MHz, CDCl₃, δ): 1.63-2.11 (m, 8H), 2.21 (s, 3H), 3.07 (s, 6H), 3.68-3.80 (m, 1H), 4.02-4.20 (m, 1H), 5.66 (s, 1H), 7.05-7.13 (m, 1H), 7.23 (s, 1H), 7.51-7.62 (m, 2H), 8.19-8.28 (m, 1H); ESI MS m/z 407 [M (free)⁺+1, 100%].

### Example 6

### 3-Chloro-N-(cis-4-{[4-(dimethylamino)-5-methylpyridin-2-yl]amino}cyclohexyl)-5-fluorobenzamide hydrochloride

Using the procedure for the step G of example 4, the title compound was obtained.

¹H NMR (300 MHz, CDCl₃, δ): 1.66-2.03 (m, 8H), 2.21 (s, 3H), 3.06 (s, 6H), 3.67-3.79 (m, 1H), 4.02-4.19 (m, 1H), 5.65 (s, 1H), 6.95 (d, J=8.7 Hz, 1H), 7.15-7.26 (m, 2H), 7.44-7.52 (m, 1H), 7.64 (s, 1H), 8.28 (d, J=7.8 Hz, 1H), 13.20-13.30 (m, 1H); ESI MS m/z 405 [M (free)⁺+1, 100%].

### Example 7

### 3,5-Dichloro-N-(cis-4-{[4-(dimethylamino)-5-methylpyridin-2-yl]amino}cyclohexyl)benzamide hydrochloride

To a solution of N²-(cis-4-aminocyclohexyl)-N⁴,N⁴,5-trimethylpyridine-2,4-diamine obtained in step F of example 4 (250 mg) in CHCl₃ (3 mL) were added Et₃N (293 μL) and 3,5-dichlorobenzoyl chloride (272 mg). The mixture was stirred at ambient temperature for 14 h and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 14% to 33% EtOAc in hexane). To a solution of the above purified material in EtOAc (10 mL) was added 4 M hydrogen chloride in EtOAc (0.25 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3,5-dichloro-N-(cis-4-{[4-(dimethylamino)-5-methylpyridin-2-yl]amino}cyclohexyl)benzamide hydrochloride (69 mg) as a white solid.

¹H NMR (300 MHz, CDCl₃, δ): 1.65-2.03 (m, 8H), 2.21 (s, 3H), 3.06 (s, 6H), 3.66-3.78 (m, 1H), 4.03-4.19 (m, 1H), 5.65 (s, 1H), 6.93-7.01 (m, 1H), 7.21-7.26 (m, 1H), 7.42-7.48 (m, 1H), 7.70-7.76 (m, 2H), 8.20-8.33 (m, 1H), 13.09-13.34 (m, 1H); ESI MS m/z 421 [M (free)⁺+1, 100%].

### Example 8

### N-(cis-4-{[4-(Dimethylamino)-5-methylpyridin-2-yl]amino}cyclohexyl)-3,4-difluorobenzamide hydrochloride

Using the procedure for the step G of example 4, the title compound was obtained.

¹H NMR (300 MHz, CDCl₃, δ): 1.67-2.10 (m, 8H), 2.21 (s, 3H), 3.06 (s, 6H), 3.68-3.78 (m, 1H), 4.03-4.19 (m, 1H), 5.66 (s, 1H), 6.88 (d, J=8.7 Hz, 1H), 7.14-7.26 (m, 2H), 7.56-7.65 (m, 1H), 7.68-7.80 (m, 1H), 8.25 (d, J=8.4 Hz, 1H), 13.20 (brs, 1H); ESI MS m/z 389 [M (free)⁺+1, 100%].

### Example 9

### 3,4-Dichloro-N-(cis-4-{[4-(Dimethylamino)-5-methylpyridin-2-yl]amino}cyclohexyl)benzamide hydrochloride

Using the procedure for the step G of example 4, the title compound was obtained.

¹H NMR (300 MHz, CDCl₃, δ): 1.67-2.15 (m, 8H), 2.21 (s, 3H), 3.06 (s, 6H), 3.67-3.78 (m, 1H), 4.02-4.19 (m, 1H), 5.65 (s, 1H), 6.90 (d, J=8.9 Hz, 1H), 7.23 (brs, 1H), 7.48 (d, J=8.2 Hz, 1H), 7.65-7.71 (m, 1H), 7.99 (d, J=2.0 Hz, 1H), 8.21 (d, J=8.1 Hz, 1H), 13.12 (brs, 1H); ESI MS m/z 421 [M (free)⁺+1, 100%].

### Example 10

### 3-Chloro-N-(cis-4-{[4-(dimethylamino)-5-methylpyridin-2-yl]amino}cyclohexyl)benzamide hydrochloride

Using the procedure for the step G of example 4, the title compound was obtained.

¹H NMR (300 MHz, CDCl₃, δ): 1.67-2.02 (m, 8H), 2.21 (s, 3H), 3.06 (s, 6H), 3.64-3.78 (m, 1H), 4.05-4.22 (m, 1H), 5.66 (s, 1H), 6.83 (d, J=8.4 Hz, 1H), 7.22-7.24 (m, 1H), 7.31-7.47 (m, 2H), 7.67-7.72 (m, 1H), 7.84-7.86 (m, 1H), 8.28 (d, J=8.1 Hz, 1H); ESI MS m/z 387 [M (free)⁺+1, 100%].

### Example 11

### 5-Bromo-N-(cis-4-{[4-(dimethylamino)-5-methylpyridin-2-yl]amino}cyclohexyl)nicotinamide dihydrochloride

Using the procedure for the step G of example 4, the title compound was obtained.

¹H NMR (300 MHz, CDCl₃, δ): 1.64-2.13 (m, 8H), 2.22 (s, 3H), 3.06 (s, 6H), 3.68-3.80 (m, 1H), 4.09-4.22 (m, 1H), 5.74 (brs, 1H), 6.91-7.51 (m, 2H), 8.84-8.98 (m, 1H), 9.07-9.16 (m, 2H), 9.68 (brs, 1H), 12.85 (brs, 1H); ESI MS m/z 432 [M (free)⁺+1, 100%], 434 [M (free)⁺+3, 100%].

### Example 12

### N-(cis-4-{[4-(Dimethylamino)-5-methylpyridin-2-yl]amino}cyclohexyl)-3-(trifluoromethoxy)-benzamide hydrochloride

Using the procedure for the step G of example 4, the title compound was obtained.

¹H NMR (300 MHz, CDCl₃, δ): 1.67-2.04 (m, 8H), 2.21 (s, 3H), 3.06 (s, 6H), 3.68-3.77 (m, 1H), 4.07-4.22 (m, 1H), 5.66 (s, 1H), 6.83 (d, J=8.2 Hz, 1H), 7.21-7.36 (m, 2H), 7.42-7.49 (m, 1H), 7.71-7.76 (m, 2H), 8.29 (d, J=8.2 Hz, 1H); ESI MS m/z 437 [M (free)⁺+1, 100%].

### Example 13

### N-(cis-4-{[4-(Dimethylamino)-5-methylpyridin-2-yl]amino}cyclohexyl)-4-fluoro-3-(trifluoromethyl)benzamide hydrochloride

Using the procedure for the step G of example 4, the title compound was obtained.

¹H NMR (300 MHz, CDCl₃, δ): 1.66-2.08 (m, 8H), 2.21 (s, 3H), 3.07 (s, 6H), 3.69-3.79 (m, 1H), 4.06-4.23 (m, 1H), 5.66 (s, 1H), 6.99-7.08 (m, 1H), 7.20-7.29 (m, 2H), 8.02-8.10 (m, 1H), 8.18-8.29 (m, 2H); ESI MS m/z 439 [M (free)⁺+1, 100%].

### Example 14

### N-(cis-4-{[4-(Dimethylamino)-5-methylpyridin-2-yl]amino}cyclohexyl)-3-(trifluoromethyl)-benzamide hydrochloride

Using the procedure for the step G of example 4, the title compound was obtained.

¹H NMR (300 MHz, CDCl₃, δ): 1.69-2.05 (m, 8H), 2.21 (s, 3H), 3.06 (s, 6H), 3.68-3.79 (m, 1H), 4.07-4.22 (m, 1H), 5.66 (s, 1H) 6.97 (d, J=8.6 Hz, 1H), 7.23 (s, 1H), 7.50-7.60 (m, 1H), 7.68-7.76 (m, 1H), 8.01 (d, J=7.8 Hz, 1H), 8.16 (s, 1H), 8.27 (d, J=8.4 Hz, 1H); ESI MS m/z 421 [M (free)⁺+1, 100%].

### Example 15

### 3-Chloro-4-fluoro-N-(cis-4-{[5-methyl-4-(methylamino)pyridin-2-yl]amino}cyclohexyl)-benzamide hydrochloride

### Step A: Synthesis of 3-chloro-4-fluoro-N-{cis-4-[(5-methyl-4-nitro-1-oxidopyridin-2-yl)amino]-cyclohexyl}benzamide

A mixture of 2-chloro-5-methyl-4-nitro-pyridine 1-oxide obtained in step A of example 4 (3.00 g), N-(cis-4-aminocyclohexyl)-3-chloro-4-fluorobenzamide obtained in step A of example 1 (4.74 g), and BuOH (6 mL) was stirred at reflux for 8 h. The mixture was poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (silica gel, 50% to 100% EtOAc in hexane) to give 3-chloro-4-fluoro-N-{cis-4-[(5-methyl-4-nitro-1-oxidopyridin-2-yl)amino]cyclohexyl}benzamide (2.33 g) as a yellow solid.

¹H NMR (300 MHz, DMSO-d₆, δ): 1.61-1.96 (m, 8H), 2.35 (s, 3H), 3.79-4.04 (m, 2H), 6.95 (d, J=8.7 Hz, 1H), 7.47-7.54 (m, 1H), 7.86-7.94 (m, 1H), 8.09 (dd, J=7.3, 2.2 Hz, 1H), 8.29-8.35 (m, 1H), 8.43 (s, 1H); ESI MS m/z 178 (M⁺−244, 100%), 180 (M⁺−242, 65%), 423 (M⁺+1, 50%).

### Step B: Synthesis of N-{cis-4-[(4-amino-5-methylpyridin-2-yl)amino]cyclohexyl}-3-chloro-4-fluorobenzamide

A mixture of 3-chloro-4-fluoro-N-{cis-4-[(5-methyl-4-nitro-1-oxidopyridin-2-yl)amino]-cyclohexyl}benzamide (2.19 g), Fe (4.38 g), and AcOH (44 mL) was stirred at 100° C. for 15 min. The mixture was filtered through a pad of celite and the filtrate was poured into 1 M aqueous NaOH. The aqueous layer was extracted with EtOAc three times. The combined organic layer was dried over MgSO₄, filtered, concentrated under reduced pressure, and purified by medium-pressure liquid chromatography (NH-silica gel, 50% to 100% EtOAc in hexane) to give N-{cis-4-[(4-amino-5-methylpyridin-2-yl)amino]cyclohexyl}-3-chloro-4-fluorobenzamide (1.33 g) as a colorless solid.

¹H NMR (300 MHz, CDCl₃, δ): 1.55-1.93 (m, 8H), 2.00 (s, 3H), 3.57-3.72 (m, 1H), 3.91-4.19 (m, 3H), 4.30-4.45 (m, 1H), 5.67 (s, 1H), 6.08-6.21 (m, 1H), 7.19 (t, J=8.6 Hz, 1H), 7.60-7.70 (m, 2H), 7.85 (dd, J=7.0, 2.2 Hz, 1H); ESI MS m/z 377 (M⁺+1, 100%).

### Step C: Synthesis of 3-chloro-4-fluoro-N-(cis-4-{[5-methyl-4-(methylamino)pyridin-2-yl]amino}cyclohexyl)benzamide hydrochloride

To a solution of N-{cis-4-[(4-amino-5-methylpyridin-2-yl)amino]cyclohexyl}-3-chloro-4-fluorobenzamide (630 mg) in AcOH (16 mL) was added paraformaldehyde (56.0 mg). The mixture was stirred at ambient temperature for 1.5 h and NaBH₃CN (116 mg) was added. The mixture was stirred at ambient temperature for 10 h. To the mixture were added paraformaldehyde (56.0 mg) and NaBH₃CN (116 mg). The mixture was stirred at ambient temperature for 5 h and poured into 1 M aqueous HCl. The aqueous layer was extracted with CHCl₃ three times. The combined organic layer was dried over MgSO₄, filtered, concentrated under reduced pressure, and purified by medium-pressure liquid chromatography (silica gel, 5% to 15% MeOH in CHCl₃) to give a colorless solid. To a solution of the above solid in EtOAc (5 mL) was added 4 M hydrogen chloride in EtOAc (10 mL). The mixture was stirred at ambient temperature for 1 h and concentrated under reduced pressure. A suspension of the residue in Et₂O (20 mL) was stirred at ambient temperature for 4 h. The precipitate was collected by filtration, washed with Et₂O, and dried at 80° C. under reduced pressure to give 3-chloro-4-fluoro-N-(cis-4-{[5-methyl-4-(methylamino)pyridin-2-yl]amino}cyclohexyl)benzamide hydrochloride (88.0 mg) as a white solid.

¹H NMR (300 MHz, CDCl₃, δ): 1.65-2.14 (m, 11H), 3.00 (d, J=3.7 Hz, 3H), 3.68-3.82 (m, 1H), 4.03-4.20 (m, 1H), 5.05 (brs, 1H), 5.48 (s, 1H), 6.91 (d, J=8.6 Hz, 1H), 7.16-7.23 (m, 2H), 7.70-7.79 (m, 1H), 7.97 (dd, J=7.0, 2.2 Hz, 1H), 8.22 (d, J=7.9 Hz, 1H), 12.89 (brs, 1H); ESI MS m/z 391 [M (free)⁺+1, 100%].

### Example 16

### 3,4,5-Trifluoro-N-(cis-4-{[5-methyl-4-(methylamino)pyridin-2-yl]amino}cyclohexyl)-benzamide hydrochloride

### Step A: Synthesis of N-(cis-4-aminocyclohexyl)-3,4,5-trifluorobenzamide

To a solution of tert-butyl (cis-4-aminocyclohexyl)carbamate (44.3 g) in DMF (450 mL) were added 3,4,5-trifluorobenzoic acid (40.1 g), Et₃N (69.2 mL), HOBt-H₂O (47.5 g), and EDC-HCl (43.6 g). The mixture was stirred at ambient temperature for 12 h. To the mixture was added water (1 L) and the suspension was stirred at ambient temperature for 2 h. The precipitate was collected by filtration, washed with water and hexane, and dried at 80° C. under reduced pressure to give a pale brown solid (82.7 g). To the suspension of the above solid in EtOAc (800 mL) was added 4 M hydrogen chloride in EtOAc (600 mL) under 10° C. The mixture was stirred at ambient temperature for 6 h and concentrated under reduced pressure. The residue was dissolved in CHCl₃ (300 mL) and poured into 1 M aqueous NaOH (500 mL). The aqueous layer was extracted with CHCl₃ three times. The combined organic layer was dried over MgSO₄, filtered, concentrated under reduced pressure to give N-(cis-4-aminocyclohexyl)-3,4,5-trifluorobenzamide (65.3 g) as a pale brown solid.

¹H NMR (300 MHz, CDCl₃, δ): 1.38-1.91 (m, 8H), 2.97-3.09 (m, 1H), 4.04-4.20 (m, 1H), 6.15-6.27 (m, 1H), 7.35-7.50 (m, 2H); ESI MS m/z 273 (M⁺+1, 100%).

### Step B: Synthesis of 3,4,5-trifluoro-N-{cis-4-[(5-methyl-4-nitro-1-oxidopyridin-2-yl)amino]cyclohexyl}benzamide

A mixture of 2-chloro-5-methyl-4-nitro-pyridine 1-oxide obtained in step A of example 4 (3.00 g), N-(cis-4-aminocyclohexyl)-3,4,5-trifluorobenzamide (4.76 g), and BuOH (6 mL) was stirred at reflux for 8 h. The mixture was poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (silica gel, 50% to 100% EtOAc in hexane) to give 3,4,5-trifluoro-N-{cis-4-[(5-methyl-4-nitro-1-oxidopyridin-2-yl)amino]cyclohexyl}benzamide (1.97 g) as a yellow solid.

¹H NMR (300 MHz, DMSO-d₆, δ): 1.62-1.95 (m, 8H), 2.35 (s, 3H), 3.78-4.05 (m, 2H), 6.96 (d, J=8.9 Hz, 1H), 7.51 (s, 1H), 7.77-7.88 (m, 2H), 8.30-8.38 (m, 1H), 8.43 (s, 1H); ESI MS m/z 425 (M⁺+1, 65%), 447 (M⁺+23, 100%).

### Step C: Synthesis of N-{cis-4-[(4-amino-5-methylpyridin-2-yl)amino]cyclohexyl}-3,4,5-trifluorobenzamide

A mixture of 3,4,5-trifluoro-N-{cis-4-[(5-methyl-4-nitro-1-oxidopyridin-2-yl)amino]-cyclohexyl}benzamide (1.81 g), Fe (3.62 g), and AcOH (36 mL) was stirred at 100° C. for 15 min. The mixture was filtered through a pad of celite and the filtrate was poured into 1 M aqueous NaOH. The aqueous layer was extracted with EtOAc three times. The combined organic layer was dried over MgSO₄, filtered, concentrated under reduced pressure, and purified by medium-pressure liquid chromatography (NH-silica gel, 50% EtOAc in hexane to 10% MeOH in CHCl₃) to give N-{cis-4-[(4-amino-5-methylpyridin-2-yl)amino]cyclohexyl}-3,4,5-trifluorobenzamide (1.49 g) as a colorless solid.

¹H NMR (300 MHz, CDCl₃, δ): 1.69-1.88 (m, 8H), 2.00 (s, 3H), 3.55-3.64 (m, 1H), 4.05-4.15 (m, 1H), 4.51 (s, 2H), 5.69 (s, 1H), 7.32-7.41 (m, 2H), 7.51-7.62 (m, 2H); ESI MS m/z 379 (M⁺+1, 100%).

### Step D: Synthesis of 3,4,5-trifluoro-N-(cis-4-{[5-methyl-4-(methylamino)pyridin-2-yl]amino}cyclohexyl)benzamide hydrochloride

To a solution of N-{cis-4-[(4-amino-5-methylpyridin-2-yl)amino]cyclohexyl}-3,4,5-trifluorobenzamide (780 mg) in AcOH (20 mL) was added paraformaldehyde (69.0 mg). The mixture was stirred at ambient temperature for 1.5 h and NaBH₃CN (142 mg) was added. The mixture was stirred at ambient temperature for 10 h. To the mixture were added paraformaldehyde (69.0 mg) and NaBH₃CN (142 mg). The mixture was stirred at ambient temperature for 5 h and poured into 1 M aqueous HCl. The aqueous layer was extracted with CHCl₃ three times. The combined organic layer was dried over MgSO₄, filtered, concentrated under reduced pressure, and purified by medium-pressure liquid chromatography (silica gel, 5% to 15% MeOH in CHCl₃) to give a colorless solid. To a solution of the above solid in EtOAc (5 mL) was added 4 M hydrogen chloride in EtOAc (10 mL). The mixture was stirred at ambient temperature for 1 h and concentrated under reduced pressure. A suspension of the residue in Et₂O (20 mL) was stirred at ambient temperature for 4 h. The precipitate was collected by filtration, washed with Et₂O, and dried at 80° C. under reduced pressure to give 3,4,5-trifluoro-N-(cis-4-{[5-methyl-4-(methylamino)pyridin-2-yl]amino}cyclohexyl)benzamide hydrochloride (71.0 mg) as a white solid.

¹H NMR (300 MHz, CDCl₃, δ): 1.63-2.14 (m, 11H), 3.00 (d, J=4.2 Hz, 3H), 3.71-3.83 (m, 1H), 4.02-4.19 (m, 1H), 5.00 (brs, 1H), 5.48 (s, 1H), 7.08-7.24 (m, 2H), 7.51-7.64 (m, 2H), 8.16-8.25 (m, 1H), 12.84 (brs, 1H); ESI MS m/z 393 [M (free)⁺+1, 100%].

### Example 17

### N-{cis-4-[(4-Amino-5-methylpyridin-2-yl)amino]cyclohexyl}-3-chloro-4-fluorobenzamide hydrochloride

To a solution of N-{cis-4-[(4-amino-5-methylpyridin-2-yl)amino]cyclohexyl}-3-chloro-4-fluorobenzamide obtained in step B of example 15 (464 mg) in EtOAc (5 mL) was added 4 M hydrogen chloride in EtOAc (10 mL). The mixture was stirred at ambient temperature for 1 h and concentrated under reduced pressure. A suspension of the residue in Et₂O (20 mL) was stirred at ambient temperature for 4 h. The precipitate was collected by filtration, washed with Et₂O, and dried at 80° C. under reduced pressure to give N-{cis-4-[(4-amino-5-methylpyridin-2-yl)amino]cyclohexyl}-3-chloro-4-fluorobenzamide hydrochloride (419 mg) as a white solid.

¹H NMR (300 MHz, DMSO-d₆, δ): 1.54-1.88 (m, 8H), 1.94 (s, 3H), 3.49-3.63 (m, 1H), 3.80-3.97 (m, 1H) 5.91 (s, 1H), 6.90-7.29 (m, 2H), 7.40-7.57 (m, 3H), 7.87-7.94 (m, 1H), 8.11 (dd, J=7.3, 2.2 Hz, 1H), 8.40 (d, J=6.5 Hz, 1H), 12.01 (brs, 1H); ESI MS m/z 377 [M (free)⁺+1, 100%].

### Example 18

### N-{cis-4-[(4-Amino-5-methylpyridin-2-yl)amino]cyclohexyl}-3,4,5-trifluorobenzamide hydrochloride

To a solution of N-{cis-4-[(4-amino-5-methylpyridin-2-yl)amino]cyclohexyl}-3,4,5-trifluorobenzamide obtained in step C of example 16 (234 mg) in EtOAc (5 mL) was added 4 M hydrogen chloride in EtOAc (10 mL). The mixture was stirred at ambient temperature for 1 h and concentrated under reduced pressure. A suspension of the residue in Et₂O (20 mL) was stirred at ambient temperature for 4 h. The precipitate was collected by filtration, washed with Et₂O, and dried at 80° C. under reduced pressure to give N-{cis-4-[(4-amino-5-methylpyridin-2-yl)amino]cyclohexyl}-3,4,5-trifluorobenzamide hydrochloride (171 mg) as a white solid.

¹H NMR (300 MHz, DMSO-d₆, δ): 1.57-1.88 (m, 8H), 1.94 (s, 3H), 3.47-3.61 (m, 1H), 3.81-3.94 (m, 1H), 5.90 (s, 1H), 6.88-7.33 (m, 2H), 7.40-7.48 (m, 2H), 7.80-7.91 (m, 2H), 8.44 (d, J=6.5 Hz, 1H), 11.95 (brs, 1H); ESI MS m/z 379 [M (free)⁺+1, 100%].

### Example 19

### 3-Chloro-N-[cis-4-({4-[ethyl(methyl)amino]-5-methylpyridin-2-yl}amino)cyclohexyl]-4-fluorobenzamide hydrochloride

### Step A: Synthesis of N²-[cis-4-(benzylamino)cyclohexyl]-N⁴-ethyl-N⁴,5-dimethylpyridine-2,4-diamine

A mixture of cis-N-benzyl-N′-(4-chloro-5-methyl-1-oxidopyridin-2-yl)cyclohexane-1,4-diamine obtained in step D of example 4 (870 mg), N-ethylmethylamine (647 μL), and BuOH (0.9 mL) was heated in a microwave synthesizer at 220° C. for 20 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 20% to 50% EtOAc in hexane) to give N²-[cis-4-(benzylamino)cyclohexyl]-N⁴-ethyl-N⁴,5-dimethylpyridine-2,4-diamine (260 mg) as a pale brown oil.

¹H NMR (300 MHz, CDCl₃, δ): 1.13 (t, J=7.2 Hz, 3H), 1.45-1.92 (m, 8H), 2.10 (s, 3H), 2.63-2.75 (m, 4H), 3.03 (q, J=7.2 Hz, 2H), 3.74-3.85 (m, 3H), 4.30-4.39 (m, 1H), 5.83 (s, 1H), 7.17-7.40 (m, 5H), 7.72 (s, 1H); ESI MS m/z 353 (M⁺+1, 100%).

### Step B: Synthesis of N²-(cis-4-aminocyclohexyl)-N⁴-ethyl-N⁴,5-dimethylpyridine-2,4-diamine

A mixture of N²-[cis-4-(benzylamino)cyclohexyl]-N⁴-ethyl-N⁴,5-dimethylpyridine-2,4-diamine (260 mg), cyclohexene (1.52 mL), 10% palladium carbon (180 mg), and 2-propanol (5.00 mL) was stirred at reflux for 60 h. The mixture was filtered through a pad of celite, concentrated under reduced pressure, and purified by medium-pressure liquid chromatography (NH-silica gel, 2% MeOH in CHCl₃) to give N²-(cis-4-aminocyclohexyl)-N⁴-ethyl-N⁴,5-dimethylpyridine-2,4-diamine (78.0 mg) as a pale brown oil.

¹H NMR (300 MHz, CDCl₃, δ): 1.14 (t, J=7.2 Hz, 3H), 1.33-1.87 (m, 8H), 2.10 (s, 3H), 2.73 (s, 3H), 2.80-2.92 (m, 1H), 3.04 (q, J=7.2 Hz, 2H), 3.70-3.82 (m, 1H), 4.34-4.42 (m, 1H), 5.84 (s, 1H), 7.71 (s, 1H); CI MS m/z 263 (M⁺+1, 100%).

### Step C: Synthesis of 3-chloro-N-[cis-4-({4-[ethyl(methyl)amino]-5-methylpyridin-2-yl}amino)cyclohexyl]-4-fluorobenzamide hydrochloride

To a solution of N²-(cis-4-aminocyclohexyl)-N⁴-ethyl-N⁴,5-dimethylpyridine-2,4-diamine (78.0 mg) in DMF (1 mL) were added 3-chloro-4-fluorobenzoic acid (62.0 mg), Et₃N (99.0 μL), HOBt-H₂O (68.0 mg), and EDC-HCl (68.0 mg). The mixture was stirred at ambient temperature for 12 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-N-[cis-4-({4-[ethyl(methyl)amino]-5-methylpyridin-2-yl}amino)cyclohexyl]-4-fluorobenzamide hydrochloride (18.0 mg) as a white solid.

¹H NMR (300 MHz, CDCl₃, δ): 1.27 (t, J=7.2 Hz, 3H), 1.65-2.09 (m, 8H), 2.19 (s, 3H), 2.99 (s, 3H), 3.37 (q, J=7.2 Hz, 2H), 3.67-3.78 (m, 1H), 4.03-4.21 (m, 1H), 5.69 (s, 1H), 6.76-6.88 (m, 1H), 7.11-7.29 (m, 2H), 7.68-7.79 (m, 1H), 7.96 (dd, J=7.0, 1.9 Hz, 1H), 8.20-8.31 (m, 1H), 13.24 (brs, 1H); ESI MS m/z 419 [M (free)⁺+1, 100%].

### Example 20

### 3-Chloro-4-fluoro-N-{cis-4-[(5-methyl-4-pyrrolidin-1-ylpyridin-2-yl)amino]cyclohexyl}-benzamide hydrochloride

### Step A: Synthesis of cis-N-benzyl-N′-(5-methyl-4-pyrrolidin-1-ylpyridin-2-yl)cyclohexane-1,4-diamine

A mixture of cis-N-benzyl-N′-(4-chloro-5-methyl-1-oxidopyridin-2-yl)cyclohexane-1,4-diamine obtained in step D of example 4 (900 mg), pyrrolidine (222 mg), and BuOH (0.9 mL) was heated in a microwave synthesizer at 220° C. for 20 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 10% to 50% EtOAc in hexane) to give cis-N-benzyl-N′-(5-methyl-4-pyrrolidin-1-ylpyridin-2-yl)cyclohexane-1,4-diamine (269 mg) as a pale brown oil.

¹H NMR (300 MHz, CDCl₃, δ): 1.47-1.85 (m, 8H), 1.88-1.98 (m, 4H), 2.21 (s, 3H), 2.62-2.73 (m, 1H), 3.30-3.44 (m, 4H), 3.71-3.83 (m, 3H), 4.24-4.34 (m, 1H), 5.57 (s, 1H), 7.21-7.37 (m, 5H), 7.59 (s, 1H); ESI MS m/z 365 (M⁺+1, 100%).

### Step B: Synthesis of 3-chloro-4-fluoro-N-{cis-4-[(5-methyl-4-pyrrolidin-1-ylpyridin-2-yl)amino]cyclohexyl}benzamide hydrochloride

A mixture of cis-N-benzyl-N′-(5-methyl-4-pyrrolidin-1-ylpyridin-2-yl)cyclohexane-1,4-diamine (265 mg), 10% palladium carbon (53.0 mg), and MeOH (2.6 mL) was stirred at 50° C. under hydrogen atmosphere for 24 h. The mixture was filtered through a pad of celite, concentrated under reduced pressure, and purified by medium-pressure liquid chromatography (NH-silica gel, 20% EtOAc in hexane to 5% MeOH in CHCl₃) to give a pale brown oil (102 mg). To a solution of the above oil (95.0 mg) in DMF (0.5 mL) were added 3-chloro-4-fluorobenzoic acid (72.0 mg), Et₃N (116 μL), HOBt-H₂O (80.0 mg), and EDC-HCl (80.0 mg). The mixture was stirred at ambient temperature for 12 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane and silica gel, 3% to 9% MeOH in CHCl₃). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-{cis-4-[(5-methyl-4-pyrrolidin-1-ylpyridin-2-yl)amino]cyclohexyl}benzamide hydrochloride (15.0 mg) as a white solid.

¹H NMR (300 MHz, CDCl₃, δ): 1.62-2.25 (m, 12H), 2.30 (s, 3H), 3.48-3.80 (m, 5H), 4.02-4.23 (m, 1H), 5.43 (s, 1H), 7.08-7.24 (m, 3H), 7.72-7.85 (m, 1H), 7.87-8.08 (m, 2H), 12.72 (brs, 1H); ESI MS m/z 431 [M (free)⁺+1, 100%].

### Example 21

### 3-Chloro-4-fluoro-N-{cis-4-[(5-methyl-4-morpholin-4-ylpyridin-2-yl)amino]cyclohexyl}-benzamide hydrochloride

### Step A: Synthesis of cis-N-benzyl-N′-(5-methyl-4-morpholin-4-ylpyridin-2-yl)cyclohexane-1,4-diamine

A mixture of cis-N-benzyl-N′-(4-chloro-5-methyl-1-oxidopyridin-2-yl)cyclohexane-1,4-diamine obtained in step D of example 4 (900 mg), morpholine (272 mg), and BuOH (0.9 mL) was heated in a microwave synthesizer at 220° C. for 20 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 14% to 66% EtOAc in hexane) to give cis-N-benzyl-N′-(5-methyl-4-morpholin-4-ylpyridin-2-yl)cyclohexane-1,4-diamine (322 mg) as a brown oil.

¹H NMR (300 MHz, CDCl₃, δ): 1.45-1.90 (m, 8H), 2.10 (s, 3H), 2.64-2.76 (m, 1H), 2.89-3.05 (m, 4H), 3.61-3.94 (m, 7H), 4.41 (d, J=7.9 Hz, 1H), 5.86 (s, 1H), 7.19-7.40 (m, 5H), 7.78 (s, 1H); ESI MS m/z 381 (M⁺+1, 100%).

### Step B: Synthesis of cis-N-(5-methyl-4-morpholin-4-ylpyridin-2-yl)cyclohexane-1,4-diamine

A mixture of cis-N-benzyl-N′-(5-methyl-4-morpholin-4-ylpyridin-2-yl)cyclohexane-1,4-diamine (315 mg), 10% palladium carbon (63.0 mg), and MeOH (3.2 mL) was stirred at 50° C. under hydrogen atmosphere for 24 h. The mixture was filtered through a pad of celite, concentrated under reduced pressure, and purified by medium-pressure liquid chromatography (NH-silica gel, 33% EtOAc in hexane to 3% MeOH in CHCl₃) to give cis-N-(5-methyl-4-morpholin-4-ylpyridin-2-yl)cyclohexane-1,4-diamine (193 mg) as a brown oil.

¹H NMR (300 MHz, CDCl₃, δ): 1.34-1.90 (m, 8H), 2.11 (s, 3H), 2.81-3.06 (m, 5H), 3.70-3.92 (m, 5H), 4.36-4.49 (m, 1H), 5.87 (s, 1H), 7.78 (s, 1H); ESI MS m/z 291 (M⁺+1, 100%).

### Step C: Synthesis of 3-chloro-4-fluoro-N-{cis-4-[(5-methyl-4-morpholin-4-ylpyridin-2-yl)amino]cyclohexyl}benzamide hydrochloride

To a solution of cis-N-(5-methyl-4-morpholin-4-ylpyridin-2-yl)cyclohexane-1,4-diamine (185 mg) in DMF (2 mL) were added 3-chloro-4-fluorobenzoic acid (132 mg), Et₃N (210 μL), HOBt-H₂O (144 mg), and EDC-HCl (145 mg). The mixture was stirred at ambient temperature for 12 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane and silica gel, 3% to 9% MeOH in CHCl₃). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-{cis-4-[(5-methyl-4-morpholin-4-ylpyridin-2-yl)amino]cyclohexyl}benzamide hydrochloride (166 mg) as a white solid.

¹H NMR (200 MHz, CDCl₃, δ): 1.57-1.99 (m, 8H), 2.12 (s, 3H), 2.90-3.04 (m, 4H), 3.72-3.92 (m, 5H), 4.04-4.22 (m, 1H), 4.34-4.48 (m, 1H), 5.89 (s, 1H), 5.96-6.11 (m, 1H), 7.14-7.25 (m, 1H), 7.59-7.70 (m, 1H), 7.76-7.87 (m, 2H); ESI MS m/z 447 [M (free)⁺+1, 100%].

### Example 22

### 3-Chloro-4-fluoro-N-(cis-4-{[4-(1H-imidazol-1-yl)-5-methylpyridin-2-yl]amino}cyclohexyl)-benzamide dihydrochloride

### Step A: Synthesis of cis-N-[4-(1H-imidazol-1-yl)-5-methylpyridin-2-yl]cyclohexane-1,4-diamine

A mixture of cis-N-benzyl-N′-(4-chloro-5-methyl-1-oxidopyridin-2-yl)cyclohexane-1,4-diamine obtained in step D of example 4 (900 mg), 1H-imidazol (212 mg), and BuOH (0.9 mL) was heated in a microwave synthesizer at 220° C. for 35 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 20% to 100% EtOAc in hexane) to give a brown oil (137 mg). A mixture of the above oil (130 mg), 10% palladium carbon (26.0 mg), and MeOH (2.5 mL) was stirred at 50° C. under hydrogen atmosphere for 70 h. The mixture was filtered through a pad of celite, concentrated under reduced pressure, and purified by medium-pressure liquid chromatography (NH-silica gel, 50% EtOAc in hexane to 9% MeOH in CHCl₃) to give cis-N-[4-(1H-imidazol-1-yl)-5-methylpyridin-2-yl]cyclohexane-1,4-diamine (88.0 mg) as a pale brown oil.

¹H NMR (300 MHz, CDCl₃, δ): 1.34-1.90 (m, 8H), 2.09 (s, 3H), 2.85-2.97 (m, 1H), 3.66-3.81 (m, 1H), 4.72 (d, J=7.8 Hz, 1H), 6.22 (s, 1H), 7.09-7.11 (m, 1H), 7.20-7.23 (m, 1H), 7.65 (s, 1H), 8.04 (s, 1H); ESI MS m/z 272 (M⁺+1, 100%).

### Step B: Synthesis of 3-chloro-4-fluoro-N-(cis-4-{[4-(1H-imidazol-1-yl)-5-methylpyridin-2-yl]amino}cyclohexyl)benzamide dihydrochloride

To a solution of cis-N-[4-(1H-imidazol-1-yl)-5-methylpyridin-2-yl]cyclohexane-1,4-diamine (80.0 mg) in DMF (2 mL) were added 3-chloro-4-fluorobenzoic acid (62.0 mg), Et₃N (97.0 μL), HOBt-H₂O (67.0 mg), and EDC-HCl (67.0 mg). The mixture was stirred at ambient temperature for 60 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 25% to 100% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-(cis-4-{[4-(1H-imidazol-1-yl)-5-methylpyridin-2-yl]amino}cyclohexyl)benzamide dihydrochloride (70.0 mg) as a white solid.

¹H NMR (200 MHz, DMSO-d₆, δ): 1.63-1.94 (m, 8H), 2.08 (s, 3H), 3.81-4.00 (m, 2H), 7.08 (brs, 1H), 7.52 (t, J=9.0 Hz, 1H), 7.80-7.96 (m, 2H), 7.98-8.15 (m, 3H), 8.35-8.44 (m, 1H), 9.30 (brs, 1H); ESI MS m/z 428 [M (free)⁺+1, 100%].

### Example 23

### N²-{cis-4-[(3-Chloro-4-fluorobenzyl)amino]cyclohexyl}-N⁴,N⁴,5-trimethylpyridine-2,4-diamine dihydrochloride

To a solution of N²-(cis-4-aminocyclohexyl)-N⁴,N⁴,5-trimethylpyridine-2,4-diamine obtained in step F of example 4 (160 mg) in CHCl₃ (2 mL) were added AcOH (38.0 mg) and 3-chloro-4-fluorobenzaldehyde (101 mg). The mixture was stirred at ambient temperature for 1 h and NaBH(OAc)₃ (163 mg) was added. The mixture was stirred at ambient temperature for 14 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 50% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give N²-{cis-4-[(3-chloro-4-fluorobenzyl)amino]cyclohexyl}-N⁴,N⁴, 5-trimethylpyridine-2,4-diamine dihydrochloride (161 mg) as a white solid.

¹H NMR (300 MHz, DMSO-d₆, δ): 1.58-2.02 (m, 8H), 2.19 (s, 3H), 3.00 (s, 6H), 3.07-3.21 (m, 1H), 3.78-3.88 (m, 1H), 4.14-4.25 (m, 2H), 6.12 (brs, 1H), 7.46-7.55 (m, 2H), 7.64-7.71 (m, 1H), 7.87-7.99 (m, 2H), 9.36-9.60 (m, 2H), 12.51-12.79 (m, 1H); ESI MS m/z 391 [M (free)⁺+1, 100%].

### Example 24

### 3-Chloro-N-(cis-4-{[4-(dimethylamino)-5-methylpyridin-2-yl]amino}cyclohexyl)-4-fluorobenzenesulfonamide hydrochloride

To a solution of N²-(cis-4-aminocyclohexyl)-N⁴,N⁴,5-trimethylpyridine-2,4-diamine obtained in step F of example 4 (200 mg) in CHCl₃ (2 mL) were added Et₃N (238 μL) and 3-chloro-4-fluorobenzenesulfonyl chloride (221 mg) in CHCl₃ (1 mL). The mixture was stirred at ambient temperature for 12 h and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 50% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-N-(cis-4-{[4-(dimethylamino)-5-methylpyridin-2-yl]amino}cyclohexyl)-4-fluorobenzenesulfonamide hydrochloride (188 mg) as a white solid.

¹H NMR (300 MHz, CDCl₃, δ): 1.55-1.96 (m, 8H), 2.18 (s, 3H), 3.03 (s, 6H), 3.24-3.40 (m, 1H), 3.52-3.63 (m, 1H), 5.61 (s, 1H), 6.24-6.34 (m, 1H), 7.20-7.34 (m, 2H), 7.89-7.97 (m, 1H), 8.01-8.17 (m, 2H); ESI MS m/z 441 [M (free)⁺+1, 100%].

### Example 25

### N-(3-Chloro-4-fluorophenyl)-N′-(cis-4-{[4-(dimethylamino)-5-methylpyridin-2-yl]amino}cyclohexyl)urea hydrochloride

To a solution of N²-(cis-4-aminocyclohexyl)-N⁴,N⁴,5-trimethylpyridine-2,4-diamine obtained in step F of example 4 (200 mg) in DMSO (2 mL) was added 2-chloro-1-fluoro-4-isocyanatobenzene (153 mg) in DMSO (1 mL). The mixture was stirred at ambient temperature for 12 h and poured into water. The precipitate was collected by filtration, washed with water, and purified by medium-pressure liquid chromatography (NH-silica gel, 20% to 50% EtOAc in hexane). To a solution of the above purified material in EtOAc (10 mL) was added 4 M hydrogen chloride in EtOAc (0.4 mL). The mixture was stirred at ambient temperature for 1 h and concentrated under reduced pressure. A suspension of the residue in Et₂O (10 mL) was stirred at ambient temperature for 3 h. The precipitate was collected by filtration, washed with Et₂O, and dried at 80° C. under reduced pressure to give N-(3-chloro-4-fluorophenyl)-N′-(cis-4-{[4-(dimethylamino)-5-methylpyridin-2-yl]amino}cyclohexyl)urea hydrochloride (138 mg) as a white solid.

¹H NMR (300 MHz, CDCl₃, δ): 1.66-1.95 (m, 8H), 2.15 (s, 3H), 3.03 (s, 6H), 3.43-3.56 (m, 1H), 3.82-3.96 (m, 1H), 5.64 (s, 1H), 6.54-6.64 (m, 1H), 6.93 (t, J=8.9 Hz, 1H), 7.24 (brs, 1H), 7.29-7.37 (m, 1H), 7.43-7.51 (m, 1H), 7.59 (dd, J=6.6, 2.6 Hz, 1H), 8.76 (s, 1H), 12.57-12.72 (m, 1H); ESI MS m/z 420 [M (free)⁺+1, 100%].

### Example 26

### N-(3-Chloro-4-fluorophenyl)-N′-(cis-4-{[4-(dimethylamino)-5-methylpyridin-2-yl]amino}-cyclohexyl)thiourea hydrochloride

To a solution of N²-(cis-4-aminocyclohexyl)-N⁴,N⁴,5-trimethylpyridine-2,4-diamine obtained in step F of example 4 (200 mg) in DMSO (2 mL) was added 2-chloro-1-fluoro-4-isothiocyanatobenzene (167 mg) in DMSO (1 mL). The mixture was stirred at ambient temperature for 12 h and poured into water. The precipitate was collected by filtration, washed with water, and purified by medium-pressure liquid chromatography (NH-silica gel, 20% to 50% EtOAc in hexane). To a solution of the above purified material in EtOAc (10 mL) was added 4 M hydrogen chloride in EtOAc (0.4 mL). The mixture was stirred at ambient temperature for 1 h and concentrated under reduced pressure. A suspension of the residue in Et₂O (10 mL) was stirred at ambient temperature for 3 h. The precipitate was collected by filtration, washed with Et₂O, and dried at 80° C. under reduced pressure to give N-(3-chloro-4-fluorophenyl)-N′-(cis-4-{[4-(dimethylamino)-5-methylpyridin-2-yl]amino}cyclohexyl)thiourea hydrochloride (157 mg) as a white solid.

¹H NMR (300 MHz, CDCl₃, δ): 1.68-2.07 (m, 8H), 2.15 (s, 3H), 3.05 (s, 6H), 3.47-3.59 (m, 1H), 4.47-4.58 (m, 1H), 5.60 (s, 1H), 7.03 (t, J=8.8 Hz, 1H), 7.10-7.16 (m, 1H), 7.42-7.50 (m, 1H), 7.54-7.63 (m, 2H), 7.89-7.99 (m, 1H), 9.40 (s, 1H), 12.46-12.58 (m, 1H); ESI MS m/z 436 [M (free)⁺+1, 100%].

### Example 27

### 4-Bromophenyl (cis-4-{[4-(dimethylamino)-5-methylpyridin-2-yl]amino}cyclohexyl)carbamate

To a solution of N²-(cis-4-aminocyclohexyl)-N⁴,N⁴,5-trimethylpyridine-2,4-diamine obtained in step F of example 4 (200 mg) in CHCl₃ (2 mL) were added Et₃N (238 μL) and 4-bromophenyl chloridocarbonate (210 mg) in CHCl₃ (1 mL). The mixture was stirred at ambient temperature for 12 h and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (silica gel, 3% to 9% MeOH in CHCl₃) to give 4-bromophenyl (cis-4-{[4-(dimethylamino)-5-methylpyridin-2-yl]amino}-cyclohexyl)carbamate (105 mg) as a colorless oil.

¹H NMR (300 MHz, CDCl₃, δ): 1.60-1.92 (m, 8H), 2.14 (s, 3H), 2.78 (s, 6H), 3.68-3.82 (m, 2H), 4.33-4.42 (m, 1H), 5.03-5.10 (m, 1H), 5.83 (s, 1H), 7.00-7.06 (m, 2H), 7.43-7.49 (m, 2H), 7.71 (s, 1H); ESI MS m/z 447 (M⁺+1, 98%), 449 (M⁺+3, 100%)

### Example 28

### 3-Chloro-N-(cis-4-{[4-(dimethylamino)-6-methylpyridin-2-yl]amino}cyclohexyl)-4-fluorobenzamide hydrochloride

### Step A: Synthesis of 6-methylpyridine-2,4-diol

A suspension of 3-acetyl-6-methyl-2H-pyran-2,4(3H)-dione (500 g) in concentrated H₂SO₄ (800 mL) was stirred at 130° C. for 2 h. The mixture was poured into ice-water. The precipitate was collected by filtration, washed with hexane, and dried at 80° C. A suspension of the above solid in 28% aqueous NH₃ was stirred at 100° C. for 7 h. To the mixture was added water (1 L) and the mixture was neutralizing with concentrated HCl. The precipitate was collected by filtration, washed with water and acetone, and dried at 80° C. to give 6-methylpyridine-2,4-diol (135 g) as a pale brown solid.

¹H NMR (300 MHz, DMSO-d₆, δ): 2.08 (s, 3H), 5.34-5.37 (m, 1H), 5.60-5.63 (m, 1H), 10.30-10.61 (m, 1H), 10.92-11.15 (m, 1H); ESI MS m/z 126 (M⁺+1, 100%).

### Step B: Synthesis of 2,4-dichloro-6-methylpyridine

To a suspension of 6-methylpyridine-2,4-diol (135 g) in POCl₃ (211 mL) was added N,N-dimethylaniline (150 g). The mixture was stirred at reflux for 30 min and poured into ice-water. The aqueous layer was extracted with CHCl₃ three times. The combined organic layer was dried over MgSO₄, filtered, and concentrated under reduced pressure to give 2,4-dichloro-6-methylpyridine (135 g) as a pale yellow oil.

¹H NMR (300 MHz, CDCl₃, δ): 2.53 (s, 3H), 7.09-7.11 (m, 1H), 7.17-7.20 (m, 1H); CI MS m/z 162 (M⁺+1, 100%).

### Step C: Synthesis of cis-N-benzyl-N′-(4-chloro-6-methyl-1-oxidopyridin-2-yl) cyclohexane-1,4-diamine

To a solution of 2,4-dichloro-6-methylpyridine (38.8 g) in CHCl₃ (388 mL) was added a solution of mCPBA (63.6 g) in CHCl₃ (318 mL) under 10° C. The mixture was stirred at 4° C. for 1.5 h, at ambient temperature for 2 h, and at 50° C. for 15 h and quenched with aqueous saturated Na₂S₂O₃. The aqueous layer was extracted with CHCl₃ three times. The combined organic layer was washed with 1 M aqueous NaOH (500 mL) and aqueous saturated NaCl (500 mL), dried over MgSO₄, filtered, concentrated under reduced pressure, and purified by medium-pressure liquid chromatography (silica gel, 20% to 100% EtOAc in hexane) to give a white solid (27.0 g). A mixture of the above solid (3.00 g), cis-N-benzylcyclohexane-1,4-diamine obtained in step C of example 4 (4.13 g), and BuOH (6 mL) was stirred at reflux for 6 h and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (silica gel, CHCl₃ to 10% MeOH in CHCl₃) to give cis-N-benzyl-N′-(4-chloro-6-methyl-1-oxidopyridin-2-yl)cyclohexane-1,4-diamine (4.89 g) as a brown oil.

¹H NMR (300 MHz, CDCl₃, δ): 1.53-2.00 (m, 8H), 2.49 (s, 3H), 2.69-2.81 (m, 1H), 3.41-3.54 (m, 1H), 3.80 (s, 2H), 6.42-6.45 (m, 1H), 6.49-6.51 (m, 1H), 7.14-7.38 (m, 5H); ESI MS m/z 346 (M⁺+1, 100%).

### Step D: Synthesis of N²-[cis-4-(benzylamino)cyclohexyl]-N⁴,N⁴,6-trimethylpyridine-2,4-diamine 1-oxide

A mixture of cis-N-benzyl-N′-(4-chloro-6-methyl-1-oxidopyridin-2-yl)cyclohexane-1,4-diamine (3.80 g), 50% aqueous Me₂NH (5.94 g), and BuOH (3 mL) was heated in a microwave synthesizer at 160° C. for 1.5 hr. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% EtOAc in hexane to 9% MeOH in CHCl₃) to give N²-[cis-4-(benzylamino)cyclohexyl]-N⁴,N⁴,6-trimetxylpyridine-2,4-diamine 1-oxide (2.24 g) as a brown oil.

¹H NMR (300 MHz, CDCl₃, δ): 1.55-2.00 (m, 8H), 2.47 (s, 3H), 2.64-2.77 (m, 1H), 2.96 (s, 6H), 3.42-3.53 (m, 1H), 3.80 (s, 2H), 5.51-5.55 (m, 1H), 5.81-5.86 (m, 1H), 7.03-7.14 (m, 1H), 7.18-7.37 (m, 5H); ESI MS m/z 355 (M⁺+1, 100%).

### Step E: Synthesis of N²-(cis-4-aminocyclohexyl)-N⁴,N⁴,6-trimethylpyridine-2,4-diamine

A mixture of N²-[cis-4-(benzylamino)cyclohexyl]-N⁴,N⁴,6-trimethylpyridine-2,4-diamine 1-oxide (2.06 g), 10% palladium carbon (206 mg), and MeOH (27 mL) was stirred at 50° C. under hydrogen atmosphere for 7 days. The mixture was filtered through a pad of celite and concentrated under reduced pressure to give N²-(cis-4-aminocyclohexyl)-N⁴,N⁴,6-trimethylpyridine-2,4-diamine (1.85 g).

¹H NMR (300 MHz, DMSO-d₆, δ): 1.57-1.92 (m, 8H), 2.30 (s, 3H), 3.08 (s, 6H), 3.74-3.96 (m, 2H), 5.62 (brs, 1H), 6.25 (brs, 1H), 7.55-7.60 (m, 1H), 8.26 (brs, 2H); ESI MS m/z 249 (M⁺+1, 100%).

### Step F: Synthesis of 3-chloro-N-(cis-4-{[4-(dimethylamino)-6-methylpyridin-2-yl]amino}cyclohexyl)-4-fluorobenzamide hydrochloride

To a solution of N²-(cis-4-aminocyclohexyl)-N⁴,N⁴,6-trimethylpyridine-2,4-diamine (400 mg) in DMF (4 mL) were added 3-chloro-4-fluorobenzoic acid (309 mg), Et₃N (540 μL), HOBt-H₂O (296 mg), and EDC-HCl (340 mg). The mixture was stirred at ambient temperature for 15 h and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane and silica gel, 3% to 10% MeOH in CHCl₃). To a solution of the above purified material in EtOAc (5 mL) was added 4 M hydrogen chloride in EtOAc (10 mL). The mixture was stirred at ambient temperature for 1 h and concentrated under reduced pressure. A suspension of the residue in Et₂O (20 mL) was stirred at ambient temperature for 3 h. The precipitate was collected by filtration, washed with Et₂O, and dried at 80° C. under reduced pressure to give 3-chloro-N-(cis-4-{[4-(dimethylamino)-6-methylpyridin-2-yl]amino}cyclohexyl)-4-fluorobenzamide hydrochloride (128 mg) as a white solid.

¹H NMR (300 MHz, CDCl₃, δ): 1.63-2.04 (m, 8H), 2.41 (s, 3H), 3.11 (s, 6H), 3.62-3.80 (m, 1H), 4.02-4.23 (m, 1H), 5.32 (brs, 1H), 5.84 (brs, 1H), 6.86-6.98 (m, 1H), 7.17 (t, J=8.6 Hz, 1H), 7.70-7.80 (m, 1H), 7.92-8.13 (m, 2H), 13.04 (brs, 1H); ESI MS m/z 405 [M (free)⁺+1, 100%].

### Example 29

### N-(cis-4-{[4-(Dimethylamino)-6-methylpyridin-2-yl]amino}cyclohexyl)-3,4,5-trifluorobenzamide hydrochloride

Using the procedure for the step F of example 28, the title compound was obtained.

¹H NMR (300 MHz, CDCl₃, δ): 1.61-2.07 (m, 8H), 2.41 (s, 3H), 3.11 (s, 6H), 3.66-3.77 (m, 1H), 4.04-4.18 (m, 1H), 5.32 (brs, 1H), 5.85 (brs, 1H), 6.95-7.05 (m, 1H), 7.51-7.62 (m, 2H), 8.00-8.08 (m, 1H), 13.01 (brs, 1H); ESI MS m/z 407 [M (free)⁺+1, 100%].

### Example 30

### 3-Chloro-N-{cis-4-[(6-chloropyridin-2-yl)amino]cyclohexyl}-4-fluorobenzamide hydrochloride

Using the procedure for the step B of example 1, the title compound was obtained.

¹H NMR (300 MHz, CDCl₃, δ): 1.71-2.01 (m, 8H), 3.75-3.83 (m, 1H), 4.06-4.19 (m, 1H), 6.28-6.37 (m, 1H), 6.46 (d, J=8.6 Hz, 1H), 6.64 (d, J=7.2 Hz, 1H), 7.15-7.24 (m, 1H), 7.53 (dd, J=8.6, 7.5 Hz, 1H), 7.64-7.72 (m, 1H), 7.89 (dd, J=7.0, 2.9 Hz, 1H); ESI MS m/z 382 [M (free)⁺+1, 100%].

### Example 31

### 3-Chloro-N-[cis-4-({[4-(dimethylamino)-5-methylpyridin-2-yl]amino}methyl)cyclohexyl]-4-fluorobenzamide hydrochloride

### Step A: Synthesis of benzyl [(cis-4-aminocyclohexyl)methyl]carbamate

To a solution of benzyl ({cis-4-[(tert-butoxycarbonyl)amino]cyclohexyl}methyl)carbamate (60 g) in EtOAc (300 mL) was added 4 M hydrogen chloride in EtOAc (124 mL), and stirred at ambient temperature for 16 h. The mixture was added 4 M hydrogen chloride in EtOAc (124 mL) again, stirred at ambient temperature for 4.5 h, and concentrated under reduced pressure. The residue was diluted with CHCl₃ and cooled on an ice-bath. To the solution was added aqueous 1 M NaOH, and vigorously stirred at ambient temperature. The aqueous layer was extracted with CHCl₃ three times. The combined organic layer was dried over MgSO₄, filtrated, concentrated under reduced pressure to give benzyl [(cis-4-aminocyclohexyl)methyl]carbamate (51.5 g) as a yellow oil.

¹H NMR (300 MHz, CDCl₃, δ): 1.19-1.67 (m, 9H), 2.95-3.04 (m, 1H), 3.13 (t, J=6.6 Hz, 2H), 4.79 (brs, 1H), 5.10 (s, 2H), 5.15 (brs, 1H), 7.28-7.42 (m, 5H); ESI MS m/z 263 (M⁺+1, 100%).

### Step B: Synthesis of benzyl {[cis-4-(benzylamino)cyclohexyl]methyl}carbamate

A mixture of benzyl [(cis-4-aminocyclohexyl)methyl]carbamate (51.2 g), benzaldehyde (20.7 g), acetic acid (11.7 g), and CHCl₃ (300 mL) was stiffed at ambient temperature for 80 min. To the solution cooled on an ice-bath was added NaBH(OAc)₃ (52.3 g) and the mixture was stirred at ambient temperature for 14 h. After cooling on an ice-bath, aqueous 1 M NaOH (250 mL) was added to the reaction mixture. The aqueous layer was extracted with CHCl₃ three times. The combined organic layer was dried over MgSO₄, filtrated, concentrated under reduced pressure, and purified by medium-pressure liquid chromatography (silica gel, 9% MeOH in CHCl₃ to MeOH) to give benzyl {[cis-4-(benzylamino)cyclohexyl]methyl}carbamate (69.2 g) as a pale yellow oil.

¹H NMR (300 MHz, CDCl₃, δ): 1.43-1.85 (m, 8H), 2.86-2.96 (m, 1H), 3.14 (t, J=6.3 Hz, 2H), 3.94 (s, 2H), 5.09 (s, 2H), 5.11-5.18 (m, 1H), 5.49 (brs, 2H), 7.27-7.40 (m, 10H); ESI MS m/z 353 (M⁺+1, 100%).

### Step C: Synthesis of cis-4-(aminomethyl)-N-benzylcyclohexanamine

To a solution of benzyl {[cis-4-(benzylamino)cyclohexyl]methyl}carbamate (68.9 g) in MeOH (200 mL) was added a solution of KOH (19.3 g) in H₂O (30 mL). The mixture was stirred at ambient temperature for 66 h and at 100° C. for 3 h. After to the mixture was added H₂O (70 mL), the mixture was stirred at 100° C. for 4.5 h and at ambient temperature for 12 h. To the mixture was added KOH (19.3 g) in H₂O (10 mL) and the mixture was stirred at 100° C. for 55.5 h and at ambient temperature for 63 h. The reaction mixture was cooled on an ice-bath and acidified with conc. HCl (pH=1). The aqueous layer was extracted with CHCl₃ twice, cooled on an ice-bath, and alkalized with aqueous 50% NaOH. The aqueous layer was extracted with CHCl₃ three times. To the aqueous layer was added aqueous 50% NaOH and the aqueous layer was extracted with CHCl₃ three times. All of the combined organic layers were dried over MgSO₄, filtrated, and concentrated under reduced pressure to give cis-4-(aminomethyl)-N-benzylcyclohexanamine (21.9 g) as a pale yellow oil.

¹H NMR (300 MHz, CDCl₃, δ): 1.36-1.76 (m, 9H), 2.62 (d, J=5.0 Hz, 2H), 2.74-2.82 (m, 1H), 3.78 (s, 2H), 7.17-7.41 (m, 5H); ESI MS m/z 219 (M⁺+1, 100%).

### Step D: Synthesis of N-{[cis-4-(benzylamino)cyclohexyl]methyl}-4-chloro-5-methylpyridin-2-amine 1-oxide

A mixture of 2,4-dichloro-5-methylpyridine 1-oxide obtained in step B of example 4 (5.00 g), cis-4-(aminomethyl)-N-benzylcyclohexanamine (7.36 g), and BuOH (5 mL) was stirred at reflux for 9 h. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane) to give N-{[cis-4-(benzylamino)cyclohexyl]methyl}-4-chloro-5-methylpyridin-2-amine 1-oxide (4.19 g) as a brown solid.

¹H NMR (200 MHz, CDCl₃, δ): 1.42-1.79 (m, 9H), 2.19 (s, 3H), 2.74-2.87 (m, 1H), 3.08-3.20 (m, 2H), 3.77 (s, 2H), 6.57 (s, 1H), 6.62-6.74 (m, 1H), 7.19-7.37 (m, 5H), 7.97 (s, 1H); ESI MS m/z 360 (M⁺+1, 100%).

### Step E: Synthesis of N²-[(cis-4-benzylaminocyclohexyl)methyl]-N⁴,N⁴,5-trimethylpyridine-2,4-diamine 1-oxide

A mixture of N-{[cis-4-(benzylamino)cyclohexyl]methyl}-4-chloro-5-methylpyridin-2-amine 1-oxide (2.00 g), 50% aqueous Me₂NH (3.49 mL), and BuOH (0.5 mL) was heated in a microwave synthesizer at 160° C. for 2 h. The reaction was repeated 1 more time and the reaction mixtures were pooled. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane) to give N²-[(cis-4-benzylaminocyclohexyl)methyl]-N⁴,N⁴,5-trimethylpyridine-2,4-diamine 1-oxide (3.08 g) as a orange oil.

¹H NMR (300 MHz, CDCl₃, δ): 1.51-1.72 (m, 9H), 2.12 (s, 3H), 2.74-2.87 (m, 7H), 3.08-3.16 (m, 2H), 3.77 (s, 2H), 5.95 (s, 1H), 6.51-6.59 (m, 1H), 7.21-7.35 (m, 5H), 7.79 (s, 1H); ESI MS m/z 369 (M⁺+1, 100%).

### Step F: Synthesis of N²-[(cis-4-aminocyclohexyl)methyl]-N⁴N⁴,5-trimethylpyridine-2,4-diamine

A mixture of N²-[(cis-4-benzylaminocyclohexyl)methyl]-N⁴,N⁴,5-trimethylpyridine-2,4-diamine 1-oxide (3.00 g), cyclohexene (16.5 mL), 10% palladium carbon (2.25 g), and 2-propanol (30 mL) was stirred at reflux for 80 h. The mixture was filtered through a pad of celite, concentrated under reduced pressure, and purified by medium-pressure liquid chromatography (NH-silica gel, 50% EtOAc in hexane to 3% MeOH in CHCl₃) to give N²-[(cis-4-aminocyclohexyl)methyl]-N⁴,N⁴,5-trimethylpyridine-2,4-diamine (1.47 g) as a brown oil.

¹H NMR (300 MHz, CDCl₃, δ): 1.44-1.77 (m, 9H), 2.13 (s, 3H), 2.77 (s, 6H), 2.95-3.06 (m, 1H), 3.14-3.20 (m, 2H), 4.24-4.35 (m, 1H), 5.83 (s, 1H), 7.71 (s, 1H); ESI MS m/z 263 (M⁺+1, 100%).

### Step G: Synthesis of 3-chloro-N-[cis-4-({[4-(dimethylamino)-5-methylpyridin-2-yl]amino}methyl)cyclohexyl]-4-fluorobenzamide hydrochloride

To a solution of N²-[(cis-4-aminocyclohexyl)methyl]-N⁴,N⁴,5-trimethylpyridine-2,4-diamine (250 mg) in DMF (3 mL) were added 3-chloro-4-fluorobenzoic acid (200 mg), Et₃N (320 μL), HOBt-H₂O (218 mg), and EDC-HCl (219 mg). The mixture was stirred at ambient temperature for 12 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 14% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-N-[cis-4-({[4-(dimethylamino)-5-methylpyridin-2-yl]amino}methyl)cyclohexyl]-4-fluorobenzamide hydrochloride (140 mg) as a white solid.

¹H NMR (300 MHz, CDCl₃, δ): 1.53-2.10 (m, 9H), 2.19 (s, 3H), 3.06 (s, 6H), 3.16-3.23 (m, 2H), 4.45-4.55 (m, 1H), 5.57 (s, 1H), 7.15-7.28 (m, 2H), 7.36-7.43 (m, 1H), 8.10-8.16 (m, 1H), 8.21 (dd, J=7.1, 2.3 Hz, 1H), 8.34-8.46 (m, 1H), 12.92 (brs, 1H); ESI MS m/z 419 [M (free)⁺+1, 100%].

### Example 32

### 3-Chloro-N-[(cis-4-{[4-(dimethylamino)-5-methylpyridin-2-yl]amino}cyclohexyl)methyl]-4-fluorobenzamide hydrochloride

### Step A: Synthesis of cis-4-[(benzylamino)methyl]cyclohexanamine

A mixture of benzyl ({cis-4-[(tert-butoxycarbonyl)amino]cyclohexyl}methyl)carbamate (200 g), 5% palladium carbon (20.0 g), and MeOH (2 L) was stirred at 50° C. under hydrogen atmosphere for 12 h. The mixture was filtered through a pad of celite and concentrated under reduced pressure to give a pale black solid (140 g). To a solution of the above solid (136 g) in CHCl₃ (1.1 L) were added benzaldehyde (63.1 g) and AcOH (34.0 mL). After the mixture was stirred at ambient temperature for 1 h, NaBH(OAc)₃ (151 g) was added. The mixture was stirred at ambient temperature for 14 h. To the mixture was added 1M aqueous NaOH and the aqueous layer was extracted with CHCl₃ three times. The combined organic layer was dried over MgSO₄, filtered, and concentrated under reduced pressure to give a brown oil (256 g). To the suspension of the above oil (253 g) in EtOAc (759 mL) was added 4 M hydrogen chloride in EtOAc (595 mL) and the mixture was stirred at ambient temperature for 3.25 h and concentrated under reduced pressure. To the residue was added 1M aqueous NaOH and the aqueous layer was extracted with CHCl₃ three times. The combined organic layer was dried over MgSO₄, filtered, and concentrated under reduced pressure. To the residue cooled on an ice-bath was added 3 M aqueous HCl (pH=2). The aqueous layer was washed with CHCl₃ twice. The aqueous layer was alkalized with 2 M aqueous NaOH and extracted with CHCl₃ three times. The combined organic layer was dried over MgSO₄, filtered, concentrated under reduced pressure, and purified by medium-pressure liquid chromatography (NH-silica gel, 33% EtOAc in hexane to 9% MeOH in CHCl₃) to give cis-4-[(benzylamino)methyl]cyclohexanamine (17.0 g) as a orange oil.

¹H NMR (300 MHz, CDCl₃, δ): 1.10-1.69 (m, 11H), 2.55 (d, J=6.9 Hz, 2H), 2.92-3.03 (m, 1H), 3.78 (s, 2H), 7.18-7.38 (m, 5H); ESI MS m/z 219 (M⁺+1, 100%).

### Step B: Synthesis of N-{cis-4-[(benzylamino)methyl]cyclohexyl}-4-chloro-5-methylpyridin-2-amine 1-oxide

A mixture of 2,4-dichloro-5-methylpyridine 1-oxide obtained in step B of example 4 (13.8 g), cis-4-[(benzylamino)methyl]cyclohexanamine (17.0 g), and BuOH (14 mL) was stirred at reflux for 9 h. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 33% EtOAc in hexane to 5% MeOH in CHCl₃) to give N-{cis-4-[(benzylamino)methyl]cyclohexyl}-4-chloro-5-methylpyridin-2-amine 1-oxide (7.13 g) as a brown oil.

¹H NMR (200 MHz, CDCl₃, δ): 1.15-1.93 (m, 9H), 2.18 (s, 3H), 2.47-2.61 (m, 2H), 3.50-3.69 (m, 1H), 3.78 (s, 2H), 6.55 (s, 1H), 6.76-6.88 (m, 1H), 7.18-7.40 (m, 5H), 7.98 (s, 1H); ESI MS m/z 360 (M⁺+1, 100%).

### Step C: Synthesis of N²-{cis-4-[(benzylamino)methyl]cyclohexyl}-N⁴,N⁴,5-trimethylpyridine-2,4-diamine 1-oxide

A mixture of N-{cis-4-[(benzylamino)methyl]cyclohexyl}-4-chloro-5-methylpyridin-2-amine 1-oxide (1.77 g), 50% aqueous Me₂NH (3.10 mL), and BuOH (9.45 mL) was heated in a microwave synthesizer at 160° C. for 2 h. The reaction was repeated 3 more times and the reaction mixtures were pooled. The mixture was diluted with CHCl₃ and poured into aqueous saturated NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% to 100% EtOAc in hexane) to give N²-{cis-4-[(benzylamino)methyl]cyclohexyl}-N⁴,N⁴,5-trimethylpyridine-2,4-diamine 1-oxide (4.54 g) as a orange oil.

¹H NMR (300 MHz, CDCl₃, δ): 1.32-1.92 (m, 9H), 2.12 (s, 3H), 2.54 (d, J=6.5 Hz, 2H), 2.76 (s, 6H), 3.56-3.70 (m, 1H), 3.78 (s, 2H), 5.95 (s, 1H), 6.71-6.77 (m, 1H), 7.21-7.34 (m, 5H), 7.80 (s, 1H); ESI MS m/z 369 (M⁺+1, 90%), 737 (M⁺+369, 100%).

### Step D: Synthesis of N²-[cis-4-(aminomethyl)cyclohexyl]-N⁴,N⁴,5-trimethylpyridine-2,4-diamine

A mixture of N²-{cis-4-[(benzylamino)methyl]cyclohexyl}-N⁴,N⁴,5-trimethylpyridine-2,4-diamine 1-oxide (4.50 g), cyclohexene (24.7 mL), 10% palladium carbon (3.37 g), and 2-propanol (45.0 mL) was stirred at reflux for 80 h. The mixture was filtered through a pad of celite, concentrated under reduced pressure, and purified by medium-pressure liquid chromatography (NH-silica gel, 50% EtOAc in hexane to 3% MeOH in CHCl₃) to give N²-[cis-4-(aminomethyl)cyclohexyl]-N⁴,N⁴,5-trimethylpyridine-2,4-diamine (1.04 g) as a brown oil.

¹H NMR (300 MHz, CDCl₃, δ): 1.18-1.91 (m, 9H), 2.13 (s, 3H), 2.59 (d, J=6.2 Hz, 2H), 2.77 (s, 6H), 3.77-3.88 (m, 1H) 4.38-4.49 (m, 1H) 5.84 (s, 1H) 7.71 (s, 1H); ESI MS m/z 263 (M⁺+1, 100%).

### Step E: Synthesis of 3-chloro-N-[(cis-4-{[4-(dimethylamino)-5-methylpyridin-2-yl]amino}cyclohexyl)methyl]-4-fluorobenzamide hydrochloride

To a solution of N²-[cis-4-(aminomethyl)cyclohexyl]-N⁴,N⁴,5-trimethylpyridine-2,4-diamine (250 mg) in DMF (3 mL) were added 3-chloro-4-fluorobenzoic acid (200 mg), Et₃N (320 μL), HOBt-H₂O (218 mg), and EDC-HCl (219 mg). The mixture was stirred at ambient temperature for 12 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 14% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-N-[(cis-4-{[4-(dimethylamino)-5-methylpyridin-2-yl]amino}cyclohexyl)methyl]-4-fluorobenzamide hydrochloride (274 mg) as a white solid.

¹H NMR (300 MHz, CDCl₃, δ): 1.52-1.98 (m, 9H), 2.19 (s, 3H), 3.04 (s, 6H), 3.43-3.50 (m, 2H), 3.70-3.79 (m, 1H), 5.65 (s, 1H), 7.12-7.24 (m, 3H), 7.97-8.05 (m, 1H), 8.11 (dd, J=7.0, 2.2 Hz, 1H), 8.39 (d, J=8.4 Hz, 1H), 13.15 (brs, 1H); ESI MS m/z 419 [M (free)⁺+1, 100%].

### Example 33

### 3-Chloro-4-fluoro-N-{cis-4-[(4-methyl-6,7-dihydro-5H-cyclopenta[b]pyridin-2-yl)amino]cyclohexyl}benzamide hydrochloride

A mixture of cyclopentanone (55.0 g), ethyl acetoacetate (85.1 g), and AcONH₄ (50.4 g) was stirred at reflux for 9.5 h and stirred at ambient temperature for 8 h. The precipitate was collected by filtration, washed with water and hexane, and dried at 80° C. to give a white solid (17.7 g). A mixture of the above solid (4.00 g) in POCl₃ (3.00 mL) and N,N-dimethylaniline (3.73 g) was stirred at reflux for 2 h. After cooling, the mixture was poured into EtOAc and cold water and the organic layer was separated. The aqueous layer was extracted with EtOAc twice. The combined organic layer was dried over MgSO₄, filtered, and concentrated under reduced pressure to give 2-chloro-4-methyl-6,7-dihydro-5H-cyclopenta[b]pyridine including N,N-dimethylaniline (1.95 g) as a brown oil. A mixture of the above oil (650 mg), N-(cis-4-aminocyclohexyl)-3-chloro-4-fluorobenzamide obtained in step A of example 1 (1.26 g), and BuOH (1 mL) was heated in a microwave synthesizer at 230° C. for 30 min. The mixture was poured into saturated aqueous NaHCO₃ and the aqueous layer was extracted with CHCl₃ three times. The combined organic layer was dried over MgSO₄, filtrated, concentrated under reduced pressure, and purified by medium-pressure liquid chromatography (NH-silica gel, 40% to 50% EtOAc in hexane and silica gel, 3% to 10% MeOH in CHCl₃). To a solution of the above purified material in EtOAc (5 mL) was added 4 M hydrogen chloride in EtOAc (10 mL). The mixture was stirred at ambient temperature for 4 h. The precipitate was collected by filtration, washed with Et₂O, and dried at 80° C. under reduced pressure to give 3-chloro-4-fluoro-N-{cis-4-[(4-methyl-6,7-dihydro-5H-cyclopenta[b]pyridin-2-yl)amino]cyclohexyl}benzamide hydrochloride (30.0 mg) as a white solid.

¹H NMR (300 MHz, CDCl₃, δ): 1.66-2.06 (m, 8H), 2.14-2.28 (m, 2H), 2.32 (s, 3H), 2.79 (t, J=7.4 Hz, 2H), 3.09 (t, J=7.6 Hz, 2H), 3.72-3.83 (m, 1H), 4.05-4.20 (m, 1H), 6.34 (s, 1H), 6.66-6.79 (m, 1H), 7.18 (t, J=8.6 Hz, 1H), 7.67-7.77 (m, 1H), 7.95 (dd, J=7.1, 1.9 Hz, 1H), 8.50-8.61 (m, 1H); ESI MS m/z 402 [M (free)⁺+1, 100%].

### Example 34

### 3-Chloro-4-fluoro-N-{cis-4-[(4-methyl-5,6,7,8-tetrahydroquinolin-2-yl)amino]cyclohexyl}benzamide hydrochloride

### Step A: Synthesis of 4-methyl-5,6,7,8-tetrahydroquinolin-2-ol

A mixture of cyclohexanone (60.3 g), ethyl acetoacetate (79.9 g), and AcONH₄ (47.4 g) was stirred at reflux for 12 h and at ambient temperature for 12 h. To the mixture was added water (300 mL) and the mixture was stirred at ambient temperature for 4 h. The precipitate was collected by filtration, washed with water and hexane, and dried at 80° C. To the material was added a mixture of hexane (150 mL) and EtOAc (150 mL) and the suspension was stirred at ambient temperature for 30 min. The precipitate was collected by filtration and dried at 80° C. to give 4-methyl-5,6,7,8-tetrahydroquinolin-2-ol (3.92 g) as a pale brown solid.

¹H NMR (200 MHz, CDCl₃, δ): 1.67-1.87 (m, 4H), 2.11 (s, 3H), 2.33-2.47 (m, 2H), 2.60-2.75 (m, 2H), 6.25 (s, 1H), 12.63 (brs, 1H); ESI MS m/z 164 (M⁺+1, 100%).

### Step B: Synthesis of 2-chloro-4-methyl-5,6,7,8-tetrahydroquinoline

A mixture of 4-methyl-5,6,7,8-tetrahydroquinolin-2-ol (1.96 g), POCl₃ (2.35 mL), and N,N-dimethylaniline (1.66 g) was stirred at reflux for 1 h. After cooling, the mixture was poured into a mixture of EtOAc (100 mL) and cold water (100 mL) and the organic layer was separated. The aqueous layer was extracted with EtOAc twice. The combined organic layer was washed with aqueous saturated NaCl, dried over MgSO₄, filtered, concentrated under reduced pressure, and purified by medium-pressure liquid chromatography (silica gel, 5% to 20% EtOAc in hexane) to give 2-chloro-4-methyl-5,6,7,8-tetrahydroquinoline (1.44 g) as a pale yellow oil.

¹H NMR (300 MHz, CDCl₃, δ): 1.74-1.90 (m, 4H), 2.19 (s, 3H), 2.50-2.65 (m, 2H), 2.78-2.93 (m, 2H), 6.94 (s, 1H); ESI MS m/z 182 (M⁺+1, 100%).

### Step C: Synthesis of 3-chloro-4-fluoro-N-{cis-4-[(4-methyl-5,6,7,8-tetrahydroquinolin-2-yl)amino]cyclohexyl}benzamide hydrochloride

A mixture of 2-chloro-4-methyl-5,6,7,8-tetrahydroquinoline (500 mg), N-(cis-4-aminocyclohexyl)-3-chloro-4-fluorobenzamide obtained in step A of example 1 (819 mg), and BuOH (1 mL) was heated in a microwave synthesizer at 240° C. for 1 h. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 6% to 25% EtOAc in hexane and silica gel, 3% to 9% MeOH in CHCl₃). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-{cis-4-[(4-methyl-5,6,7,8-tetrahydroquinolin-2-yl)amino]-cyclohexyl}benzamide hydrochloride (332 mg) as a white solid.

¹H NMR (300 MHz, CDCl₃, δ): 1.69-2.04 (m, 12H), 2.29 (s, 3H), 2.44-2.55 (m, 2H), 2.83-2.94 (m, 2H), 3.74-3.83 (m, 1H), 4.05-4.19 (m, 1H), 6.41 (s, 1H), 6.79-6.89 (m, 1H), 7.13-7.23 (m, 1H), 7.69-7.78 (m, 1H), 7.93-8.00 (m, 1H), 8.46-8.56 (m, 1H), 14.15 (brs, 1H); ESI MS m/z 236 [M (free)⁺−179, 100%], 416 [M (free)⁺+1, 95%].

### Example 35

### 3-Chloro-4-fluoro-N-{cis-4-[(4-methylpyridin-2-yl)amino]cyclohexyl}benzamide hydrochloride

Using the procedure for the step B of example 1, the title compound was obtained.

¹H NMR (300 MHz, CDCl₃, δ): 1.74-2.01 (m, 8H), 2.43 (s, 3H), 3.77-3.87 (m, 1H), 4.05-4.21 (m, 1H), 6.54-6.61 (m, 2H), 6.68 (d, J=8.7 Hz, 1H), 7.18 (t, J=8.6 Hz, 1H), 7.60 (d, J=6.4 Hz, 1H), 7.68-7.75 (m, 1H), 7.95 (dd, J=7.0, 2.2 Hz, 1H), 9.05 (d, J=7.9 Hz, 1H); ESI MS m/z 362 [M (free)⁺+1, 100%].

### Example 36

### 3-Chloro-N-(cis-4-{[4-(dimethylamino)pyridin-2-yl]amino}cyclohexyl)-4-fluorobenzamide hydrochloride

### Step A: Synthesis of 2,4-dichloropyridine

A suspension of 2,4-dihydroxypyridine (31.3 g) and N,N-dimethylaniline (38.0 mL) in POCl₃ (53.0 mL) was stirred at reflux for 40 min and cooled to ambient temperature. The reaction mixture was poured into ice water (1 L) below 10° C. The aqueous layer was extracted with CHCl₃ three times. The combined organic layer was dried over Na₂SO₄, filtrated, and concentrated under reduced pressure to give 2,4-dichloropyridine (45.9 g) as a brown solid.

¹H NMR (300 MHz, CDCl₃, δ): 7.24-7.27 (m, 1H), 7.38 (dd, J=1.9, 0.5 Hz, 1H), 8.32 (d, J=5.4 Hz, 1H); CI MS m/z 148 (M⁺+1, 100%).

### Step B: Synthesis of 2,4-dichloropyridine 1-oxide

To a solution of 2,4-dichloropyridine (7.00 g) in CHCl₃ (50 mL) cooled on an ice-bath was added a solution of mCPBA (12.6 g) in CHCl₃ (100 mL) dropwise. The mixture was stirred at 0° C. for 30 min, at ambient temperature for 20 h, and at 50° C. for 4 h. After cooling on an ice-bath, aqueous saturated Na₂S₂O₃ was added. The aqueous layer was extracted with CHCl₃ three times. The combined organic layer was washed with aqueous 1 M NaOH and brine. The resulting organic layer was dried over MgSO₄, filtrated, concentrated under reduced pressure, and purified by medium-pressure liquid chromatography (silica gel, 50% to 99% EtOAc in hexane) to give 2,4-dichloropyridine 1-oxide (4.71 g) as a yellow oil.

¹H NMR (300 MHz, CDCl₃, δ): 7.21 (dd, J=7.1, 2.9 Hz, 1H), 7.52 (d, J=2.8 Hz, 1H), 8.26 (d, J=7.0 Hz, 1H); ESI MS m/z 164 (M⁺+1, 30%), 186 (M⁺+23, 100%).

### Step C: Synthesis of cis-N-benzyl-N′-(4-chloro-1-oxidopyridin-2-yl)cyclohexane-1,4-diamine

A mixture of 2,4-dichloropyridine 1-oxide (3.00 g) and cis-N-benzyl-cyclohexane-1,4-diamine obtained in step C of example 4 (5.60 g) in BuOH (6 mL) was stirred at reflux for 4 h. The reaction mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 10% to 99% EtOAc in hexane) to give cis-N-benzyl-N′-(4-chloro-1-oxidopyridin-2-yl)cyclohexane-1,4-diamine (4.39 g) as a yellow oil.

¹H NMR (300 MHz, CDCl₃, δ): 1.53-1.80 (m, 6H), 1.81-1.97 (m, 2H), 2.73-2.80 (m, 1H), 3.42-3.55 (m, 1H), 3.80 (s, 2H), 6.50 (dd, J=6.9, 2.7 Hz, 1H), 6.55 (d, J=2.6 Hz, 1H), 7.04 (d, J=8.6 Hz, 1H), 7.21-7.35 (m, 5H), 8.02 (d, J=7.0 Hz, 1H); ESI MS m/z 332 (M⁺+1, 100%).

### Step D: Synthesis of N²-[cis-4-(benzylamino)cyclohexyl]-N⁴,N⁴-dimethylpyridine-2,4-diamine 1-oxide

A mixture of cis-N-benzyl-N′-(4-chloro-1-oxidopyridin-2-yl)cyclohexane-1,4-diamine (2.02 g), aqueous 50% Me₂NH (3.29 mL), and BuOH (0.5 mL) was heated in a microwave synthesizer at 160° C. for 1.5 h. The reaction mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 50% EtOAc in hexane to 10% MeOH in CHCl₃) to give N²-[cis-4-(benzylamino)cyclohexyl]-N⁴,N⁴-dimethylpyridine-2,4-diamine 1-oxide (4.37 g) as a brown oil.

¹H NMR (300 MHz, CDCl₃, δ): 1.50-1.99 (m, 8H), 2.68-2.78 (m, 1H), 2.99 (s, 6H), 3.42-3.54 (m, 1H), 3.81 (s, 2H), 5.59 (d, J=3.1 Hz, 1H), 5.88 (dd, J=7.5, 3.1 Hz, 1H), 6.87 (d, J=8.1 Hz, 1H), 7.21-7.37 (m, 5H), 7.83 (d, J=7.5 Hz, 1H); ESI MS m/z 341 (M⁺+1, 100%).

### Step E: Synthesis of 3-chloro-N-(cis-4-{[4-(dimethylamino)pyridin-2-yl]amino}cyclohexyl)-4-fluorobenzamide hydrochloride

To a solution of N²-[cis-4-(benzylamino)cyclohexyl]-N⁴,N⁴-dimethylpyridine-2,4-diamine 1-oxide (2.01 g) in MeOH (20 mL) was added 10% Pd/C (200 mg). The mixture was stirred at 50° C. under hydrogen atmosphere for 92.5 h and filtrated through a pad of celite with MeOH. The filtrate was concentrated under reduced pressure to give crude N²-(cis-4-aminocyclohexyl)-N⁴,N⁴-dimethylpyridine-2,4-diamine (1.49 g). To a solution of the above material (450 mg) and 3-chloro-4-fluorobenzoic acid (402 mg) in DMF (4.5 mL) were added EDC-HCl (442 mg), HOBt-H₂O (389 mg), and Et₃N (0.64 mL). The mixture was stirred at ambient temperature for 2 days, diluted with CHCl₃, and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 33% to 99% EtOAc in hexane and silica gel, 1% to 30% MeOH in CHCl₃) to give 3-chloro-N-(cis-4-{[4-(dimethylamino)pyridin-2-yl]amino}cyclohexyl)-4-fluorobenzamide. To a solution of the above material in EtOAc (5 mL) was added 4 M hydrogen chloride in EtOAc (0.09 mL). The mixture was stirred at ambient temperature for 4 h and concentrated under reduced pressure. The residue was suspended in Et₂O and the suspension was stirred at ambient temperature for 4 h. The precipitate was collected by filtration, washed with Et₂O, and dried at 80° C. under reduced pressure to give 3-chloro-N-(cis-4-{[4-(dimethylamino)pyridin-2-yl]amino}cyclohexyl)-4-fluorobenzamide hydrochloride (42 mg) as a pale yellow powder.

¹H NMR (300 MHz, CDCl₃, δ): 1.66-2.03 (m, 8H), 3.14 (s, 6H), 3.69-3.77 (m, 1H), 4.04-4.19 (m, 1H), 5.44 (brs, 1H), 6.09 (dd, J=7.2, 1.9 Hz, 1H), 6.80 (d, J=8.6 Hz, 1H), 7.18 (t, J=8.6 Hz, 1H), 7.39 (dd, J=7.2, 6.0 Hz, 1H), 7.69-7.77 (m, 1H), 7.96 (dd, J=7.0, 2.2 Hz, 1H), 8.28 (d, J=7.9 Hz, 1H), 13.15 (brs, 1H); ESI MS m/z 87 [M (free)⁺−303, 100%], 391 [M (free)⁺+1, 55%].

### Example 37

### N-(cis-4-{[4-(Dimethylamino)pyridin-2-yl]amino}cyclohexyl)-3,4,5-trifluorobenzamide hydrochloride

Using the procedure for the step E of example 36, the title compound was obtained.

¹H NMR (300 MHz, CDCl₃, δ): 1.63-2.09 (m, 8H), 3.14 (s, 6H), 3.69-3.79 (m, 1H), 4.02-4.18 (m, 1H), 5.45 (d, J=2.5 Hz, 1H), 6.09 (dd, J=7.5, 2.5 Hz, 1H), 7.11 (d, J=8.2 Hz, 1H), 7.40 (d, J=7.5 Hz, 1H), 7.51-7.63 (m, 2H), 8.24 (d, J=8.6 Hz, 1H); ESI MS m/z 393 [M (free)⁺+1, 100%].

### Example 38

### 3-Chloro-N-(cis-4-{[6-(dimethylamino)pyridin-2-yl]amino}cyclohexyl)-4-fluorobenzamide hydrochloride

### Step A: Synthesis of 2,6-dibromopyridine 1-oxide

To a suspension of 2,6-dibromopyridine (10.0 g) in Ac₂O (27 mL) cooled on an ice-bath was added aqueous 30% H₂O₂ (5.7 g) dropwise. The mixture was stirred at 0° C. for 15 min and at ambient temperature for 1.5 h and cooled on an ice-bath. To the mixture was added TFA (27 mL) dropwise over 30 min. The mixture was stirred at ambient temperature for 2.5 h and at 50° C. for 2.5 h. To the mixture was added aqueous 30% H₂O₂ (53.5 g) in small portions over time and the mixture was stirred at ambient temperature for 90 h and at 60° C. for 18 h and poured into cooled aqueous 2.5 M NaOH (500 mL). The aqueous layer was extracted with CHCl₃ three times. The combined organic layer was dried over MgSO₄, filtrated, concentrated under reduced pressure, and purified by medium-pressure liquid chromatography (silica gel, 50% to 99% EtOAc in hexane) to give 2,6-dibromopyridine 1-oxide (4.79 g) as a pale yellow solid.

¹H NMR (300 MHz, CDCl₃, δ): 6.90-6.97 (m, 1H), 7.65 (d, J=8.2 Hz, 2H); ESI MS m/z 252 (M⁺+1, 20%), 276 (M⁺+25, 100%).

### Step B: Synthesis of cis-N-benzyl-N′-(6-bromo-1-oxidopyridin-2-yl)cyclohexane-1,4-diamine

A suspension of 2,6-dibromopyridine 1-oxide (2.00 g) and cis-N-benzyl-cyclohexane-1,4-diamine obtained in step C of example 4 (1.62 g) in BuOH (5 mL) was stirred at reflux for 5 h and at ambient temperature for 19 h. The mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 15% to 99% EtOAc in hexane) to give cis-N-benzyl-N′-(6-bromo-1-oxidopyridin-2-yl)cyclohexane-1,4-diamine (1.89 g) as a yellow oil.

¹H NMR (300 MHz, CDCl₃, δ): 1.57-1.97 (m, 8H), 2.69-2.79 (m, 1H), 3.46-3.59 (m, 1H), 3.80 (s, 2H), 6.51 (dd, J=8.2, 1.9 Hz, 1H), 6.87-6.91 (m, 1H), 6.98 (t, J=8.2 Hz, 1H), 7.16-7.37 (m, 5H); ESI MS m/z 376 (M⁺+1, 99%), 378 (M⁺+3, 100%).

### Step C: Synthesis of N′-[cis-4-(benzylamino)cyclohexyl]-N,N-dimethylpyridine-2,6-diamine 1-oxide

A mixture of cis-N-benzyl-N′-(6-bromo-1-oxidopyridin-2-yl)cyclohexane-1,4-diamine (1.38 g), aqueous 50% Me₂NH (1.98 mL), and BuOH (0.5 mL) was heated in a microwave synthesizer at 170° C. for 1.5 h. The mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 30% EtOAc in hexane to 10% MeOH in CHCl₃) to give N′-[cis-4-(benzylamino)cyclohexyl]-N,N-dimethylpyridine-2,6-diamine 1-oxide (1.02 g) as a brown oil.

¹H NMR (300 MHz, CDCl₃, δ): 1.55-1.97 (m, 8H), 2.68-2.77 (m, 1H), 3.03 (s, 6H), 3.44-3.58 (m, 1H), 3.80 (s, 2H), 6.08 (dd, J=8.2, 1.7 Hz, 1H), 6.16 (dd, J=8.4, 1.6 Hz, 1H), 7.05 (t, J=8.2 Hz, 1H), 7.20-7.37 (m, 5H); ESI MS m/z 341 (M⁺+1, 100%).

### Step D: Synthesis of N′-(cis-4-aminocyclohexyl)-N,N-dimethylpyridine-2,6-diamine

To a solution of N′-[cis-4-(benzylamino)cyclohexyl]-N,N-dimethylpyridine-2,6-diamine 1-oxide (960 mg) in MeOH (10 mL) was added 10% Pd/C (96.0 mg). The mixture was stirred at 50° C. under hydrogen atmosphere for 118 h and filtered through a pad of celite. The filtrate was concentrated under reduced pressure to give N′-(cis-4-aminocyclohexyl)-N,N-dimethylpyridine-2,6-diamine (620 mg) as a yellow oil.

¹H NMR (300 MHz, CDCl₃, δ): 1.36-1.88 (m, 8H), 2.81-2.92 (m, 1H), 3.00 (s, 6H), 3.71-3.81 (m, 1H), 4.30-4.44 (m, 1H), 5.67 (d, J=7.8 Hz, 1H), 5.81 (dd, J=8.1, 0.5 Hz, 1H), 7.23 (t, J=7.9 Hz, 1H); ESI MS m/z 235 (M⁺+1, 100%).

### Step E: Synthesis of 3-chloro-N-(cis-4-{[6-(dimethylamino)pyridin-2-yl]amino}cyclohexyl)-4-fluorobenzamide hydrochloride

To a solution of N′-(cis-4-aminocyclohexyl)-N,N-dimethylpyridine-2,6-diamine (300 mg) and 3-chloro-4-fluorobenzoic acid (268 mg) in DMF (3.0 mL) were added EDC-HCl (294 mg), HOBt-H₂O (259 mg), and Et₃N (0.43 mL). The mixture was stirred at ambient temperature for 1 day, diluted with CHCl₃, and poured into aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 20% EtOAc in hexane and silica gel, CHCl₃ to 10% MeOH in CHCl₃) to give 3-chloro-N-(cis-4-{[6-(dimethylamino)pyridin-2-yl]amino}cyclohexyl)-4-fluorobenzamide. To a solution of the above material in EtOAc (5 mL) was added 4 M hydrogen chloride in EtOAc (0.38 mL). The mixture was stirred at ambient temperature for 4 h and concentrated under reduced pressure. The residue was suspended in Et₂O and the suspension was stirred at ambient temperature for 4 h. The precipitate was collected by filtration, washed with Et₂O, and dried at 80° C. under reduced pressure to give 3-chloro-N-(cis-4-{[6-(dimethylamino)pyridin-2-yl]amino}cyclohexyl)-4-fluorobenzamide hydrochloride (260 mg) as a powder.

¹H NMR (300 MHz, CDCl₃, δ): 1.67-1.85 (m, 2H), 1.86-2.12 (m, 6H), 3.29 (s, 6H), 3.67-3.76 (m, 1H), 4.05-4.20 (m, 1H), 5.74-5.82 (m, 2H), 6.68 (d, J=8.4 Hz, 1H), 7.18 (t, J=8.6 Hz, 1H), 7.48 (t, J=8.5 Hz, 1H), 7.68-7.76 (m, 1H), 7.95 (dd, J=7.1, 2.3 Hz, 1H), 9.08 (d, J=8.2 Hz, 1H), 12.75 (brs, 1H); ESI MS m/z 391 [M (free)⁺+1, 100%].

### Example 39

### 3-Chloro-4-fluoro-N-[cis-4-(5,6,7,8-tetrahydroquinolin-2-ylamino)cyclohexyl]benzamide hydrochloride

### Step A: Synthesis of 5,6,7,8-tetrahydroquinolin-2(1H)-one

To conc. H₂SO₄ (200 mL) cooled on an ice-bath was added 2-oxo-1-cyclohexane propionitrile (49.8 g) dropwise and the mixture was stirred at 0° C. for 10 min and at ambient temperature for 17 h and poured into ice water (1 L). The aqueous layer was washed with CHCl₃ and the resulting aqueous layer was neutralized with aqueous 25-50% NaOH on an ice-bath and extracted with CHCl₃ three times. The combined organic layer was washed with brine, dried over MgSO₄, filtrated, and concentrated under reduced pressure to give 5,6,7,8-tetrahydroquinolin-2(1H)-one (42.3 g) as a pale yellow solid.

¹H NMR (300 MHz, CDCl₃, δ): 1.63-1.85 (m, 4H), 2.48 (t, J=5.4 Hz, 2H), 2.67 (t, J=5.7 Hz, 2H), 6.37 (d, J=9.2 Hz, 1H), 7.19 (d, J=9.2 Hz, 1H), 12.65 (brs, 1H); ESI MS m/z 150 (M⁺+1, 100%).

### Step B: Synthesis of 2-chloro-5,6,7,8-tetrahydroquinoline

A suspension of 5,6,7,8-tetrahydroquinolin-2(1H)-one (19.6 g) and N,N-dimethylaniline (18.4 mL) in POCl₃ (13.5 mL) was stirred at reflux for 1 h. After cooling, the mixture was poured into ice water (250 mL) below 10° C. The aqueous layer was extracted with CHCl₃ three times. The combined organic layer was dried over MgSO₄, filtrated, concentrated under reduced pressure, and purified by medium-pressure liquid chromatography (flash silica gel, 6% to 9% EtOAc in hexane) to give 2-chloro-5,6,7,8-tetrahydroquinoline (2.31 g) as a colorless oil.

¹H NMR (300 MHz, CDCl₃, δ): 1.75-1.92 (m, 4H), 2.73 (t, J=6.2 Hz, 2H), 2.89 (t, J=6.3 Hz, 2H), 7.05 (d, J=8.1 Hz, 1H), 7.31 (d, J=7.9 Hz, 1H); ESI MS m/z 168 (M⁺+1, 100%).

### Step C: Synthesis of 3-chloro-4-fluoro-N-[cis-4-(5,6,7,8-tetrahydroquinolin-2-ylamino) cyclohexyl]benzamide hydrochloride

A mixture of 2-chloro-5,6,7,8-tetrahydroquinoline (400 mg), N-(cis-4-aminocyclohexyl)-3-chloro-4-fluorobenzamide obtained in step A of example 1 (711 mg), and BuOH (3.0 mL) was heated in a microwave synthesizer at 230° C. for 2.5 h. The mixture was diluted with CHCl₃ and added aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 10% to 99% EtOAc in hexane and flash silica gel, 1% to 3% MeOH in CHCl₃) to give 3-chloro-4-fluoro-N-[cis-4-(5,6,7,8-tetrahydroquinolin-2-ylamino)cyclohexyl]benzamide. To a solution of the above material in EtOAc (5 mL) was added 4 M hydrogen chloride in EtOAc (0.21 mL). The mixture was stirred at ambient temperature for 4 h and concentrated under reduced pressure. The residue was suspended in Et₂O and the suspension was stirred at ambient temperature for 4 h. The precipitate was collected by filtration, washed with Et₂O, and dried at 80° C. under reduced pressure to give 3-chloro-4-fluoro-N-[cis-4-(5,6,7,8-tetrahydroquinolin-2-ylamino)cyclohexyl] benzamide hydrochloride (132 mg) as a colorless powder.

¹H NMR (300 MHz, CDCl₃, δ): 1.71-2.03 (m, 12H), 2.61 (t, J=5.3 Hz, 2H), 2.84-2.95 (m, 2H), 3.73-3.85 (m, 1H), 4.01-4.23 (m, 1H), 6.56 (d, J=8.8 Hz, 1H), 6.70 (d, J=8.4 Hz, 1H), 7.18 (t, J=8.8 Hz, 1H), 7.52 (d, J=9.2 Hz, 1H), 7.67-7.76 (m, 1H), 7.94 (dd, J=7.0, 2.2 Hz, 1H), 8.73 (d, J=7.9 Hz, 1H); ESI MS m/z 402 [M (free)⁺+1, 100%].

### Example 40

### 3,4,5-Trifluoro-N-[cis-4-(5,6,7,8-tetrahydroquinolin-2-ylamino)cyclohexyl]benzamide hydrochloride

Using the procedure for the step C of example 39, the title compound was obtained.

¹H NMR (300 MHz, CDCl₃, δ): 1.70-2.01 (m, 12H), 2.62 (t, J=5.8 Hz, 2H), 2.90 (t, J=6.1 Hz, 2H), 3.77-3.85 (m, 1H), 4.03-4.18 (m, 1H), 6.56 (d, J=9.0 Hz, 1H), 6.87 (d, J=8.4 Hz, 1H), 7.48-7.59 (m, 3H), 8.72 (d, J=8.7 Hz, 1H); ESI MS m/z 404 [M (free)⁺+1, 100%].

### Example 41

### 3-Chloro-4-fluoro-N-{cis-4-[(4-nitropyridin-2-yl)amino]cyclohexyl}benzamide hydrochloride

A mixture of 2-chloro-4-nitropyridine (2.54 g) and N-(cis-4-aminocyclohexyl)-3-chloro-4-fluorobenzamide obtained in step A of example 1 (4.77 g) in BuOH (6.0 mL) was stirred at reflux for 6.5 h and at ambient temperature for 22 h. The mixture was diluted with CHCl₃ and added aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 9% to 99% EtOAc in hexane and silica gel, 1% MeOH in CHCl₃) to give 3-chloro-4-fluoro-N-{cis-4-[(4-nitropyridin-2-yl)amino]cyclohexyl}-benzamide (549 mg). To a solution of the above material (50 mg) in EtOAc (4.0 mL) was added 4 M hydrogen chloride in EtOAc (0.06 mL). The mixture was stirred at ambient temperature for 3 h and concentrated under reduced pressure. The residue was suspended in Et₂O and the suspension was stirred at ambient temperature for 12 h. The precipitate was collected by filtration, washed with Et₂O, and dried at 80° C. under reduced pressure to give 3-chloro-4-fluoro-N-{cis-4-[(4-nitropyridin-2-yl)amino]cyclohexyl}benzamide hydrochloride (35 mg) as a yellow powder.

¹H NMR (300 MHz, CDCl₃, δ): 1.81-2.10 (m, 8H), 3.92-4.02 (m, 1H), 4.08-4.23 (m, 1H), 6.49 (d, J=8.4 Hz, 1H), 7.20 (t, J=8.6 Hz, 1H), 7.40 (dd, J=6.7, 2.0 Hz, 1H), 7.57 (d, J=2.0 Hz, 1H), 7.66-7.73 (m, 1H), 7.92 (dd, J=7.0, 2.2 Hz, 1H), 8.07 (d, J=6.7 Hz, 1H), 9.62 (brs, 1H); ESI MS m/z 393 [M (free)⁺+1, 100%].

### Example 42

### 3,4,5-Trifluoro-N-{cis-4-[(4-nitropyridin-2-yl)amino]cyclohexyl}benzamide hydrochloride

Using the procedure for example 41, the title compound was obtained.

¹H NMR (300 MHz, CDCl₃, δ): 1.81-2.09 (m, 8H), 3.94-4.03 (m, 1H), 4.09-4.22 (m, 1H), 6.62 (d, J=7.8 Hz, 1H), 7.40 (dd, J=6.8, 1.9 Hz, 1H), 7.45-7.59 (m, 3H), 8.08 (d, J=6.7 Hz, 1H), 9.49 (brs, 1H); ESI MS m/z 395 [M (free)⁺+1, 100%].

### Example 43

### 3-Chloro-N-{cis-4-[(5,6-dimethylpyridin-2-yl)amino]cyclohexyl}-4-fluorobenzamide hydrochloride

### Step A: Synthesis of sodium (1E)-2-methyl-3-oxobut-1-en-1-olate

To a solution of 25% sodium methoxide in MeOH (60.5 mL) in ether (360 mL) cooled on an ice-bath were added a mixture of 2-butanone (20.0 g) and ethyl formate (22.4 g) under nitrogen atmosphere below 2-3° C. over 15 min. The mixture was stirred at 0° C. for 30 min and at ambient temperature for 21 h. The precipitate was collected by filtration, washed with Et₂O, and dried at 80° C. under reduced pressure to give sodium (1E)-2-methyl-3-oxobut-1-en-1-olate (20.7 g) as a colorless powder.

¹H NMR (300 MHz, DMSO-d₆, δ): 1.39 (s, 3H), 1.88 (s, 3H), 9.07 (s, 1H); ESI MS m/z 99 (M⁺−1, 100%), 101 (M⁺+1, 5%).

### Step B: Synthesis of 5,6-dimethyl-2-oxo-1,2-dihydropyridine-3-carbonitrile

To a solution of sodium (1E)-2-methyl-3-oxobut-1-en-1-olate (20.0 g) in H₂O (310 mL) was added 2-cyanoacetamide (14.5 g). This solution (15.5 mL) was distributed to two flasks and to the one flask was added piperidinium acetate (7.35 g) and to another flask were added piperidine (5.0 mL) and acetic acid (2.9 mL). Both solutions were stirred at reflux for 30 min. To the remaining solution prepared first were added piperidine (90.5 mL). After cooling, to the mixture was added acetic acid (52.3 mL). All reaction mixtures were stirred at reflux for 14 h and cooled down. At 60° C., to each solution was added acetic acid (1.25 mL, 1.25 mL, 22.5 mL) the mixture was stirred to ambient temperature. The precipitate was collected by filtration, washed with H₂O, dried under reduced pressure, and suspended in 50% MeOH in CHCl₃. The mixture was heated by a dryer and filtered. The insoluble material was suspended in 50% MeOH in CHCl₃ and the mixture was stirred at reflux for 1 h and filtered. The two filtrates were concentrated under reduced pressure to give 5,6-dimethyl-2-oxo-1,2-dihydropyridine-3-carbonitrile (5.95 g) as a yellow solid.

¹H NMR (300 MHz, DMSO-d₆, δ): 1.98 (s, 3H), 2.23 (s, 3H), 7.94 (s, 1H), 12.43 (brs, 1H); ESI MS m/z 149 (M⁺+1, 55%), 171 (M⁺+23, 100%).

### Step C: Synthesis of 5,6-dimethylpyridin-2(1H)-one

To a suspension of 5,6-dimethyl-2-oxo-1,2-dihydropyridine-3-carbonitrile (5.90 g) in H₂O (145 mL) was added conc. HCl (145 mL) dropwise and the mixture was stirred at ambient temperature for 15 min and at reflux for 60.5 h and concentrated under reduced pressure. The residue was suspended with CHCl₃ (150 mL) and MeOH (7.5 mL) and the mixture was heated at 65° C. on a water bath and filtered. The insoluble material was suspended in CHCl₃ (100 mL) and MeOH (5 mL) and the mixture was heated at 65° C. on a water bath and filtered. The combined filtrate was concentrated under reduced pressure. To the residue were added MeOH (75 mL) and K₂CO₃ (5 g) and the mixture was stirred at ambient temperature for 30 min. The insoluble material was filtered and the filtrate was concentrated under reduced pressure. The residue was dissolved in CHCl₃ (100 mL) and the insoluble material was filtered. The filtrate was concentrated under reduced pressure to give 5,6-dimethylpyridin-2(1B)-one (2.19 g) as a yellow solid.

¹H NMR (300 MHz, CDCl₃, δ): 2.05 (s, 3H), 2.31 (s, 3H), 6.38 (d, J=9.2 Hz, 1H), 7.26 (d, J=9.2 Hz, 1H); ESI MS m/z 124 (M⁺+1, 100%).

### Step D: Synthesis of 6-chloro-2,3-dimethylpyridine

A suspension of 5,6-dimethylpyridin-2(1H)-one (1.34 g) and N,N-dimethylaniline (1.53 mL) in POCl₃ (2.12 mL) was stirred at reflux for 40 min. After cooling, the mixture was poured into ice water (50 mL) below 10° C. The aqueous layer was extracted with CHCl₃ three times. The combined organic layer was dried over MgSO₄, filtrated, concentrated under reduced pressure, and purified by medium-pressure liquid chromatography (silica gel, 6% to 20% EtOAc in hexane) to give 6-chloro-2,3-dimethylpyridine (740 mg) as a colorless oil.

¹H NMR (300 MHz, CDCl₃, δ): 2.25 (s, 3H), 2.47 (s, 3H), 7.06 (d, J=7.9 Hz, 1H), 7.36 (d, J=7.6 Hz, 1H); ESI MS m/z 142 (M⁺+1, 100%).

### Step E: Synthesis of 3-chloro-N-{cis-4-[(5,6-dimethylpyridin-2-yl)amino]cyclohexyl}-4-fluorobenzamide hydrochloride

A mixture of 6-chloro-2,3-dimethylpyridine (400 mg), N-(cis-4-aminocyclohexyl)-3-chloro-4-fluorobenzamide obtained in step A of example 1 (841 mg), and BuOH (0.8 mL) was heated in a microwave synthesizer at 180° C. for 20 min and 230° C. for 50 min. The mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 20% to 99% EtOAc in hexane and silica gel, 3% to 5% MeOH in CHCl₃) to give 3-chloro-N-{cis-4-[(5,6-dimethylpyridin-2-yl)amino]cyclohexyl}-4-fluorobenzamide. To a solution of the above material in EtOAc (3 mL) was added 4 M hydrogen chloride in EtOAc (0.18 mL). The mixture was stirred at ambient temperature for 4 h and concentrated under reduced pressure. The residue was suspended in Et₂O and the suspension was stirred at ambient temperature. The precipitate was collected by filtration, washed with Et₂O, and dried at 80° C. under reduced pressure to give 3-chloro-N-{cis-4-[(5,6-dimethylpyridin-2-yl)amino]cyclohexyl}-4-fluorobenzamide hydrochloride (112 mg) as a colorless powder.

¹H NMR (300 MHz, CDCl₃, δ): 1.70-2.01 (m, 8H), 2.19 (s, 3H), 2.53 (s, 3H), 3.74-3.84 (m, 1H), 4.04-4.20 (m, 1H), 6.56 (d, J=9.0 Hz, 1H), 6.63 (d, J=8.9 Hz, 1H), 7.18 (t, J=8.6 Hz, 1H), 7.59 (d, J=8.9 Hz, 1H), 7.67-7.74 (m, 1H), 7.94 (dd, J=7.1, 2.3 Hz, 1H), 8.71 (d, J=8.6 Hz, 1H), 14.74 (brs, 1H); ESI MS m/z 376 [M (free)⁺+1, 100%].

### Example 44

### 3-Chloro-4-fluoro-N-{cis-4-[(4-methoxypyridin-2-yl)amino]cyclohexyl}benzamide hydrochloride

Using the procedure for the step B of example 1, the title compound was obtained.

¹H NMR (300 MHz, CDCl₃, δ): 1.63-2.03 (m, 8H), 3.73-3.82 (m, 1H), 3.96 (s, 3H), 4.05-4.21 (m, 1H), 6.00 (d, J=2.3 Hz, 1H), 6.33 (dd, J=7.2, 2.4 Hz, 1H), 6.65 (d, J=8.6 Hz, 1H), 7.19 (t, J=8.6 Hz, 1H), 7.58 (d, J=7.3 Hz, 1H), 7.68-7.75 (m, 1H), 7.94 (dd, J=7.0, 2.2 Hz, 1H), 9.01 (d, J=7.9 Hz, 1H); ESI MS m/z 378 [M (free)⁺+1, 100%].

### Example 45

### 3-Chloro-N-{cis-4-[(4-cyanopyridin-2-yl)amino]cyclohexyl}-4-fluorobenzamide hydrochloride

Using the procedure for the step B of example 1, the title compound was obtained.

¹H NMR (300 MHz, CDCl₃, δ): 1.71-2.04 (m, 8H), 3.81-3.91 (m, 1H), 4.08-4.21 (m, 1H), 6.23 (d, J=7.8 Hz, 1H), 6.78-6.85 (m, 2H), 7.12 (brs, 1H), 7.20 (t, J=8.6 Hz, 1H), 7.64-7.71 (m, 1H), 7.87 (dd, J=7.0, 2.2 Hz, 1H), 8.07 (d, J=5.6 Hz, 1H); ESI MS m/z 373 [M (free)⁺+1, 100%].

### Example 46

### 2-({cis-4-[(3-Chloro-4-fluorobenzoyl)amino]cyclohexyl}amino)isonicotinamide

### Step A: Synthesis of 2-chloroisonicotinamide

To a solution of 2-chloroisonicotinic acid (2.00 g) in DMF (20 mL) were added aqueous 28% NH₃ (0.93 mL), Et₃N (4.42 mL), EDC-HCl (2.92 g), and HOBt-H₂O (2.92 g). The mixture was stirred at ambient temperature for 17 h and added to H₂O (100 mL). The mixture was stirred at ambient temperature for 2 h. The precipitate was collected by filtration, washed with H₂O, and dried under reduced pressure to give 2-chloroisonicotinamide (934 mg) as a colorless solid.

¹H NMR (300 MHz, DMSO-d₆, δ): 7.78 (dd, J=5.1, 1.5 Hz, 1H), 7.83-7.92 (m, 2H), 8.32 (brs, 1H), 8.56 (dd, J=5.1, 0.8 Hz, 1H); ESI MS m/z 155 (M⁺−1, 100%), 157 (M⁺+1, 10%).

### Step B: Synthesis of 2-({cis-4-[(3-chloro-4-fluorobenzoyl)amino]cyclohexyl}amino) isonicotinamide

A mixture of 2-chloroisonicotinamide (400 mg) and N-(cis-4-aminocyclohexyl)-3-chloro-4-fluorobenzamide obtained in step A of example 1 (761 mg) in BuOH (2 mL) was heated in a microwave synthesizer at 180° C. for 20 min and 200° C. for 80 min. The mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 33% to 99% EtOAc in hexane and flash silica gel, 3% to 10% MeOH in CHCl₃) to give 2-({cis-4-[(3-chloro-4-fluorobenzoyl)amino]cyclohexyl}-amino) isonicotinamide (45 mg) as a colorless solid.

¹H NMR (300 MHz, DMSO-d₆, δ): 1.59-1.90 (m, 8H), 3.85 (brs, 2H), 6.72 (brs, 1H), 6.84 (dd, J=5.4, 1.2 Hz, 1H), 6.99 (brs, 1H), 7.47-7.56 (m, 2H), 7.86-7.93 (m, 1H), 7.97 (brs, 1H), 8.03 (d, J=5.3 Hz, 1H), 8.10 (dd, J=7.2, 2.2 Hz, 1H), 8.32 (d, J=6.4 Hz, 1H); ESI MS m/z 390 [M (free)⁺+1, 100%].

### Example 47

### 2-({cis-4-[(3-Chloro-4-fluorobenzoyl)amino]cyclohexyl}amino)-N,N-dimethylisonicotinamide hydrochloride

### Step A: Synthesis of 2-chloro-N,N-dimethylisonicotinamide

To a solution of 2-chloroisonicotinic acid (2.00 g) in DMF (20 mL) were added aqueous 50% Me₂NH (1.37 mL), Et₃N (4.42 mL), EDC-HCl (2.92 g), and HOBt-H₂O (2.92 g). The mixture was stirred at ambient temperature for 17 h and added to H₂O (100 mL). The aqueous layer was extracted with CHCl₃ three times. The combined organic layer was dried over MgSO₄, filtrated, and concentrated under reduced pressure. The residue was diluted with EtOAc and the solution was washed with H₂O and brine two times. The aqueous layer was extracted with mixture of EtOAc and CHCl₃ five times. The combined organic layer was dried over MgSO₄, filtrated, concentrated under reduced pressure, and purified by medium-pressure liquid chromatography (silica gel, 20% to 99% EtOAc in hexane) to give 2-chloro-N,N-dimethylisonicotinamide (2.04 g) as a pale yellow oil.

¹H NMR (300 MHz, CDCl₃, δ): 2.96 (s, 3H), 3.12 (s, 3H), 7.23 (dd, J=5.1, 1.3 Hz, 1H), 7.34-7.35 (m, 1H), 8.47 (dd, J=5.0, 0.8 Hz, 1H); ESI MS m/z 185 (M⁺+1, 45%), 207 (M⁺+23, 100%).

### Step B: Synthesis of 2-({cis-4-[(3-chloro-4-fluorobenzoyl)amino]cyclohexyl}amino)-N,N-dimethylisonicotinamide hydrochloride

A mixture of 2-chloro-N,N-dimethylisonicotinamide (400 mg) and N-(cis-4-aminocyclohexyl)-3-chloro-4-fluorobenzamide obtained in step A of example 1 (645 mg) in BuOH (2 mL) was heated in a microwave synthesizer at 180° C. for 20 min, 200° C. for 20 min, and 220° C. for 80 min. The mixture was diluted with CHCl₃ and added to aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 33% to 99% EtOAc in hexane and silica gel, 3% to 10% MeOH in CHCl₃) to give 2-({cis-4-[(3-chloro-4-fluorobenzoyl)amino]cyclohexyl}amino)-N,N-dimethylisonicotinamide. To a solution of the above material in EtOAc (4 mL) was added 4 M hydrogen chloride in EtOAc (0.16 mL). The mixture was stirred at ambient temperature for 4 h and concentrated under reduced pressure. The residue was suspended in Et₂O and the suspension was stirred at ambient temperature for 4 h. The precipitate was collected by filtration, washed with Et₂O, and dried at 80° C. under reduced pressure to give 2-({cis-4-[(3-chloro-4-fluorobenzoyl)amino]cyclohexyl}amino)-N,N-dimethylisonicotinamide hydrochloride (116 mg) as a pale yellow powder.

¹H NMR (300 MHz, CDCl₃, δ): 1.76-2.03 (m, 8H), 3.00 (s, 3H), 3.13 (s, 3H), 3.80-3.89 (m, 1H), 4.06-4.20 (m, 1H), 6.61-6.70 (m, 2H), 6.80 (brs, 1H), 7.19 (t, J=8.6 Hz, 1H), 7.68-7.75 (m, 1H), 7.81 (d, J=6.4 Hz, 1H), 7.95 (dd, J=7.1, 2.3 Hz, 1H), 9.17-9.26 (m, 1H); ESI MS m/z 419 [M (free)⁺+1, 80%], 441 [M (free)⁺+23, 100%].

### Example 48

### 3-Chloro-4-fluoro-N-(cis-4-{[4-(hydroxymethyl)pyridin-2-yl]amino}cyclohexyl)benzamide hydrochloride

### Step A: Synthesis of methyl 2-chloroisonicotinate

To a solution of 2-chloroisonicotinic acid (6.00 g) in DMF (0.29 mL) and CHCl₃ (60 mL) cooled on an ice-bath was added thionyl chloride (3.2 mL) and the mixture was stirred at reflux for 1 h. To the mixture was added thionyl chloride (1.6 mL) and the mixture was stirred at reflux for 30 min. To the mixture was added thionyl chloride (3.2 mL) and the mixture was stirred at reflux for 30 min and concentrated under reduced pressure. The residue was diluted with CHCl₃ (20 mL) and the solution was poured into a mixture of MeOH (1.85 mL), CHCl₃ (19 mL), and Et₃N (6.4 mL) cooled on an ice-bath. The mixture was stirred at 0° C. for 5 min and at ambient temperature for 60.5 h and quenched with aqueous saturated NaHCO₃. The aqueous layer was extracted with CHCl₃ three times. The combined organic layer was dried over MgSO₄, filtrated, concentrated under reduced pressure, and purified by medium-pressure liquid chromatography (silica gel, 9% to 20% EtOAc in hexane) to give methyl 2-chloroisonicotinate (6.05 g) as a colorless oil.

¹H NMR (300 MHz, CDCl₃, δ): 3.95-4.01 (m, 3H), 7.76-7.81 (m, 1H), 7.88-7.93 (m, 1H), 8.53-8.59 (m, 1H); CI MS m/z 172 (M⁺+1, 100%).

### Step B: Synthesis of (2-chloropyridin-4-yl)methanol

To a suspension of lithium aluminumhydride (221 mg) in Et₂O (10.0 mL) cooled to −4° C. was added methyl 2-chloroisonicotinate (1.00 g) in Et₂O (2 mL) dropwise and the mixture was stirred at −4° C. for 30 min and quenched with water. The aqueous layer was extracted with EtOAc three times. The combined organic layer was washed with aqueous saturated NaCl, dried over MgSO₄, filtered, and concentrated under reduced pressure to give (2-chloropyridin-4-yl)methanol (641 mg) as a pale brown solid.

¹H NMR (300 MHz, CDCl₃, δ): 2.81-2.93 (m, 1H), 4.72-4.80 (m, 2H), 7.17-7.26 (m, 1H), 7.34-7.41 (m, 1H), 8.30 (dd, J=5.1, 0.6 Hz, 1H); ESI MS m/z 144 (M⁺+1, 100%).

### Step C: Synthesis of 3-chloro-4-fluoro-N-(cis-4-{[4-(hydroxymethyl)pyridin-2-yl]amino}cyclohexyl)benzamide hydrochloride

A mixture of (2-chloropyridin-4-yl)methanol (250 mg), N-(cis-4-aminocyclohexyl)-3-chloro-4-fluorobenzamide obtained in step A of example 1 (566 mg), and BuOH (1 mL) was heated in a microwave synthesizer at 220° C. for 20 min and 240° C. for 20 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 33% to 100% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-(cis-4-{[4-(hydroxymethyl)pyridin-2-yl]amino}cyclohexyl)benzamide hydrochloride (68.0 mg) as a white solid.

¹H NMR (300 MHz, DMSO-d₆, δ): 1.61-1.98 (m, 8H), 3.80-3.98 (m, 2H), 4.53 (s, 2H), 6.74 (d, J=7.0 Hz, 1H), 7.12 (s, 1H), 7.53 (t, J=8.9 Hz, 1H), 7.81-7.95 (m, 2H), 8.11 (dd, J=7.4, 1.9 Hz, 1H), 8.41 (d, J=6.4 Hz, 1H), 8.64-8.76 (m, 1H); ESI MS m/z 378 [M (free)⁺+1, 100%].

### Example 49

### 3-Chloro-4-fluoro-N-[cis-4-({5-methyl-4-[methyl(2-phenylethyl)amino]pyridin-2-yl}amino)cyclohexyl]benzamide hydrochloride

### Step A: Synthesis of N²-[cis-4-(benzylamino)cyclohexyl]-N⁴,5-dimethyl-N⁴-(2-phenylethyl)pyridine-2,4-diamine

A mixture of cis-N-benzyl-N′-(4-chloro-5-methyl-1-oxidopyridin-2-yl)cyclohexane-1,4-diamine obtained in step D of example 4 (1.00 g), N-methyl-2-phenylethanamine (469 mg), and BuOH (1 mL) was heated in a microwave synthesizer at 220° C. for 20 min. The mixture was diluted with CHCl₃ and poured into saturated aqueous NaHCO₃. The aqueous layer was extracted with CHCl₃ three times. The combined organic layer was dried over MgSO₄, filtrated, concentrated under reduced pressure, and purified by medium-pressure liquid chromatography (NH-silica gel, 20% to 50% EtOAc in hexane) to give N²-[cis-4-(benzylamino)cyclohexyl]-N⁴,5-dimethyl-N-(2-phenylethyl)pyridine-2,4-diamine (269 mg) as a pale brown oil.

¹H NMR (300 MHz, CDCl₃, δ): 1.42-1.87 (m, 8H), 2.10 (s, 3H), 2.63-2.75 (m, 1H), 2.78-2.89 (m, 5H) 3.19-3.30 (m, 2H), 3.74-3.84 (m, 3H), 4.31-4.41 (m, 1H), 5.86 (s, 1H), 7.12-7.39 (m, 10H), 7.73 (s, 1H); ESI MS m/z 429 (M⁺, 100%).

### Step B: Synthesis of N²-(cis-4-aminocyclohexyl)-N⁴,5-dimethyl-N⁴-(2-phenylethyl)pyridine-2,4-diamine

A mixture of N²-[cis-4-(benzylamino)cyclohexyl]-N⁴,5-dimethyl-N⁴-(2-phenylethyl)pyridine-2,4-diamine (260 mg), 10% palladium carbon (52.0 mg), and MeOH (3 mL) was stirred at 50° C. under hydrogen atmosphere for 60 h. The mixture was filtered through a pad of celite, concentrated under reduced pressure, and purified by medium-pressure liquid chromatography (NH-silica gel, 50% EtOAc in hexane to 2% MeOH in CHCl₃) to give N²-(cis-4-aminocyclohexyl)-N⁴,5-dimethyl-N⁴-(2-phenylethyl)pyridine-2,4-diamine (154 mg) as a pale brown oil.

¹H NMR (300 MHz, CDCl₃, δ): 1.34-1.91 (m, 8H), 2.10 (s, 3H), 2.74-2.91 (m, 6H), 3.20-3.30 (m, 2H), 3.69-3.82 (m, 1H), 4.33-4.43 (m, 1H), 5.87 (s, 1H), 7.11-7.33 (m, 5H), 7.73 (s, 1H); ESI MS m/z 339 (M⁺+1, 100%).

### Step C: Synthesis of 3-chloro-4-fluoro-N-[cis-4-({5-methyl-4-[methyl(2-phenylethyl)amino]pyridin-2-yl}amino)cyclohexyl]benzamide hydrochloride

To a solution of N²-(cis-4-aminocyclohexyl)-N⁴,5-dimethyl-N⁴-(2-phenylethyl)pyridine-2,4-diamine (150 mg) in DMF (2.00 mL) were added 3-chloro-4-fluorobenzoic acid (93.0 mg), Et₃N (148 μL), HOBt-H₂O (101 mg), and EDC-HCl (102 mg). The mixture was stirred at ambient temperature for 14 h and poured into saturated aqueous NaHCO₃. The aqueous layer was extracted with CHCl₃ three times. The combined organic layer was dried over MgSO₄, filtered, concentrated under reduced pressure, and purified by medium-pressure liquid chromatography (NH-silica gel, 20% EtOAc in hexane). To a solution of the above purified material in EtOAc (10 mL) was added 4 M hydrogen chloride in EtOAc (0.5 mL). The mixture was stirred at ambient temperature for 1 h and concentrated under reduced pressure. A suspension of the residue in Et₂O (10 mL) was stirred at ambient temperature for 4 h. The precipitate was collected by filtration, washed with Et₂O, and dried at 80° C. under reduced pressure to give 3-chloro-4-fluoro-N-[cis-4-({5-methyl-4-[methyl(2-phenylethyl)amino]pyridin-2-yl}amino)cyclohexyl]benzamide hydrochloride (22.0 mg) as a white solid.

¹H NMR (300 MHz, CDCl₃, δ): 1.64-1.99 (m, 8H), 2.18 (s, 3H), 2.89-2.96 (m, 2H), 3.02 (s, 3H), 3.54-3.71 (m, 3H), 4.05-4.19 (m, 1H), 5.62 (s, 1H), 6.77-6.86 (m, 1H), 7.10-7.34 (m, 7H), 7.67-7.78 (m, 1H), 7.96 (dd, J=7.1, 2.1 Hz, 1H), 8.23-8.31 (m, 1H), 13.25-13.32 (m, 1H); ESI MS m/z 495 [M (free)⁺+1, 100%].

### Example 50

### 3-Chloro-4-fluoro-N-{cis-4-[(4,5,6-trimethylpyridin-2-yl)amino]cyclohexyl}benzamide hydrochloride

### Step A: Synthesis of 4,5,6-trimethylpyridin-2(1H)-one

A mixture of 3-oxobutanamide (10.0 g), butan-2-one (14.7 g) and polyphospholic acid (70.6 g) was stirred at reflux for 4 h and poured into ice water (500 mL). The mixture was stirred for 1 h, neutralized with saturated aqueous NaHCO₃ (pH=7) and stirred for 11 h. The precipitate was collected by filtration, washed with water and hexane and dried at 80° C. under reduced pressure to give the title compound (8.99 g).

¹H NMR (300 MHz, CDCl₃, δ): 1.96 (s, 3H), 2.16 (s, 3H), 2.32 (s, 3H), 6.28 (s, 1H); ESI MS m/z 160 (M⁺+23, 100%), 138 (M⁺+1, 40%).

### Step B: Synthesis of 6-chloro-2,3,4-trimethylpyridine

A mixture of 4,5,6-trimethylpyridin-2(1H)-one (8.50 g), N,N-dimethylaniline (8.69 mL) and POCl₃ (12.1 mL) was stirred at reflux for 40 min and poured into ice water (100 mL). The mixture was stirred for 30 min and the aqueous layer was extracted with CHCl₃ (three times). The combined organic layer was dried over MgSO₄, filtered, concentrated under reduced pressure and purified by medium-pressure liquid chromatography (silica gel, 6% to 9% EtOAc in hexane) to give the title compound (4.46 g).

¹H NMR (300 MHz, CDCl₃, δ): 2.16 (s, 3H), 2.26 (s, 3H), 2.48 (s, 3H), 6.97 (s, 1H); ESI MS m/z 156 (M⁺+1, 100%).

### Step C: Synthesis of 3-chloro-4-fluoro-N-{cis-4-[(4,5,6-trimethylpyridin-2-yl)amino]-cyclohexyl}benzamide hydrochloride

The title compound (345 mg) was prepared from N-(cis-4-aminocyclohexyl)-3-chloro-4-fluorobenzamide obtained in step A of example 1 (1.15 g) and 6-chloro-2,3,4-trimethylpyridine (600 mg) using the procedure for the step B of example 1.

¹H NMR (300 MHz, CDCl₃, δ): 1.67-2.01 (m, 8H), 2.09 (s, 3H), 2.34 (s, 3H), 2.52 (s, 3H), 3.72-3.82 (m, 1H), 4.05-4.19 (m, 1H), 6.42 (s, 1H), 6.61-6.71 (m, 1H), 7.18 (t, J=8.6 Hz, 1H), 7.71 (ddd, J=8.6, 4.5, 2.3 Hz, 1H), 7.94 (dd, J=7.0, 2.2 Hz, 1H), 8.42-8.53 (m, 1H); ESI MS m/z 390 [M (free)⁺+1, 100%].

### Example 51

### 3-Chloro-N-{cis-4-[(4,5-dimethylpyridin-2-yl)amino]cyclohexyl}-4-fluorobenzamide hydrochloride

### Step A: Synthesis of 3,4-dimethylpyridine 1-oxide

To a solution of 3,4-dimethylpyridine (10.0 g) in CHCl₃ (100 mL) was added a suspension of m-chloroperbenzoic acid (24.8 g) in CHCl₃ (100 mL) at 0° C. and the mixture was stirred at 0° C. for 1.5 h. To the mixture was added saturated aqueous Na₂S₂O₃ and the organic layer was separated. The aqueous layer was extracted with CHCl₃ (three times). The combined organic layer was washed with 1M aqueous NaOH and saturated aqueous NaCl, dried over MgSO₄, filtered, concentrated under reduced pressure and purified by medium-pressure liquid chromatography (NH-silica gel, 50% EtOAc in hexane to 3% MeOH in CHCl₃) to give the title compound (4.94 g).

¹H NMR (300 MHz, CDCl₃, δ): 2.22 (s, 3H), 2.26 (s, 3H), 7.02 (d, J=6.2 Hz, 1H), 7.94-8.06 (m, 2H); ESI MS m/z 124 (M⁺+1, 100%).

### Step B: Synthesis of 2-chloro-4,5-dimethylpyridine

To a solution of 3,4-dimethylpyridine 1-oxide (4.50 g) in CHCl₃ (45 mL) was added a solution of POCl₃ (4.43 mL) in CHCl₃ (26.5 mL) under 6° C. and the mixture was stirred at ambient temperature for 21.5 h. The mixture was poured into ice water (200 mL) and the organic layer was separated. The aqueous layer was extracted with CHCl₃ (three times). The combined organic layer was dried over MgSO₄, filtered, concentrated under reduced pressure and purified by medium-pressure liquid chromatography (silica gel, 9% EtOAc in hexane) to give the title compound (277 mg).

¹H NMR (300 MHz, CDCl₃, δ): 2.21 (s, 3H), 2.26 (s, 3H), 7.10 (s, 1H), 8.09 (s, 1H); ESI MS m/z 142 (M⁺+1, 100%).

### Step C: Synthesis of 3-chloro-N-{cis-4-[(4,5-dimethylpyridin-2-yl)amino]cyclohexyl}-4-fluorobenzamide hydrochloride

The title compound (92.0 mg) was prepared from N-(cis-4-aminocyclohexyl)-3-chloro-4-fluorobenzamide obtained in step A of example 1 (526 mg) and 2-chloro-4,5-dimethylpyridine (250 mg) using the procedure for the step B of example 1.

¹H NMR (300 MHz, CDCl₃, δ): 1.74-2.03 (m, 8H), 2.16 (s, 3H), 2.36 (s, 3H), 3.75-3.85 (m, 1H), 4.07-4.20 (m, 1H), 6.60 (s, 1H), 6.64-6.71 (m, 1H), 7.18 (t, J=8.6 Hz, 1H), 7.45 (s, 1H), 7.71 (ddd, J=8.6, 4.5, 2.3 Hz, 1H), 7.95 (dd, J=7.0, 2.2 Hz, 1H), 8.76-8.83 (m, 1H); ESI MS m/z 376 [M (free)⁺+1, 100%].

### Example 52

### 3-Chloro-N-{cis-4-[(4,6-dimethylpyridin-2-yl)amino]cyclohexyl}-4-fluorobenzamide hydrochloride

### Step A: Synthesis of 2,4-dimethylpyridine 1-oxide

The title compound (8.17 g) was prepared from 2,4-dimethylpyridine (10.0 g) using the procedure for the step A of example 51.

¹H NMR (300 MHz, CDCl₃, δ): 2.32 (s, 3H), 2.50 (s, 3H), 6.92-6.97 (m, 1H), 7.05-7.09 (m, 1H), 8.15 (d, J=6.7 Hz, 1H); ESI MS m/z 146 (M⁺+23, 100%), 124 (M⁺+1, 80%).

### Step B: Synthesis of 2-chloro-4,6-dimethylpyridine

The title compound (557 mg) was prepared from 2,4-dimethylpyridine 1-oxide (8.00 g) using the procedure for the step B of example 51.

¹H NMR (300 MHz, CDCl₃, δ): 2.30 (s, 3H), 2.48 (s, 3H), 6.89 (s, 1H), 6.97 (s, 1H); ESI MS m/z 142 (M⁺+1, 100%).

### Step C: Synthesis of 3-chloro-N-{cis-4-[(4,6-dimethylpyridin-2-yl)amino]cyclohexyl}-4-fluorobenzamide hydrochloride

The title compound (274 mg) was prepared from N-(cis-4-aminocyclohexyl)-3-chloro-4-fluorobenzamide obtained in step A of example 1 (946 mg) and 2-chloro-4,6-dimethylpyridine (450 mg) using the procedure for the step B of example 1.

¹H NMR (300 MHz, CDCl₃, δ): 1.73-2.03 (m, 8H), 2.37 (s, 3H), 2.51 (s, 3H), 3.75-3.84 (m, 1H), 4.07-4.20 (m, 1H), 6.34 (s, 1H), 6.37 (s, 1H), 6.70-6.77 (m, 1H), 7.18 (t, J=8.5 Hz, 1H), 7.72 (ddd, J=8.5, 4.5, 2.3 Hz, 1H), 7.95 (dd, J=7.1, 2.3 Hz, 1H), 8.71-8.82 (m, 1H); ESI MS m/z 376 [M (free)⁺+1, 100%].

### Example 53

### 3-Chloro-4-fluoro-N-{cis-4-[(3,5,6-trimethylpyridin-2-yl)amino]cyclohexyl}benzamide hydrochloride

### Step A: Synthesis of 2,3,5-trimethylpyridine 1-oxide.

The title compound (11.8 g) was prepared from 2,3,5-trimethylpyridine (10.0 g) using the procedure for the step A of example 51.

¹H NMR (300 MHz, CDCl₃, δ): 2.23 (s, 3H), 2.30 (s, 3H), 2.46 (s, 3H), 6.89 (s, 1H), 8.03 (s, 1H); ESI MS m/z 160 (M⁺+23, 100%), 138 (M⁺+1, 90%).

### Step B: Synthesis of 2-chloro-3,5,6-trimethylpyridine

The title compound (937 mg) was prepared from 2,3,5-trimethylpyridine 1-oxide (11.5 g) using the procedure for the step B of example 51.

¹H NMR (300 MHz, CDCl₃, δ): 2.31 (s, 3H), 2.35 (s, 3H), 2.52 (s, 3H), 8.15 (s, 1H); ESI MS m/z 156 (M⁺+1, 100%).

### Step C: Synthesis of 3-chloro-4-fluoro-N-{cis-4-[(3,5,6-trimethylpyridin-2-yl)amino]-cyclohexyl}benzamide hydrochloride

The title compound (10 mg) was prepared from N-(cis-4-aminocyclohexyl)-3-chloro-4-fluorobenzamide obtained in step A of example 1 (905 mg) and 2-chloro-3,5,6-trimethylpyridine (400 mg) using the procedure for the step B of example 1.

¹H NMR (300 MHz, CDCl₃, δ): 1.73-2.24 (m, 11H), 2.34 (s, 3H), 2.62 (s, 3H), 4.02-4.35 (m, 1H), 4.38-4.60 (m, 1H), 6.82-7.27 (m, 2H), 7.30-7.56 (m, 2H), 7.80-8.15 (m, 2H); ESI MS m/z 390 [M (free)⁺+1, 100%].

### Example 54

### 3-Chloro-4-fluoro-N-{cis-4-[(3-fluoro-4-methylpyridin-2-yl)amino]cyclohexyl}benzamide hydrochloride

### Step A: Synthesis of 3-fluoro-4-methylpyridine 1-oxide

To a solution of diisopropylamine in THF (200 mL) was added a 2.44 M n-BuLi in THF (116 mL) at 0° C. and the mixture was stirred at 0° C. for 20 min. The mixture was cooled to −60° C. and a solution of 3-fluoropyridine (25.0 g) in THF (100 mL) was added. The mixture was stirred at −60° C. for 3 h and a solution of iodomethane (17.6 mL) in THF (100 mL) was added. The mixture was stirred at −60° C. for 30 min and the reaction was quenched with saturated aqueous NH₄Cl (100 mL). The aqueous layer was extracted with EtOAc (three times). The combined organic layer was dried over MgSO₄, filtered, concentrated under reduced pressure to give a colorless oil (14.6 g). To a solution of the above oil (14.6 g) in CHCl₃ (145 mL) was added a suspension of m-chloroperbenzoic acid (34.8 g) in CHCl₃ (145 mL) at 0° C. and the mixture was stirred at ambient temperature for 3 h. To the mixture was added saturated aqueous Na₂S₂O₃ and the organic layer was separated. The aqueous layer was extracted with CHCl₃ (three times). The combined organic layer was washed with 1M aqueous NaOH and saturated aqueous NaCl, dried over MgSO₄, filtered, concentrated under reduced pressure and purified by medium-pressure liquid chromatography (silica gel, 2% to 4% MeOH in CHCl₃) to give the title compound (3.47 g).

¹H NMR (300 MHz, CDCl₃, δ): 2.28-2.32 (m, 3H), 7.06-7.13 (m, 1H), 7.96-8.00 (m, 1H), 8.11 (dd, J=4.9, 1.8 Hz, 1H); ESI MS m/z 150 (M⁺+23, 100%).

### Step B: Synthesis of 2-chloro-3-fluoro-4-methylpyridine

The title compound (960 mg) and 2-chloro-5-fluoro-4-methylpyridine (1.24 g) were prepared from 3-fluoro-4-methylpyridine 1-oxide (3.1 g) using the procedure for the step B of example 51.

### 2-chloro-3-fluoro-4-methylpyridine

¹H NMR (300 MHz, CDCl₃, δ): 2.31-2.41 (m, 3H), 7.06-7.17 (m, 1H), 8.06 (d, J=4.8 Hz, 1H); ESI MS m/z 146 (M⁺+1, 30%).

### 2-chloro-5-fluoro-4-methylpyridine

¹H NMR (300 MHz, CDCl₃, δ): 2.29-2.33 (m, 3H), 7.17-7.21 (m, 1H), 8.12-8.15 (m, 1H); ESI MS m/z 146 (M⁺+1, 10%).

### Step C: Synthesis of 3-chloro-4-fluoro-N-{cis-4-[(3-fluoro-4-methylpyridin-2-yl)amino]-cyclohexyl}benzamide hydrochloride

The title compound (106 mg) was prepared from N-(cis-4-aminocyclohexyl)-3-chloro-4-fluorobenzamide obtained in step A of example 1 (615 mg) and 2-chloro-3-fluoro-4-methylpyridine (300 mg) using the procedure for the step B of example 1.

¹H NMR (300 MHz, CDCl₃, δ): 1.78-2.07 (m, 8H), 2.28-2.37 (m, 3H), 4.08-4.23 (m, 1H), 4.26-4.40 (m, 1H), 6.31-6.44 (m, 1H), 6.50-6.59 (m, 1H), 7.19 (t, J=8.5 Hz, 1H), 7.60-7.74 (m, 2H), 7.86-7.94 (m, 1H); ESI MS m/z 380 [M (free)⁺+1, 100%].

### Example 55

### 3-Chloro-4-fluoro-N-{cis-4-[(5-fluoro-4-methylpyridin-2-yl)amino]cyclohexyl}benzamide hydrochloride

The title compound (6 mg) was prepared from N-(cis-4-aminocyclohexyl)-3-chloro-4-fluorobenzamide obtained in step A of example 1 (615 mg) and 2-chloro-5-fluoro-4-methylpyridine (300 mg) obtained in step B of example 54 using the procedure for the step B of example 1.

¹H NMR (300 MHz, CDCl₃, δ): 0.73-0.97 (m, 2H), 1.69-2.07 (m, 6H), 2.43 (s, 3H), 3.73-3.85 (m, 1H), 4.02-4.20 (m, 1H), 6.55-6.74 (m, 2H), 7.18 (t, J=8.5 Hz, 1H), 7.54-7.77 (m, 2H), 7.94 (d, J=6.8 Hz, 1H), 8.70-8.89 (m, 1H); ESI MS m/z 380 [M (free)⁺+1, 100%].

### Example 56

### 4-Fluoro-N-{cis-3-[(6-methylpyridin-2-yl)amino]cyclopentyl}benzamide

### Step A: Synthesis of tert-butyl 3-oxo-2-azabicyclo[2.2.1]hept-5-ene-2-carboxylate.

To a solution of 2-azabicyclo[2.2.1]hept-5-en-3-one (40.2 g) in pyridine (100 mL) were added N,N-dimethylpyridin-4-amine (47.3 g) and a solution of (Boc)₂O (96.5 g) in pyridine (40.0 mL). The mixture was stirred at ambient temperature for 15 h and pyridine was evaporated under reduced pressure. To the residue were added EtOAc (500 mL) and H₂O (500 mL) and the organic layer was separated. The aqueous layer was extracted with EtOAc (twice). The combined organic layer was washed with H₂O and saturated aqueous NaCl, dried over MgSO₄, filtered, concentrated under reduced pressure and purified by flush chromatography (silica gel, 25% EtOAc in hexane) to give the title compound (69.6 g).

¹H NMR (200 MHz, CDCl₃, δ): 1.44-1.60 (m, 9H), 2.10-2.23 (m, 1H), 2.28-2.43 (m, 1H), 3.34-3.45 (m, 1H), 4.92-5.03 (m, 1H), 6.60-6.74 (m, 1H), 6.84-6.97 (m, 1H); ESI MS m/z 210 (M⁺+1, 50%).

### Step B: Synthesis of methyl cis-3-[(tert-butoxycarbonyl)amino]cyclopentanecarboxylate

To a solution of tert-butyl 3-oxo-2-azabicyclo[2.2.1]hept-5-ene-2-carboxylate (69.3 g) in MeOH (900 mL) was added 5% Pd—C (11.5 g) and the mixture was stirred at ambient temperature for 4 h under hydrogen atmosphere. The mixture was filtered through a pad of celite, concentrated under reduced pressure, and purified by flush chromatography (silica gel, 25% EtOAc in hexane) to give a white solid (69.6 g). To the solution of above solid in MeOH (300 mL) was added sodium methoxide (3.53 g). The mixture was stirred at ambient temperature for 3 h and evaporated under reduced pressure. To the residue were added EtOAc and 5% KHSO₄ and the organic layer was separated. The organic layer was washed saturated aqueous NaCl, dried over MgSO₄, filtered, concentrated under reduced pressure and purified by flush chromatography (silica gel, 20% EtOAc in hexane) to give the title compound (71.2 g).

¹H NMR (200 MHz, CDCl₃, δ): 1.44 (s, 9H), 1.54-2.03 (m, 4H), 2.11-2.33 (m, 1H), 2.71-2.96 (m, 1H), 3.69 (s, 3H), 3.94-4.23 (m, 2H), 4.86-5.09 (m, 1H); ESI MS m/z 266 (M⁺+23, 100%).

### Step C: Synthesis of benzyl tert-butyl cis-cyclopentane-1,3-diylbiscarbamate

To a solution of methyl cis-3-[(tert-butoxycarbonyl)amino]cyclopentanecarboxylate (70.9 g) in MeOH (300 mL) was added aqueous NaOH (NaOH 12.2 g, H₂O 15.0 mL). The mixture was stirred at ambient temperature for 16 h and the mixture was evaporated under reduced pressure. To the residue was added 5% KHSO₄ (250 mL). The precipitate was collected by filtration and dried at 80° C. under reduced pressure to give a white solid (87.7 g). To the suspension of above solid (66.8 g) in toluene (500 mL) were added diphenylphosphoryl azide (84.2 g) in toluene (50.0 mL) and Et₃N (35.4 g) in toluene (50.0 mL) and the mixture was stirred at 100° C. for 35 min. To the mixture was added benzyl alcohol (33.1 g). The mixture was stirred at 130° C. for 2 h and evaporated under reduced pressure. To the residue were added EtOAc (800 mL) and H₂O (600 mL) and the organic layer was separated. The organic layer was dried over MgSO₄, filtered, concentrated under reduced pressure and purified by flush chromatography (silica gel, 13% to 25% EtOAc in hexane) to give the title compound (43.8 g).

¹H NMR (200 MHz, CDCl₃, δ): 1.26-1.71 (m, 12H), 1.81-2.08 (m, 2H), 2.30-2.52 (m, 1H), 3.82-4.09 (m, 2H), 4.69-4.88 (m, 1H), 5.00-5.20 (m, 3H), 7.26-7.41 (m, 5H); ESI MS m/z 335 (M⁺+1, 20%).

### Step D: Synthesis of benzyl (cis-3-aminocyclopentyl)carbamate hydrochloride

To a solution of benzyl tert-butyl cis-cyclopentane-1,3-diylbiscarbamate (43.5 g) in EtOAc (1.00 L) was added 4 M HCl in EtOAc (350 mL). The mixture was stirred at ambient temperature for 13 h and evaporated under reduced pressure. To the residue was added Et₂O (1.00 L) and the mixture was stirred at ambient temperature for 30 min. The precipitate was collected by filtration, washed with Et₂O (three times) and dried at 80° C. under reduced pressure to give the title compound (35.0 g).

¹H NMR (200 MHz, DMSO-d₆) δ ppm 1.41-2.00 (m, 5H), 2.21-2.39 (m, 1H), 3.38-3.57 (m, 1H), 3.76-3.95 (m, 1H), 5.02 (s, 2H), 7.30-7.41 (m, 5H), 7.52-7.62 (m, 1H); ESI MS m/z 235 [M (free)⁺+1, 100%].

### Step E: Synthesis of benzyl {cis-3-[(4-fluorobenzoyl)amino]cyclopentyl}carbamate

To a solution of benzyl (cis-3-aminocyclopentyl)carbamate (4.50 g) in DMF (50 mL) were added 3-chloro-4-fluorobenzoic acid (2.90 g), Et₃N (2.3 mL), HOBt-H₂O (3.37 g), and EDC-HCl (3.82 g). The mixture was stirred at ambient temperature for 17 h. The mixture was diluted with EtOAc and added saturated aqueous NaHCO₃ (100 mL). The aqueous layer was extracted with EtOAc (three times), and the combined organic layer was washed with water and saturated aqueous NaCl. The organic layer was dried over MgSO₄, filtered, and concentrated under reduced pressure to give the title compound (6.20 g).

¹H NMR (600 MHz, CDCl₃, δ): 1.69-1.91 (m, 3H), 1.91-2.01 (m, 2H), 2.38-2.47 (m, 1H), 3.80-3.91 (m, 1H), 4.40-4.49 (m, 1H), 5.08-5.16 (m, 2H), 5.40 (d, J=6.4 Hz, 1H), 7.10-7.23 (m, 1H), 7.28-7.42 (m, 5H), 7.74-7.85 (m, 1H), 7.95-8.03 (m, 1H), 8.10 (brs, 1H); ESI MS m/z 413 (M⁺+23, 100%).

### Step F: Synthesis of N-(cis-3-aminocyclopentyl)-4-fluorobenzamide

To a solution of benzyl {cis-3-[(4-fluorobenzoyl)amino]cyclopentyl}carbamate (5.78 g) in MeOH (70 mL) and EtOAc (10 ml) was added 10% Pd—C (1.20 g) and the mixture was stirred at 40° C. for 12 h under hydrogen atmosphere. The mixture was filtered, concentrated under reduced pressure to give the crude title compound (3.80 g).

¹H NMR (600 MHz, DMSO-d₆, δ): 1.65-1.72 (m, 1H), 1.78-1.86 (m, 2H), 1.89-1.98 (m, 2H), 2.33-2.41 (m, 1H), 3.48-3.57 (m, 1H), 4.24-4.33 (m, 1H), 7.24-7.33 (m, 2H), 7.97-8.06 (m, 2H), 8.21 (brs, 2H), 8.75 (d, J=6.9 Hz, 1H); ESI MS 77/z 223 (M⁺+1, 100%).

### Step G: Synthesis of 4-fluoro-N-{cis-3-[(6-methylpyridin-2-yl)amino]cyclopentyl}benzamide.

To a suspension of N-(cis-3-aminocyclopentyl)-4-fluorobenzamide (2.00 g) in BuOH (2 mL) was added 2-chloro-6-methylpyridine (1.15 g). The mixture was heated in a microwave synthesizer at 200° C. for 30 min. The mixture was diluted with CHCl₃ and added to saturated aqueous NaHCO₃. The aqueous layer was extracted with CHCl₃ (three times). The combined organic layer was dried over MgSO₄, filtrated, concentrated under reduced pressure, and purified by medium-pressure liquid chromatography (NH-silica gel, 20% to 25% EtOAc in hexane) to give the title compound (471 mg).

¹H NMR (600 MHz, CDCl₃, δ): 1.56-1.66 (m, 1H), 1.71-1.86 (m, 2H), 2.07-2.16 (m, 1H), 2.17-2.27 (m, 1H), 2.37 (s, 3H), 2.50-2.59 (m, 1H), 3.95-4.03 (m, 1H), 4.49-4.58 (m, 1H), 4.67 (d, J=4.1 Hz, 1H), 6.24 (d, J=8.3 Hz, 1H), 6.36 (d, J=7.3 Hz, 1H), 6.50 (d, J=7.3 Hz, 1H), 7.03-7.14 (m, 2H), 7.34-7.42 (m, 1H), 7.69-7.79 (m, 2H); ESI MS m/z 314 (M⁺+1, 100%).

### Example 57

### cis-N-(4-Fluorobenzyl)-N′-(6-methylpyridin-2-yl)cyclopentane-1,3-diamine hydrochloride

To a solution of 4-fluoro-N-{cis-3-[(6-methylpyridin-2-yl)amino]cyclopentyl}benzamide obtained in step G of example 56 (266 mg) in THF (5 mL) was added borane tetrahydrofuran complex (1M THF solution, 7.0 mL), and the mixture was stirred at 80° C. for 1 h. The mixture was concentrated under reduced pressure, and to the residue was added 1M hydrogen chloride in MeOH (12 mL). The mixture was stirred at 80° C. for 1 h and concentrated under reduced pressure. The residue was dissolved in CHCl₃ and 2 M aqueous NaOH, and aqueous layer was extracted with CHCl₃ (twice). The combined organic layer was dried over MgSO₄, filtered, concentrated under reduced pressure. The residue was purified by medium-pressure liquid chromatography (silica gel, 5% to 10% MeOH in CHCl₃) to give a colorless oil (173 mg). To a solution of the above oil in EtOAc (8 mL) was added 4 M hydrogen chloride in EtOAc (1.0 mL). The mixture was stirred at ambient temperature for 30 min, and the precipitate was collected by filtration, washed with EtOAc, and dried at 70° C. under reduced pressure to give the title compound (187 mg).

¹H NMR (600 MHz, DMSO-d₆, δ): 1.76-1.85 (m, 2H), 1.96-2.11 (m, 3H), 2.48 (s, 3H), 2.55-2.63 (m, 1H), 3.50-3.61 (m, 1H), 4.10-4.19 (m, 2H), 4.24-4.41 (m, 1H), 6.66-6.74 (m, 1H), 6.88-7.05 (m, 1H), 7.20-7.36 (m, 2H), 7.62-7.74 (m, 2H), 7.76-7.91 (m, 1H), 9.54-9.84 (m, 2H); ESI MS M/z 300 [M (free)⁺+1, 100%].

### Example 58

### 3,4,5-trifluoro-N-{cis-3-[(6-methylpyridin-2-yl)-amino]cyclopentyl}-benzamide

### Step A: Synthesis of benzyl {cis-3-[(3,4,5-trifluorobenzoyl)amino]cyclopentyl}carbamate

The title compound (4.21 g) was prepared from benzyl (cis-3-aminocyclopentyl)carbamate obtained in step D of example 56 (3.00 g) and 3,4,5-trifluorobenzoic acid (1.95 g) using the procedure for the step E of example 56.

¹H NMR (600 MHz, CDCl₃, δ): 1.68-1.81 (m, 2H), 1.84-1.92 (m, 1H), 1.93-2.04 (m, 2H), 2.37-2.45 (m, 1H), 3.72-3.86 (m, 1H), 4.39-4.50 (m, 1H), 5.05-5.18 (m, 2H), 5.36 (d, J=6.4 Hz, 1H), 7.28-7.42 (m, 5H), 7.55-7.70 (m, 2H), 8.46 (brs, 1H); ESI MS m/z 415 (M⁺+23, 100%).

### Step B: Synthesis of N-(cis-3-aminocyclopentyl)-3,4,5-trifluorobenzamide

The title compound (2.48 g) was prepared from benzyl {cis-3-[(3,4,5-trifluorobenzoyl)-amino]cyclopentyl}carbamate (3.79 g) using the procedure for the step F of example 56.

¹H NMR (600 MHz, DMSO-d₆, δ): 1.28-1.38 (m, 1H), 1.37-1.49 (m, 1H), 1.63-1.71 (m, 1H), 1.72-1.80 (m, 1H), 1.83-1.92 (m, 1H), 2.07-2.15 (m, 1H), 3.21-3.31 (m, 1H), 4.16-4.25 (m, 1H), 7.75-7.84 (m, 2H), 8.65 (brs, 1H); ESI MS m/z 259 (M⁺+1, 100%).

### Step C: Synthesis of 3,4,5-trifluoro-N-{cis-3-[(6-methylpyridin-2-yl)-amino]cyclopentyl}-benzamide

The title compound (58 mg) was prepared from N-(cis-3-aminocyclopentyl)-3,4,5-trifluorobenzamide (1.00 g) and 2-chloro-6-methylpyridine (494 mg) using the procedure for the step G of example 56.

¹H NMR (600 MHz, CDCl₃, δ): 1.67-1.72 (m, 1H), 1.74-1.89 (m, 2H), 2.06-2.16 (m, 1H), 2.18-2.30 (m, 1H), 2.38 (s, 3H), 2.41-2.50 (m, 1H), 3.95-4.05 (m, 1H), 4.49-4.58 (m, 1H), 4.78 (brs, 1H), 6.27 (d, J=8.3 Hz, 1H), 6.45-6.58 (m, 2H), 7.33-7.47 (m, 3H); ESI MS m/z 350 (M⁺+1, 100%).

### Example 59-208

To a suspension of 1-cyclohexyl-3-methylpolystyrene-carbodiimide (150 μL) in CHCl₃ (400 μL) were added N²-(cis-4-aminocyclohexyl)-N⁴,N⁴,6-trimethylpyridine-2,4-diamine obtained in step E of example 28 (30 μmol) in CHCl₃ (200 μL) and carboxylic acid (60 μmol) in CHCl₃ (200 μL) at ambient temperature. After stirring at the same temperature for 13 h, the mixture was filtrated through NH-silica gel. The filtrate was concentrated under reduced pressure, and the residue was purified by silica gel chromatography (silica gel, CHCl₃ to 6% 2 M NH₃/MeOH in CHCl₃) to give the desired product. The product was determined by ESI-MS or APCI-MS.

### Example 209-259

To a solution of half the weight of amide product obtained in example 146-208 in THF (200 μl) was added 1 M borane-THF complex in THF (300 μl). The mixture was stirred at 80° C. for 1 h, and concentrated under reduced pressure. To the residue were added 1 M aqueous HCl (300 μl) and THF (200 μl). The mixture was stirred at 80° C. for 1 h and concentrated under reduced pressure. To the residue was partitioned between CHCl₃ and 2 M aqueous sodium hydroxide. The aqueous layer was extracted with CHCl₃ (300 μL, twice) and EtOAc (300 μL). The combined organic layers were dried over MgSO₄, concentrated under reduced pressure, and purified by silica gel chromatography (silica gel, 33% EtOAc in hexane to 6% 2 M NH₃/MeOH in CHCl₃) to give the desired product. The product was determined by ESI-MS or APCI-MS.

| Ex. No. | compound name | MS |
|---|---|---|
| 59 | 2-(4-acetylphenoxy)-N-(cis-4-{[4-(dimethylamino)-6-methylpyridin-2- | 424 (M + H) |
| | yl]amino}cyclohexyl)acetamide | |
| 60 | N-benzoylglycyl-N-(cis-4-{[4-(dimethylamino)-6-methylpyridin-2- | 467 (M + H) |
| | yl]amino}cyclohexyl)glycinamide | |
| 61 | N-((1S,2R)-2-{[(cis-4-{[4-(dimethylamino)-6-methylpyridin-2- | 478 (M + H) |
| | yl]amino}cyclohexyl)amino]carbonyl}cyclohexyl)benzamide | |
| 62 | 1-[(4-tert-butylphenyl)sulfonyl]-N-(cis-4-{[4-(dimethylamino)-6- | 542 (M + H) |
| | methylpyridin-2-yl]amino}cyclohexyl)-L-prolinamide | |
| 63 | 2-cyclohex-1-en-1-yl-N-(cis-4-{[4-(dimethylamino)-6-methylpyridin-2- | 371 (M + H) |
| | yl]amino}cyclohexyl)acetamide | |
| 64 | 2-cyclohexyl-N-(cis-4-{[4-(dimethylamino)-6-methylpyridin-2- | 373 (M + H) |
| | yl]amino)cyclohexyl)acetamide | |
| 65 | N-(cis-4-{[4-(dimethylamino)-6-methylpyridin-2-yl]amino}cyclohexyl)- | 415 (M + H) |
| | 2-[(4-methylpyrimidin-2-yl)thio]acetamide | |
| 66 | 3-[(4-chlorophenyl)sulfonyl]-N-(cis-4-{[4-(dimethylamino)-6- | 493 (M + H) |
| | methylpyridin-2-yl]amino}cyclohexyl)butanamide | |
| 67 | N-(cis-4-{[4-(dimethylamino)-6-methylpyridin-2-yl]amino}- | 456 (M + H) |
| | cyclohexyl)-5-oxo-1-(2-thienylmethyl)pyrrolidine-3- | |
| | carboxamide | |
| 68 | N-(cis-4-{[4-(dimethylamino)-6-methylpyridin-2-yl]amino}- | 466 (M + H) |
| | cyclohexyl)-2,5-dimethyl-1-(2-thienylmethyl)-1H-pyrrole-3- | |
| | carboxamide | |
| 69 | N-(cis-4-{[4-(dimethylamino)-6-methylpyridin-2-yl]amino}cyclohexyl)- | 435 (M + H) |
| | 2-(4-oxo-3,4-dihydrophthalazin-1-yl)acetamide | |
| 70 | N-(cis-4-{[4-(dimethylamino)-6-methylpyridin-2-yl]amino}cyclohexyl)- | 475 (M + H) |
| | 2-(2-fluorobiphenyl-4-yl)propanamide | |
| 71 | N-{2-[(cis-4-{[4-(dimethylamino)-6-methylpyridin-2- | 400 (M + H) |
| | yl]amino}cyclohexyl)amino]-2-oxoethyl}-2-furamide | |
| 72 | N-(cis-4-{[4-(dimethylamino)-6-methylpyridin-2-yl]amino}cyclohexyl)- | 469 (M + H) |
| | 5-iodo-2-furamide | |
| 73 | N-(cis-4-{[4-(dimethylamino)-6-methylpyridin-2-yl]amino}- | 512 (M + H) |
| | cyclohexyl)-2-[4-(1-oxo-1,3-dihydro-2H-isoindol-2-yl)phenyl]- | |
| | propanamide | |
| 74 | N-(cis-4-{[4-(dimethylamino)-6-methylpyridin-2-yl]amino}cyclohexyl)- | 493 (M + H) |
| | 2-(2-iodophenyl)acetamide | |
| 75 | N-(cis-4-{[4-(dimethylamino)-6-methylpyridin-2-yl]amino}cyclohexyl)- | 392 (M + H) |
| | 1H-indole-3-carboxamide | |
| 76 | N-(cis-4-{[4-(dimethylamino)-6-methylpyridin-2-yl]amino}cyclohexyl)- | 449 (M + H) |
| | 5-(4-methylphenyl)thiophene-3-carboxamide | |
| 77 | N-(cis-4-{[4-(dimethylamino)-6-methylpyridin-2-yl]amino}cyclohexyl)- | 464 (M + H) |
| | 2-(5-methyl-2-phenyl-1,3-thiazol-4-yl)acetamide | |
| 78 | N-(cis-4-{[4-(dimethylamino)-6-methylpyridin-2-yl]amino}- | 451 (M + H) |
| | cyclohexyl)2-(6-methoxy-3-oxo-2,3-dihydro-1H-inden-1-yl)- | |
| | acetamide | |
| 79 | N-(cis-4-{[4-(dimethylamino)-6-methylpyridin-2-yl]amino}cyclohexyl)- | 465 (M + H) |
| | 2-(7-methoxy-2-oxo-2H-chromen-4-yl)acetamide | |
| 80 | N-(cis-4-{[4-(dimethylamino)-6-methylpyridin-2-yl]amino}cyclohexyl)- | 487 (M + H) |
| | 4-[4-(methylsulfonyl)phenyl]-4-oxobutanamide | |
| 81 | N-(cis-4-{[4-(dimethylamino)-6-methylpyridin-2-yl]amino}cyclohexyl)- | 422 (M + H) |
| | 5-methoxy-1H-indole-2-carboxamide | |
| 82 | N-(2,4-difluorophenyl)-2-{2-[(cis-4-{[4-(dimethylamino)-6- | 522 (M + H) |
| | methylpyridin-2-yl]amino}cyclohexyl)amino]-2-oxoethyl}benzamide | |
| 83 | N-(cis-4-{[4-(dimethylamino)-6-methylpyridin-2-yl]amino}cyclohexyl)- | 520 (M - H) |
| | 3-{[(4-methylphenyl)sulfonyl]amino}benzamide | |
| 84 | 2-{2-[(2,5-dimethoxyphenyl)amino]-2-oxoethyl}-N-(cis-4-{[4- | 546 (M + H) |
| | (dimethylamino)-6-methylpyridin-2-yl]amino}cyclohexyl)benzamide | |
| 85 | 2-{2-[(cis-4-{[4-(dimethylamino)-6-methylpyridin-2-yl]amino}- | 528 (M + H) |
| | cyclohexyl)amino]-2-oxoethyl}-N-(4-isopropylphenyl)benzamide | |
| 86 | 2-{2-[(cis-4-{[4-(dimethylamino)-6-methylpyridin-2- | 570 (M + H) |
| | yl]amino}cyclohexyl)amino]-2-oxoethyl}-N-[4- | |
| | (trifluoromethoxy)phenyl]benzamide | |
| 87 | N-(cis-4-{[4-(dimethylamino)-6-methylpyridin-2-yl]amino}cyclohexyl)- | 440 (M + H) |
| | 4-(4-nitrophenyl)butanamide | |
| 88 | N-(cis-4-{[4-(dimethylamino)-6-methylpyridin-2-yl]amino}cyclohexyl)- | 407 (M + H) |
| | 3-oxoindane-1-carboxamide | |
| 89 | N-(cis-4-{[4-(dimethylamino)-6-methylpyridin-2-yl]amino}cyclohexyl)- | 459 (M + H) |
| | 2-(4-phenoxyphenyl)acetamide | |
| 90 | N-(cis-4-{[4-(dimethylamino)-6-methylpyridin-2-yl]amino}cyclohexyl)- | 493 (M + H) |
| | 11-phenylundecanamide | |
| 91 | N-(cis-4-{[4-(dimethylamino)-6-methylpyridin-2-yl]amino}cyclohexyl)- | 400 (M + H) |
| | 2-(pyridin-4-ylthio)acetamide | |
| 92 | N-(cis-4-{[4-(dimethylamino)-6-methylpyridin-2-yl]amino}cyclohexyl)- | 382 (M + H) |
| | N˜2˜-phenylglycinamide | |
| 93 | N-(cis-4-{[4-(dimethylamino)-6-methylpyridin-2-yl]amino}cyclohexyl)- | 475 (M + H) |
| | 2-(4-fluorobenzoyl)benzamide | |
| 94 | N-(cis-4-{[4-(dimethylamino)-6-methylpyridin-2-yl]amino}cyclohexyl)- | 457 (M + H) |
| | 2-(2-phenylethyl)benzamide | |
| 95 | N-(cis-4-{[4-(dimethylamino)-6-methylpyridin-2-yl]amino}cyclohexyl)- | 503 (M + H) |
| | 2-(ethylthio)-2,2-diphenylacetamide | |
| 96 | 2-[(2-cyanophenyl)thio]-N-(cis-4-{[4-(dimethylamino)-6-methylpyridin- | 486 (M + H) |
| | 2-yl]amino}cyclohexyl)benzamide | |
| 97 | N-(cis-4-{[4-(dimethylamino)-6-methylpyridin-2-yl]amino}cyclohexyl)- | 497 (M + H) |
| | 4′-(trifluoromethyl)biphenyl-2-carboxamide | |
| 98 | N-(cis-4-{[4-(dimethylamino)-6-methylpyridin-2-yl]amino}cyclohexyl)- | 486 (M + H) |
| | 7-nitro-9H-fluorene-4-carboxamide | |
| 99 | (2S)-2-(3-benzoylphenyl)-N-(cis-4-{[4-(dimethylamino)-6- | 485 (M + H) |
| | methylpyridin-2-yl]amino}cyclohexyl)propanamide | |
| 100 | N′-(cis-4-{[4-(dimethylamino)-6-methylpyridin-2- | 604 (M + H) |
| | yl]amino}cyclohexyl)-N,N-bis[(1S)-1-phenylethyl]phthalamide | |
| 101 | 2-[(4-chlorophenyl)thio]-N-(cis-4-{[4-(dimethylamino)-6- | 565 (M + H) |
| | methylpyridin-2-yl]amino}cyclohexyl)-4-(4-methylphenyl)-4- | |
| | oxobutanamide | |
| 102 | N-(cis-4-{[4-(dimethylamino)-6-methylpyridin-2-yl]amino}- | 549 (M + H) |
| | cyclohexyl)-4-(4-fluorophenyl)-2-[(4-methylphenyl)thio]-4- | |
| | oxobutanamide | |
| 103 | N-(cis-4-{[4-(dimethylamino)-6-methylpyridin-2-yl]amino}cyclohexyl)- | 491 (M + H) |
| | 2-[4-(2-thienylcarbonyl)phenyl]propanamide | |
| 104 | N-(cis-4-{[4-(dimethylamino)-6-methylpyridin-2-yl]amino}cyclohexyl)- | 451 (M + H) |
| | 2-[4-(trifluoromethoxy)phenyl]acetamide | |
| 105 | N-(cis-4-{[4-(dimethylamino)-6-methylpyridin-2-yl]amino}cyclohexyl)- | 387 (M + H) |
| | 4,4,4-trifluoro-3-methylbutanamide | |
| 106 | N-(cis-4-{[4-(dimethylamino)-6-methylpyridin-2-yl]amino}cyclohexyl)- | 415 (M + H) |
| | 5-(2-thienyl)pentanamide | |
| 107 | N-(cis-4-{[4-(dimethylamino)-6-methylpyridin-2-yl]amino}cyclohexyl)- | 460 (M + H) |
| | N˜2˜-[(4-methylphenyl)sulfonyl]glycinamide | |
| 108 | 2-[5-(benzyloxy)-1H-indol-3-yl]-N-(cis-4-{[4-(dimethylamino)-6- | 512 (M + H) |
| | methylpyridin-2-yl]amino}cyclohexyl)acetamide | |
| 109 | N-(cis-4-{[4-(dimethylamino)-6-methylpyridin-2-yl]amino}cyclohexyl)- | 486 (M + H) |
| | N′-(3-methylphenyl)phthalamide | |
| 110 | N-(cis-4-{[4-(dimethylamino)-6-methylpyridin-2-yl]amino}- | 511 (M + H) |
| | cyclohexyl)-3-methyl-4-oxo-2-phenyl-4H-chromene-8- | |
| | carboxamide | |
| 111 | benzyl 3-[(cis-4-{[4-(dimethylamino)-6-methylpyridin-2- | 501 (M + H) |
| | yl]amino}cyclohexyl)amino}-3-oxo-2-phenylpropanoate | |
| 112 | 2-[3,5-bis(trifluoromethyl)benzoyl]-N-(cis-4-{[4-(dimethylamino)-6- | 593 (M + H) |
| | methylpyridin-2-yl]amino}cyclohexyl)benzamide | |
| 113 | N-(cis-4-{[4-(dimethylamino)-6-methylpyridin-2-yl]amino}- | 527 (M + H) |
| | cyclohexyl)-2-[(3-methyl-1-benzothien-2-yl)carbonyl]- | |
| | benzamide | |
| 114 | N-(cis-4-{[4-(dimethylamino)-6-methylpyridin-2-yl]amino}cyclohexyl)- | 481 (M + H) |
| | 3-phenylcinnoline-4-carboxamide | |
| 115 | N-(cis-4-{[4-(dimethylamino)-6-methylpyridin-2-yl]amino}cyclohexyl)- | 455 (M + H) |
| | 9-oxo-9H-fluorene-2-carboxamide | |
| 116 | N-(cis-4-{[4-(dimethylamino)-6-methylpyridin-2- | 429 (M + H) |
| | yl]amino}cyclohexyl)biphenyl-2-carboxamide | |
| 117 | N-(cis-4-{[4-(dimethylamino)-6-methylpyridin-2-yl]amino}cyclohexyl)- | 445 (M + H) |
| | 4-phenoxybenzamide | |
| 118 | N-(cis-4-{[4-(dimethylamino)-6-methylpyridin-2-yl]amino}cyclohexyl)- | 457 (M + H) |
| | 9H-xanthene-9-carboxamide | |
| 119 | N-(cis-4-{[4-(dimethylamino)-6-methylpyridin-2-yl]amino}cyclohexyl)- | 500 (M + H) |
| | N′-[(1S)-1-phenylethyl]phthalamide | |
| 120 | 4-(benzyloxy)-N-(cis-4-{[4-(dimethylamino)-6-methylpyridin-2- | 459 (M + H) |
| | yl]amino}cyclohexyl)benzamide | |
| 121 | N-(cis-4-{[4-(dimethylamino)-6-methylpyridin-2-yl]amino}cyclohexyl)- | 471 (M + H) |
| | 2-(4-methylbenzoyl)benzamide | |
| 122 | N-(cis-4-{[4-(dimethylamino)-6-methylpyridin-2-yl]amino}cyclohexyl)- | 459 (M + H) |
| | 2-(phenoxymethyl)benzamide | |
| 123 | N-(cis-4-{[4-(dimethylamino)-6-methylpyridin-2-yl]amino}cyclohexyl)- | 522 (M + H) |
| | N′-1-naphthylphthalamide | |
| 124 | N-(cis-4-{[4-(dimethylamino)-6-methylpyridin-2- | 453 (M + H) |
| | yl]amino}cyclohexyl)anthracene-2-carboxamide | |
| 125 | N-(cis-4-{[4-(dimethylamino)-6-methylpyridin-2-yl]amino}cyclohexyl)- | 527 (M + H) |
| | 4′-heptylbiphenyl-4-carboxamide | |
| 126 | 2-[4-(4-chlorophenyl)-2-phenyl-1,3-thiazol-5-yl]-N-(cis-4-{[4- | 560 (M + H) |
| | (dimethylamino)-6-methylpyridin-2-yl]amino}cyclohexyl)acetamide | |
| 127 | 2-(benzylthio)-N-(cis-4-{[4-(dimethylamino)-6-methylpyridin-2- | 413 (M + H) |
| | yl]amino}cyclohexyl)acetamide | |
| 128 | N-(cis-4-{[4-(dimethylamino)-6-methylpyridin-2-yl]amino}cyclohexyl)- | 395 (M + H) |
| | 4-phenylbutanamide | |
| 129 | 2-(1-benzothien-3-yl)-N-(cis-4-{[4-(dimethylamino)-6-methylpyridin-2- | 423 (M + H) |
| | yl]amino}cyclohexyl)acetamide | |
| 130 | 2-(2,3-dihydro-1H-inden-2-yl)-N-(cis-4-{[4-(dimethylamino)-6- | 407 (M + H) |
| | methylpyridin-2-yl]amino}cyclohexyl)acetamide | |
| 131 | 4-(3,4-dimethoxyphenyl)-N-(cis-4-{[4-(dimethylamino)-6- | 455 (M + H) |
| | methylpyridin-2-yl]amino}cyclohexyl)butanamide | |
| 132 | 4-(2,3-dihydro-1,4-benzodioxin-6-yl)-N-(cis-4-{[4-(dimethylamino)-6- | 453 (M + H) |
| | methylpyridin-2-yl]amino}cyclohexyl)butanamide | |
| 133 | N-(cis-4-{[4-(dimethylamino)-6-methylpyridin-2-yl]amino}- | 496 (M + H) |
| | cyclohexyl)-1-[(4-methylphenyl)sulfonyl]-1H-pyrrole-3- | |
| | carboxamide | |
| 134 | N-(cis-4-{[4-(dimethylamino)-6-methylpyridin-2-yl]amino}cyclohexyl)- | 431 (M + H) |
| | 4-(methylsulfonyl)benzamide | |
| 135 | 5-acetyl-N-(cis-4-{[4-(dimethylamino)-6-methylpyridin-2- | 401 (M + H) |
| | yl]amino}cyclohexyl)thiophene-2-carboxamide | |
| 136 | 3-chloro-N-(cis-4-{[4-(dimethylamino)-6-methylpyridin-2- | 545 (M + H) |
| | yl]amino}cyclohexyl)-4-(isopropylsulfonyl)-5-(methylthio)thiophene-2- | |
| | carboxamide | |
| 137 | N-(cis-4-{[4-(dimethylamino)-6-methylpyridin-2-yl]amino}cyclohexyl)- | 437 (M + H) |
| | 5-(methylsulfonyl)thiophene-2-carboxamide | |
| 138 | N-(cis-4-{[4-(dimethylamino)-6-methylpyridin-2-yl]amino}cyclohexyl)- | 420 (M + H) |
| | 4-(1,3-oxazol-5-yl)benzamide | |
| 139 | N-(cis-4-{[4-(dimethylamino)-6-methylpyridin-2-yl]amino}cyclohexyl)- | 532 (M + H) |
| | 1-(phenylsulfonyl)-1H-indole-3-carboxamide | |
| 140 | N-(cis-4-{[4-(dimethylamino)-6-methylpyridin-2-yl]amino}cyclohexyl)- | 399 (M + H) |
| | 4-nitropyridine-2-carboxamide | |
| 141 | N-(cis-4-{[4-(dimethylamino)-6-methylpyridin-2-yl]amino}cyclohexyl)- | 381 (M + H) |
| | 2-oxo-2-phenylacetamide | |
| 142 | N-(cis-4-{[4-(dimethylamino)-6-methylpyridin-2-yl]amino}cyclohexyl)- | 423 (M + H) |
| | 2-mesityl-2-oxoacetamide | |
| 143 | (3E)-N-(cis-4-{[4-(dimethylamino)-6-methylpyridin-2- | 407 (M + H) |
| | yl]amino}cyclohexyl)-2-oxo-4-phenylbut-3-enamide | |
| 144 | (1S,2R,5S)-2-benzoyl-N-(cis-4-{[4-(dimethylamino)-6-methylpyridin-2- | 539 (M + H) |
| | yl]amino}cyclohexyl)-5-phenylcyclohexanecarboxamide | |
| 145 | N-(cis-4-{[4-(dimethylamino)-6-methylpyridin-2-yl]amino}cyclohexyl)- | 453 (M + H) |
| | 2-(9H-fluoren-9-ylidene)acetamide | |
| 146 | N-(cis-4-{[4-(dimethylamino)-6-methylpyridin-2-yl]amino}cyclohexyl)- | 395 (M + H) |
| | 2,1,3-benzoxadiazole-5-carboxamide | |
| 147 | 2-(4-chlorophenoxy)-N-(cis-4-{[4-(dimethylamino)-6-methylpyridin-2- | 417 (M + H) |
| | yl]amino}cyclohexyl)acetamide | |
| 148 | methyl 4-{[(cis-4-{[4-(dimethylamino)-6-methylpyridin-2- | 411 (M + H) |
| | yl]amino}cyclohexyl)amino]carbonyl}benzoate | |
| 149 | N-(cis-4-{[4-(dimethylamino)-6-methylpyridin-2-yl]amino}cyclohexyl)- | 383 (M + H) |
| | 2-phenoxyacetamide | |
| 150 | N-(cis-4-{[4-(dimethylamino)-6-methylpyridin-2-yl]amino}cyclohexyl)- | 397 (M + H) |
| | 1,3-benzodioxole-5-carboxamide | |
| 151 | N-(cis-4-{[4-(dimethylamino)-6-methylpyridin-2-yl]amino}- | 487 (M + H) |
| | cyclohexyl)-1-phenyl-5-(trifluoromethyl)-1H-pyrazole-4- | |
| | carboxamide | |
| 152 | N-(cis-4-{[4-(dimethylamino)-6-methylpyridin-2-yl]amino}cyclohexyl)- | 428 (M + H) |
| | 2-(2-nitrophenoxy)acetamide | |
| 153 | N-(cis-4-{[4-(dimethylamino)-6-methylpyridin-2- | 405 (M + H) |
| | yl]amino}cyclohexyl)quinoxaline-2-carboxamide | |
| 154 | N-(cis-4-{[4-(dimethylamino)-6-methylpyridin-2-yl]amino}cyclohexyl)- | 367 (M + H) |
| | 4-methylbenzamide | |
| 155 | N-(cis-4-{[4-(dimethylamino)-6-methylpyridin-2-yl]amino}cyclohexyl)- | 473 (M + H) |
| | 2-(pentafluorophenoxy)acetamide | |
| 156 | 2-(3,4-dimethoxyphenyl)-N-(cis-4-{[4-(dimethylamino)-6- | 427 (M + H) |
| | methylpyridin-2-yl]amino}cyclohexyl)acetamide | |
| 157 | N-(cis-4-{[4-(dimethylamino)-6-methylpyridin-2-yl]amino}cyclohexyl)- | 467 (M + H) |
| | 4-(heptyloxy)benzamide | |
| 158 | N-(cis-4-{[4-(dimethylamino)-6-methylpyridin-2-yl]amino}cyclohexyl)- | 423 (M + H) |
| | 4-pentylbenzamide | |
| 159 | N-(cis-4-{[4-(dimethylamino)-6-methylpyridin-2-yl]amino}cyclohexyl)- | 479 (M + H) |
| | 4-nonylbenzamide | |
| 160 | N-(cis-4-{[4-(dimethylamino)-6-methylpyridin-2-yl]amino}cyclohexyl)- | 347 (M + H) |
| | 3,3-dimethylbutanamide | |
| 161 | N-(cis-4-{[4-(dimethylamino)-6-methylpyridin-2-yl]amino}- | 586 (M + H) |
| | cyclohexyl)-Nalpha-(1-naphthylsulfonyl)-L- | |
| | phenylalaninamide | |
| 162 | N-(cis-4-{[4-(dimethylamino)-6-methylpyridin-2-yl]amino}- | 581 (M + H) |
| | cyclohexyl)-Nalpha-[(4-nitrophenyl)sulfonyl]-L- | |
| | phenylalaninamide | |
| 163 | N-(cis-4-{[4-(dimethylamino)-6-methylpyridin-2-yl]amino}cyclohexyl)- | 453 (M + H) |
| | 4-[(trifluoromethyl)thio]benzamide | |
| 164 | (2E)-N-(cis-4-{[4-(dimethylamino)-6-methylpyridin-2- | 447 (M + H) |
| | yl]amino}cyclohexyl)-3-[3-(trifluoromethyl)phenyl]acrylamide | |
| 165 | (2E)-3-(2-chlorophenyl)-N-(cis-4-{[4-(dimethylamino)-6- | 413 (M + H) |
| | methylpyridin-2-yl]amino}cyclohexyl)acrylamide | |
| 166 | N-(cis-4-{[4-(dimethylamino)-6-methylpyridin-2-yl]amino}cyclohexyl)- | 516 (M + H) |
| | 4-[(dipropylamino)sulfonyl]benzamide | |
| 167 | 2-(4-chlorophenoxy)-N-(cis-4-{[4-(dimethylamino)-6-methylpyridin-2- | 445 (M + H) |
| | yl]amino}cyclohexyl)-2-methylpropanamide | |
| 168 | N-(cis-4-{[4-(dimethylamino)-6-methylpyridin-2-yl]amino}cyclohexyl)- | 417 (M + H) |
| | 2-(1-naphthyl)acetamide | |
| 169 | (2E)-N-(cis-4-{[4-(dimethylamino)-6-methylpyridin-2- | 379 (M + H) |
| | yl]amino}cyclohexyl)-3-phenylacrylamide | |
| 170 | N-(cis-4-{[4-(dimethylamino)-6-methylpyridin-2- | 317 (M + H) |
| | yl]amino}cyclohexyl)cyclopropanecarboxamide | |
| 171 | N-(cis-4-{[4-(dimethylamino)-6-methylpyridin-2-yl]amino}cyclohexyl)- | 389 (M + H) |
| | 3,4-difluorobenzamide | |
| 172 | N-(cis-4-{[4-(dimethylamino)-6-methylpyridin-2-yl]amino}cyclohexyl)- | 413 (M + H) |
| | 3,4-dimethoxybenzamide | |
| 173 | N-(cis-4-{[4-(dimethylamino)-6-methylpyridin-2-yl]amino}cyclohexyl)- | 397 (M + H) |
| | 4-ethoxybenzamide | |
| 174 | N-(cis-4-{[4-(dimethylamino)-6-methylpyridin-2-yl]amino}cyclohexyl)- | 479 (M + H) |
| | 2-iodobenzamide | |
| 175 | N-(cis-4-{[4-(dimethylamino)-6-methylpyridin-2-yl]amino}cyclohexyl)- | 400 (M + H) |
| | 2-(methylthio)nicotinamide | |
| 176 | N-(cis-4-{[4-(dimethylamino)-6-methylpyridin-2-yl]amino}cyclohexyl)- | 403 (M + H) |
| | 2-naphthamide | |
| 177 | N-(cis-4-{[4-(dimethylamino)-6-methylpyridin-2-yl]amino}cyclohexyl)- | 421 (M + H) |
| | 3-(trifluoromethyl)benzamide | |
| 178 | N-(cis-4-{[4-(dimethylamino)-6-methylpyridin-2-yl]amino}cyclohexyl)- | 399 (M + H) |
| | 2-(phenylthio)acetamide | |
| 179 | N-(cis-4-{[4-(dimethylamino)-6-methylpyridin-2-yl]amino}cyclohexyl)- | 437 (M + H) |
| | 4-(trifluoromethoxy)benzamide | |
| 180 | N-(cis-4-{[4-(dimethylamino)-6-methylpyridin-2-yl]amino}cyclohexyl)- | 439 (M + H) |
| | 4-fluoro-2-(trifluoromethyl)benzamide | |
| 181 | 3-chloro-N-(cis-4-{[4-(dimethylamino)-6-methylpyridin-2- | 405 (M + H) |
| | yl]amino}cyclohexyl)-4-fluorobenzamide | |
| 182 | 2-(2-bromophenyl)-N-(cis-4-{[4-(dimethylamino)-6-methylpyridin-2- | 445 (M + H) |
| | yl]amino}cyclohexyl)acetamide | |
| 183 | N-(cis-4-{[4-(dimethylamino)-6-methylpyridin-2- | 359 (M + H) |
| | yl]amino}cyclohexyl)cyclohexanecarboxamide | |
| 184 | 2-cyclopentyl-N-(cis-4-{[4-(dimethylamino)-6-methylpyridin-2- | 359 (M + H) |
| | yl]amino}cyclohexyl)acetamide | |
| 185 | 4-chloro-N-(cis-4-{[4-(dimethylamino)-6-methylpyridin-2- | 387 (M + H) |
| | yl]amino}cyclohexyl)benzamide | |
| 186 | 1-(4-chlorophenyl)-N-(cis-4-{[4-(dimethylamino)-6-methylpyridin-2- | 455 (M + H) |
| | yl]amino}cyclohexyl)cyclopentanecarboxamide | |
| 187 | 4-cyano-N-(cis-4-{[4-(dimethylamino)-6-methylpyridin-2- | 378 (M + H) |
| | yl]amino}cyclohexyl)benzamide | |
| 188 | 6-chloro-N-(cis-4-{[4-(dimethylamino)-6-methylpyridin-2- | 441 (M + H) |
| | yl]amino}cyclohexyl)-2H-chromene-3-carboxamide | |
| 189 | (2E)-N-(cis-4-{[4-(dimethylamino)-6-methylpyridin-2- | 424 (M + H) |
| | yl]amino}cyclohexyl)-3-(4-nitrophenyl)acrylamide | |
| 190 | N-(cis-4-{[4-(dimethylamino)-6-methylpyridin-2-yl]amino}cyclohexyl)- | 398 (M + H) |
| | 3-nitrobenzamide | |
| 191 | N-(cis-4-{[4-(dimethylamino)-6-methylpyridin-2-yl]amino}cyclohexyl)- | 455 (M + H) |
| | 9-oxo-9H-fluorene-4-carboxamide | |
| 192 | N-(cis-4-{[4-(dimethylamino)-6-methylpyridin-2-yl]amino}cyclohexyl)- | 520 (M + H) |
| | 2-(4-methoxyphenoxy)-5-nitrobenzamide | |
| 193 | N-(cis-4-{[4-(dimethylamino)-6-methylpyridin-2-yl]amino}- | 483 (M + H) |
| | cyclohexyl)-9,10-dioxo-9,10-dihydroanthracene-2- | |
| | carboxamide | |
| 194 | N-(cis-4-{[4-(dimethylamino)-6-methylpyridin-2- | 429 (M + H) |
| | yl]amino}cyclohexyl)biphenyl-4-carboxamide | |
| 195 | 2-[4-(benzyloxy)phenyl]-N-(cis-4-{[4-(dimethylamino)-6- | 473 (M + H) |
| | methylpyridin-2-yl]amino}cyclohexyl)acetamide | |
| 196 | 2-(benzyloxy)-N-(cis-4-{[4-(dimethylamino)-6-methylpyridin-2- | 397 (M + H) |
| | yl]amino}cyclohexyl)acetamide | |
| 197 | 4-[(4-chlorophenyl)sulfonyl]-N-(cis-4-{[4-(dimethylamino)-6- | 547 (M + H) |
| | methylpyridin-2-yl]amino}cyclohexyl)-3-methylthiophene-2- | |
| | carboxamide | |
| 198 | N-(cis-4-{[4-(dimethylamino)-6-methylpyridin-2- | 345 (M + H) |
| | yl]amino}cyclohexyl)cyclopentanecarboxamide | |
| 199 | 4-bromo-N-(cis-4-{[4-(dimethylamino)-6-methylpyridin-2- | 431 (M + H) |
| | yl]amino}cyclohexyl)benzamide | |
| 200 | 4-(dimethylamino)-N-(cis-4-{[4-(dimethylamino)-6-methylpyridin-2- | 396 (M + H) |
| | yl]amino}cyclohexyl)benzamide | |
| 201 | 2-(4-chlorophenyl)-N-(cis-4-{[4-(dimethylamino)-6-methylpyridin-2- | 401 (M + H) |
| | yl]amino}cyclohexyl)acetamide | |
| 202 | N-(cis-4-{[4-(dimethylamino)-6-methylpyridin-2-yl]amino}cyclohexyl)- | 381 (M + H) |
| | 3-phenylpropanamide | |
| 203 | N-(cis-4-{[4-(dimethylamino)-6-methylpyridin-2-yl]amino}cyclohexyl)- | 446 (M + H) |
| | 2-phenoxynicotinamide | |
| 204 | 5-bromo-N-(cis-4-{[4-(dimethylamino)-6-methylpyridin-2- | 432 (M + H) |
| | yl]amino}cyclohexyl)nicotinamide | |
| 205 | N-(cis-4-{[4-(dimethylamino)-6-methylpyridin-2- | 354 (M + H) |
| | yl]amino}cyclohexyl)isonicotinamide | |
| 206 | N-(cis-4-{[4-(dimethylamino)-6-methylpyridin-2-yl]amino}cyclohexyl)- | 409 (M + H) |
| | 1-benzothiophene-2-carboxamide | |
| 207 | N-(cis-4-{[4-(dimethylamino)-6-methylpyridin-2-yl]amino}cyclohexyl)- | 343 (M + H) |
| | 2-furamide | |
| 208 | N-(cis-4-{[4-(dimethylamino)-6-methylpyridin-2- | 359 (M + H) |
| | yl]amino}cyclohexyl)thiophene-2-carboxamide | |
| 209 | N-(cis-4-{[2-(4-chlorophenoxy)ethyl]amino}cyclohexyl)-N,N-6- | 403 (M + H) |
| | trimethylpyridine-2,4-diamine | |
| 210 | methyl 4-{[(cis-4-{[4-(dimethylamino)-6-methylpyridin-2- | 397 (M + H) |
| | yl]amino}cyclohexyl)amino]methyl}benzoate | |
| 211 | N-{cis-4-[(1,3-benzodioxol-5-ylmethyl)amino]cyclohexyl}-N,N-6- | 383 (M + H) |
| | trimethylpyridine-2,4-diamine | |
| 212 | N,N-6-trimethyl-N-[cis-4-({[1-phenyl-5-(trifluoromethyl)-1H-pyrazol-4- | 473 (M + H) |
| | yl]methyl}amino)cyclohexyl]pyridine-2,4-diamine | |
| 213 | N,N-6-trimethyl-N-(cis-4-{[2-(2- | 414 (M + H) |
| | nitrophenoxy)ethyl]amino}cyclohexyl)pyridine-2,4-diamine | |
| 214 | N,N-6-trimethyl-N-{cis-4-[(4- | 353 (M + H) |
| | methylbenzyl)amino]cyclohexyl}pyridine-2,4-diamine | |
| 215 | N,N-6-trimethyl-N-(cis-4-{[2- | 459 (M + H) |
| | (pentafluorophenoxy)ethyl]amino}cyclohexyl)pyridine-2,4-diamine | |
| 216 | N-(cis-4-{[2-(3,4-dimethoxyphenyl)ethyl]amino}cyclohexyl)-N,N-6- | 413 (M + H) |
| | trimethylpyridine-2,4-diamine | |
| 217 | N-(cis-4-{[4-(heptyloxy)benzyl]amino}cyclohexyl)-N,N-6- | 453 (M + H) |
| | trimethylpyridine-2,4-diamine | |
| 218 | N,N-6-trimethyl-N-{cis-4-[(4-pentylbenzyl)amino]cyclohexyl}pyridine- | 409 (M + H) |
| | 2,4-diamine | |
| 219 | N,N-6-trimethyl-N-{cis-4-[(4-nonylbenzyl)amino]cyclohexyl}pyridine- | 465 (M + H) |
| | 2,4-diamine | |
| 220 | N-{cis-4-[(3,3-dimethylbutyl)amino]cyclohexyl}-N,N-6- | 333 (M + H) |
| | trimethylpyridine-2,4-diamine | |
| 221 | N-{(1S)-1-benzyl-2-[(cis-4-{[4-(dimethylamino)-6-methylpyridin-2- | 572 (M + H) |
| | yl]amino}cyclohexyl)amino]ethyl}naphthalene-1-sulfonamide | |
| 222 | N-{(1S)-1-benzyl-2-[(cis-4-{[4-(dimethylamino)-6-methylpyridin-2- | 567 (M + H) |
| | yl]amino}cyclohexyl)amino]ethyl}-4-nitrobenzenesulfonamide | |
| 223 | N,N-6-trimethyl-N-[cis-4-({4-[(trifluoromethyl)thio]benzyl}- | 439 (M + H) |
| | amino)cyclohexyl]pyridine-2,4-diamine | |
| 224 | N-(cis-4-{[(2E)-3-(2-chlorophenyl)prop-2-en-1-yl]amino}cyclohexyl)- | 399 (M + H) |
| | N,N-6-trimethylpyridine-2,4-diamine | |
| 225 | 4-{[(cis-4-{[4-(dimethylamino)-6-methylpyridin-2-yl]amino}- | 502 (M + H) |
| | cyclohexyl)amino]methyl}-N,N-dipropylbenzenesulfonamide | |
| 226 | N-(cis-4-{[2-(4-chlorophenoxy)-2-methylpropyl]amino}cyclohexyl)- | 431 (M + H) |
| | N,N-6-trimethylpyridine-2,4-diamine | |
| 227 | N,N-6-trimethyl-N-(cis-4-{[2-(1- | 403 (M + H) |
| | naphthyl)ethyl]amino}cyclohexyl)pyridine-2,4-diamine | |
| 228 | N,N-6-trimethyl-N-(cis-4-{[(2E)-3-phenylprop-2-en-1- | 365 (M + H) |
| | yl]amino}cyclohexyl)pyridine-2,4-diamine | |
| 229 | N-{cis-4-[(3,4-difluorobenzyl)amino]cyclohexyl}-N,N-6- | 375 (M + H) |
| | trimethylpyridine-2,4-diamine | |
| 230 | N-{cis-4-[(3,4-dimethoxybenzyl)amino]cyclohexyl}-N,N-6- | 399 (M + H) |
| | trimethylpyridine-2,4-diamine | |
| 231 | N-{cis-4-[(4-ethoxybenzyl)amino]cyclohexyl}-N,N-6- | 383 (M + H) |
| | trimethylpyridine-2,4-diamine | |
| 232 | N-{cis-4-[(2-iodobenzyl)amino]cyclohexyl}-N,N-6-trimethylpyridine- | 465 (M + H) |
| | 2,4-diamine | |
| 233 | N,N-6-trimethyl-N-{cis-4-[(2- | 389 (M + H) |
| | naphthylmethyl)amino]cyclohexyl}pyridine-2,4-diamine | |
| 234 | N,N-6-trimethyl-N-(cis-4-{[3- | 407 (M + H) |
| | (trifluoromethyl)benzyl]amino}cyclohexyl)pyridine-2,4-diamine | |
| 235 | N,N-6-trimethyl-N-(cis-4-{[2- | 385 (M + H) |
| | (phenylthio)ethyl]amino}cyclohexyl)pyridine-2,4-diamine | |
| 236 | N,N-6-trimethyl-N-(cis-4-{[4- | 423 (M + H) |
| | (trifluoromethoxy)benzyl]amino}cyclohexyl)pyridine-2,4-diamine | |
| 237 | N-(cis-4-{[4-fluoro-2-(trifluoromethyl)benzyl]amino}cyclohexyl)-N,N- | 425 (M + H) |
| | 6-trimethylpyridine-2,4-diamine | |
| 238 | N-{cis-4-[(3-chloro-4-fluorobenzyl)amino]cyclohexyl}-N,N-6- | 391 (M + H) |
| | trimethylpyridine-2,4-diamine | |
| 239 | N-(cis-4-{[2-(2-bromophenyl)ethyl]amino}cyclohexyl)-N,N-6- | 431 (M + H) |
| | trimethylpyridine-2,4-diamine | |
| 240 | N-{cis-4-[(cyclohexylmethyl)amino]cyclohexyl}-N,N-6- | 345 (M + H) |
| | trimethylpyridine-2,4-diamine | |
| 241 | N-{cis-4-[(2-cyclopentylethyl)amino]cyclohexyl}-N,N-6- | 345 (M + H) |
| | trimethylpyridine-2,4-diamine | |
| 242 | N-{cis-4-[(4-chlorobenzyl)amino]cyclohexyl}-N,N-6-trimethylpyridine- | 373 (M + H) |
| | 2,4-diamine | |
| 243 | N-[cis-4-({[1-(4-chlorophenyl)cyclopentyl]methyl}amino)cyclohexyl]- | 441(M + H) |
| | N,N-6-trimethylpyridine-2,4-diamine | |
| 244 | N-(cis-4-{[(6-chloro-2H-chromen-3-yl)methyl]amino}cyclohexyl)-N,N- | 427 (M + H) |
| | 6-trimethylpyridine-2,4-diamine | |
| 245 | N,N-6-trimethyl-N-(cis-4-{[(2E)-3-(4-nitrophenyl)prop-2-en-1- | 410 (M + H) |
| | yl]amino}cyclohexyl)pyridine-2,4-diamine | |
| 246 | N,N-6-trimethyl-N-{cis-4-[(3-nitrobenzyl)amino]cyclohexyl}pyridine- | 384 (M + H) |
| | 2,4-diamine | |
| 247 | N-(cis-4-{[2-(4-methoxyphenoxy)-5-nitrobenzyl]amino}cyclohexyl)- | 506 (M + H) |
| | N,N-6-trimethylpyridine-2,4-diamine | |
| 248 | N-{cis-4-[(biphenyl-4-ylmethyl)amino]cyclohexyl}-N,N-6- | 415 (M + H) |
| | trimethylpyridine-2,4-diamine | |
| 249 | N-[cis-4-({2-[4-(benzyloxy)phenyl]ethyl}amino)cyclohexyl]-N,N-6- | 459 (M + H) |
| | trimethylpyridine-2,4-diamine | |
| 250 | N-(cis-4-{[2-(benzyloxy)ethyl]amino}cyclohexyl)-N,N-6- | 383 (M + H) |
| | trimethylpyridine-2,4-diamine | |
| 251 | N-{cis-4-[({4-[(4-chlorophenyl)sulfonyl]-3-methyl-2- | 533 (M + H) |
| | thienyl}methyl)amino]cyclohexyl}-N,N-6-trimethylpyridine- | |
| | 2,4-diamine | |
| 252 | N-{cis-4-[(4-bromobenzyl)amino]cyclohexyl}-N,N-6-trimethylpyridine- | 417 (M + H) |
| | 2,4-diamine | |
| 253 | N-(cis-4-{[2-(4-chlorophenyl)ethyl]amino}cyclohexyl)-N,N-6- | 387 (M + H) |
| | trimethylpyridine-2,4-diamine | |
| 254 | N,N-6-trimethyl-N-{cis-4-[(3-phenylpropyl)amino]cyclohexyl}pyridine- | 367 (M + H) |
| | 2,4-diamine | |
| 255 | N,N-6-trimethyl-N-(cis-4-{[(2-phenoxypyridin-3 - | 432 (M + H) |
| | yl)methyl]amino}cyclohexyl)pyridine-2,4-diamine | |
| 256 | N,N-6-trimethyl-N-{cis-4-[(pyridin-4- | 340 (M + H) |
| | ylmethyl)amino]cyclohexyl}pyridine-2,4-diamine | |
| 257 | N-{cis-4-[(1-benzothien-2-ylmethyl)amino]cyclohexyl}-N,N-6- | 395 (M + H) |
| | trimethylpyridine-2,4-diamine | |
| 258 | N-{cis-4-[(2-furylmethyl)amino]cyclohexyl}-N,N-6-trimethylpyridine- | 329 (M + H) |
| | 2,4-diamine | |
| 259 | N,N-6-trimethyl-N-{cis-4-[(2- | 345 (M + H) |
| | thienylmethyl)amino]cyclohexyl}pyridine-2,4-diamine | |

Assay Procedures

Assay for Determination of Constitutive Activity of Non-Endogenous GPCRs

### Example 260

### Intracellular IP₃ Accumulation Assay

On day 1, cells to be transfected can be plated onto 24 well plates, usually 1×10⁵ cells/well (although this number can be optimized. On day 2 cells can be transfected by firstly mixing 0.25 μg DNA (e.g., pCMV vector or pCMV vector comprising polynucleotide encoding receptor) in 50 μl serum free DMEM/well and 2 μl lipofectamine in 50 μl serum-free DMEM/well. The solutions are gently mixed and incubated for 15-30 min at room temperature. Cells are washed with 0.5 mL PBS and 400 μl of serum free media is mixed with the transfection media and added to the cells. The cells are then incubated for 3-4 hrs at 37° C./5% CO₂ and then the transfection media is removed and replaced with 1 mL/well of regular growth media. On day 3 the cells are labeled with ³H-myo-inositol. Briefly, the media is removed and the cells are washed with 0.5 mL PBS. Then 0.5 mL inositol-free/serum free media (GIBCO BRL) is added/well with 0.25 μCi of ³H-myo-inositol/well and the cells are incubated for 16-18 hrs o/n at 37° C./5% CO₂ On Day 4 the cells are washed with 0.5 mL PBS and 0.45 mL of assay medium is added containing inositol-free/serum free media 10 μM pargyline 10 mM lithium chloride or 0.4 mL of assay medium and 50 μl of 10× ketanserin (ket) to final concentration of 10 μM. The cells are then incubated for 30 min at 37° C. The cells are then washed with 0.5 mL PBS and 200 μl of fresh/ice cold stop solution (1 M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCl). The lysate is then transferred into 1.5 mL eppendorf tubes and 1 mL of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 mL of upper phase is loaded onto the column. The column is washed with 10 mL of 5 mM myo-inositol and 10 mL of 5 mM Na-borate/60 mM Na-formate. The inositol tris phosphates are eluted into scintillation vials containing 10 mL of scintillation cocktail with 2 mL of 0.1 M formic acid/1 M ammonium formate. The columns are regenerated by washing with 10 mL of 0.1 M formic acid/3 M ammonium formate and rinsed twice with H₂O and stored at 4° C. in water.

### Example 261

### High Throughput Functional Screening: FLIPR™

Subsequently, a functional based assay was used to confirm the lead hits, referred to as FLIPR™ (the Fluorometric Imaging Plate Reader) and FDSS6000™ (Functional Drug Screening System). This assay utilized a non-endogenous, constitutively active version of the MCH receptor.

The FLIPR and FDSS assays are able to detect intracellular Ca²⁺ concentration in cells, which can be utilized to assess receptor activation and determine whether a candidate compound is an, for example, antagonist, inverse agonist or agonist to a Gq-coupled receptor. The concentration of free Ca²⁺ in the cytosol of any cell is extremely low, whereas its concentration in the extracellular fluid and endoplasmic reticulum (ER) is very high. Thus, there is a large gradient tending to drive Ca²⁺ into the cytosol across both the plasma membrane and ER. The FLIPR™ and FDSS6000™ systems (Molecular Devices Corporation, HAMAMATSU Photonics K.K.) are designed to perform functional cell-based assays, such as the measurement of intracellular calcium for high-throughput screening. The measurement of fluorescent is associated with calcium release upon activation of the Gq-coupled receptors. Gi or Go coupled receptors are not as easily monitored through the FLIPR™ and FDSS6000™ systems because these G proteins do not couple with calcium signal pathways.

Fluorometric Imaging Plate Reader system was used to allow for rapid, kinetic measurements of intracellular fluorescence in 96 well microplates (or 384 well microplates). Simultaneous measurements of fluorescence in all wells can be made by FLIPR or FDSS6000™ every second with high sensitivity and precision. These systems are ideal for measuring cell-based functional assays such as monitoring the intracellular calcium fluxes that occur within seconds after activation of the Gq coupled receptor.

Briefly, the cells are seeded into 96 well at 5.5×10⁴ cells/well with complete culture media (Dulbecco's Modified Eagle Medium with 10% fetal bovine serum, 2 mM L-glutamine, 1 mM sodium pyruvate and 0.5 mg/mL G418, pH 7.4) for the assay next day. On the day of assay, the media is removed and the cells are incubated with 100 μl of loading buffer (4 μM Fluo4-AM in complete culture media containing 2.5 mM Probenicid, 0.5 mg/ml and 0.2% bovine serum albumin) in 5% CO₂ incubator at 37° C. for 1 hr. The loading buffer is removed, and the cells are washed with wash buffer (Hank's Balanced Salt Solution containing 2.5 mM Probenicid, 20 mM HEPES, 0.5 mg/mL and 0.2% bovine serum albumin, pH 7.4). One hundred fifty μl of wash buffer containing various concentrations of test compound is added to the cells, and the cells are incubated in 5% CO₂ incubator at 37° C. for 30 min. Fifty μl of wash buffer containing various concentration of MCH are added to each well, and transient changes in [Ca²⁺]i evoked by MCH are monitored using the FLIPR™ or FDSS in 96 well plates at Ex. 488 nm and Em. 530 nm for 290 second. When antagonist activity of compound is tested, 50 nM of MCH is used.

Use of FLIPR™ and FDSS6000™ can be accomplished by following manufacturer's instruction (Molecular Device Corporation and HAMAMATSU Photonics K.K.).

Representative examples are shown below.

| Compound No. | IC₅₀ (nM) |
|---|---|
| Example 4 | 1.0 |
| Example 5 | 1.9 |

The results were shown on the below table in accordance with the classification as defined below.
Class 1: The value of percent of control at 10⁻⁷ M was less than 40% or the value of IC₅₀ was less than 50 nM.

Class 1: The value of percent of control at 10⁻⁷ M was from 40% to 60% or the value of IC₅₀ was from 50 nM to 200 nM.

| Ex. No. | class |
|---|---|
| 2 | 2 |
| 3 | 2 |
| 4 | 1 |
| 5 | 1 |
| 6 | 1 |
| 7 | 1 |
| 8 | 1 |
| 9 | 1 |
| 10 | 1 |
| 11 | 1 |
| 12 | 1 |
| 13 | 1 |
| 14 | 1 |
| 15 | 1 |
| 16 | 1 |
| 17 | 2 |
| 18 | 2 |
| 19 | 2 |
| 20 | 2 |
| 25 | 2 |
| 26 | 2 |
| 27 | 2 |
| 28 | 1 |
| 29 | 1 |
| 32 | 2 |
| 33 | 1 |
| 34 | 1 |
| 35 | 2 |
| 36 | 1 |
| 37 | 1 |
| 38 | 1 |
| 39 | 1 |
| 40 | 1 |
| 43 | 1 |
| 49 | 2 |
| 50 | 1 |
| 51 | 1 |
| 52 | 1 |
| 53 | 2 |
| 146 | 1 |
| 147 | 1 |
| 166 | 2 |
| 168 | 2 |
| 171 | 1 |
| 177 | 2 |
| 181 | 1 |
| 185 | 1 |
| 186 | 1 |
| 190 | 1 |
| 192 | 1 |
| 203 | 2 |
| 209 | 2 |

### Example 262

### Receptor Binding Assay

In addition to the methods described herein, another means for evaluating a test compound is by determining binding affinities to the MCH receptor. This type of assay generally requires a radiolabelled ligand to the MCH receptor. Absent the use of known ligands for the MCH receptor and radiolabels thereof, compounds of Formula (I) can be labelled with a radioisotope and used in an assay for evaluating the affinity of a test compound to the MCH receptor.

A radiolabelled MCH compound of Formula (I) can be used in a screening assay to identify/evaluate compounds. In general terms, a newly synthesized or identified compound (i.e., test compound) can be evaluated for its ability to reduce binding of the “radiolabelled compound of Formula (I)” to the MCH receptor. Accordingly, the ability to compete with the “radio-labelled compound of Formula (I)” or Radiolabelled MCH Ligand for the binding to the MCH receptor directly correlates to its binding affinity of the test compound to the MCH receptor.

Assay Protocol for Determining Receptor Binding for MCH:

A. MCH Receptor Preparation

293 cells (human kidney, ATCC), transiently transfected with 10 μg human MCH receptor and 60 μl Lipofectamine (per 15-cm dish), are grown in the dish for 24 hours (75% confluency) with a media change and removed with 10 mL/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-mL Dounce homogenizer and again centrifuged. After removing the supernatant, the pellets can be stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 μl of appropriately diluted membranes (diluted in assay buffer containing 50 mM Tris HCl (pH 7.4), 10 mM MgCl₂, and 1 mM EDTA; 5-50 μg protein) is added to 96-well polyproylene microtiter plates followed by addition of 100 μl of assay buffer and 50 μl of Radiolabelled MCH Ligand. For nonspecific binding, 50 μl of assay buffer is added instead of 100 μl and an additional 50 μl of 10 μM cold MCH is added before 50 μl of Radiolabelled MCH Ligand is added. Plates are then incubated at room temperature for 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plate are sealed, 50 μl of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted test compound is added to appropriate wells followed by addition of 50 μl of Radiolabelled MCH Ligand.

C. Calculations

The test compounds are initially assayed at 1 and 0.1 μM and then at a range of concentrations chosen such that the middle dose would cause about 50% inhibition of a Radiolabelled MCH Ligand binding (i.e., IC₅₀). Specific binding in the absence of test compound (B_{O}) is the difference of total binding (B_{T}) minus non-specific binding (NSB) and similarly specific binding (in the presence of test compound) (B) is the difference of displacement binding (B_{D}) minus non-specific binding (NSB). IC₅₀ is determined from an inhibition response curve, logit-log plot of % B/B_{O} vs concentration of test compound.

Kᵢ is calculated by the Cheng and Prustoff transformation:
Kᵢ=IC₅₀/(1+[L]/K_{D})

wherein [L] is the concentration of a Radiolabelled MCH Ligand used in the assay and K_{D} is the dissociation constant of a Radiolabelled MCH Ligand determined independently under the same binding conditions.

It is intended that each of the patents, applications, printed publications, and other published documents mentioned or referred to in this specification be herein incorporated by reference in their entirety.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

1. A compound of Formula (I): 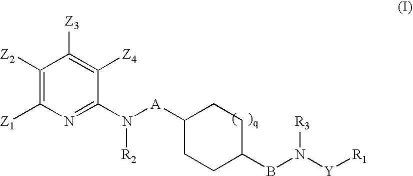
R₁ is selected from the group consisting of:
(i) C₁₋₁₀ alkyl, and
C₁₋₁₀ alkyl substituted by substituent(s) independently selected from the group consisting of:
halogen,
oxo,
C₁₋₅ alkoxy,
C₁₋₅ alkoxy substituted by carbocyclic aryl,
C₁₋₅ alkylcarbonyloxy,
carbocyclic aryloxy,
carbocyclic aryloxy substituted by substituent(s) independently selected from the group consisting of:
halogen,
nitro,
C₁₋₅ alkyl, and
C₁₋₅ alkyl substituted by oxo,
heterocyclyloxy,
heterocyclyloxy substituted by C₁₋₅ alkyl,
C₁₋₅ alkoxycarbonyl,
C₁₋₅ alkoxycarbonyl substituted by carbocyclic aryl,
mono-carbocyclic arylamino,
mono-carbocyclic arylamino substituted by hydroxy,
di-carbocyclic arylamino,
di-carbocyclic arylamino substituted by hydroxy,
C₁₋₅ alkylcarbonylamino,
heterocyclyl carbonylamino,
carbocyclic arylsulfonylamino,
carbocyclic arylsulfonylamino substituted by nitro,
carbocyclic arylsulfonylamino substituted by C₁₋₅ alkyl,
C₁₋₅ alkylthio,
C₁₋₅ alkylthio substituted by carbocyclic aryl,
carbocyclic arylthio,
carbocyclic arylthio substituted by halogen,
carbocyclic arylthio substituted by C₁₋₅ alkyl,
carbocyclic arylsulfonyl,
carbocyclic arylsulfonyl substituted by halogen,
heterocyclylthio,
heterocyclylthio substituted by C₁₋₅ alkyl,
C₃₋₆ cycloalkyl,
C₃₋₆ cycloalkenyl,
carbocyclyl,
carbocyclyl substituted by C₁₋₅ alkoxy,
carbocyclic aryl,
carbocyclic aryl substituted by substituent(s) independently selected from the group consisting of:
halogen,
hydroxy,
nitro,
C₁₋₅ alkyl,
C₁₋₅ alkyl substituted by oxo,
C₁₋₅ alkyl substituted by carbocyclic aryl,
C₁₋₅ alkyl substituted by heterocyclyl,
C₁₋₅ alkoxy,
C₁₋₅ alkoxy substituted by halogen,
C₁₋₅ alkoxy substituted by carbocyclic aryl,
carbocyclic aryloxy,
mono-carbocyclic arylamino,
mono-carbocyclic arylamino substituted by halogen,
di-carbocyclic arylamino,
di-carbocyclic arylamino substituted by halogen,
mono-carbocyclic arylaminocarbonyl,
mono-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
di-carbocyclic arylaminocarbonyl,
di-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
mercapto,
C₁₋₅ alkylthio,
C₁₋₅ alkylthio substituted by halogen,
C₁₋₅ alkylsulfonyl,
carbocyclic aryl, and
heterocyclyl,
heterocyclyl, and
heterocyclyl substituted by substituent(s) independently selected from the group consisting of:
C₁₋₅ alkyl,
C₁₋₅ alkoxy,
C₁₋₅ alkoxy substituted by carbocyclic aryl,
carbocyclic aryl, and
carbocyclic aryl substituted by halogen,
(ii) C₂₋₅ alkenyl, and
C₂₋₅ alkenyl substituted by substituent(s) independently selected from the group consisting of:
oxo,
carbocyclic aryl,
carbocyclic aryl substituted by substituent(s) independently selected from the group consisting of:
halogen,
nitro,
C₁₋₅ alkyl,
C₁₋₅ alkyl substituted by halogen,
C₁₋₅ alkoxy, and
C₁₋₅ alkoxy substituted by halogen,
(iii) C₃₋₆ cycloalkyl, and
C₃₋₆ cycloalkyl substituted by substituent(s) independently selected from the group consisting of:
C₁₋₅ alkyl,
C₁₋₅ alkyl substituted by oxo,
C₁₋₅ alkyl substituted by carbocyclic aryl,
carbocyclic arylcarbonylamino, and
carbocyclic aryl, and
carbocyclic aryl by halogen,
(iv) carbocyclyl, and
carbocyclyl substituted by nitro,
carbocyclic aryl, and
carbocyclic aryl substituted by substituent(s) independently selected from the group consisting of:
halogen,
hydroxy,
cyano,
nitro,
C₁₋₉ alkyl,
C₁₋₉ alkyl substituted by substituent(s) independently selected from the group consisting of:
oxo,
halogen,
carbocyclic aryloxy,
mono-carbocyclic arylaminocarbonyl,
di-carbocyclic arylaminocarbonyl,
mono-carbocyclic arylaminocarbonyl substituted by C₁₋₅ alkoxy,
di-carbocyclic arylaminocarbonyl substituted by C₁₋₅ alkoxy,
carbocyclic aryl,
carbocyclic aryl substituted by substituent(s) independently selected from the group consisting of:
halogen,
C₁₋₅ alkyl, and
C₁₋₅ alkyl substituted by halogen,
heterocyclyl, and
heterocyclyl substituted by C₁₋₅ alkyl,
C₁₋₇ alkoxy,
C₁₋₇ alkoxy substituted by halogen,
C₁₋₇ alkoxy substituted by carbocyclic aryl,
C₁₋₅ alkylcarbonyloxy,
carbocyclic aryloxy,
carbocyclic aryloxy substituted by C₁₋₅ alkoxy,
C₁₋₅ alkoxycarbonyl,
mono-C₁₋₅ alkylaminocarbonyl,
di-C₁₋₅ alkylaminocarbonyl,
mono-C₁₋₅ alkylaminocarbonyl substituted by carbocyclic aryl,
di-C₁₋₅ alkylaminocarbonyl substituted by carbocyclic aryl,
mono-carbocyclic arylaminocarbonyl,
di-carbocyclic arylaminocarbonyl,
mono-carbocyclic arylaminocarbonyl substituted by C₁₋₅ alkyl,
di-carbocyclic arylaminocarbonyl substituted by C₁₋₅ alkyl,
mono-C₁₋₅ alkylamino,
di-C₁₋₅ alkylamino,
carbocyclic arylsulfonylamino,
carbocyclic arylsulfonylamino substituted by C₁₋₅ alkyl,
C₁₋₅ alkylthio,
C₁₋₅ alkylthio substituted by halogen,
carbocyclic arylthio,
carbocyclic arylthio substituted by cyano,
C₁₋₅ alkylsulfonyl,
mono-C₁₋₅ alkylaminosulfonyl,
di-C₁₋₅ alkylaminosulfonyl,
carbocyclic aryl,
carbocyclic aryl substituted by substituent(s) independently selected from the group consisting of:
C₁₋₇ alkyl, and
C₁₋₇ alkyl substituted by halogen,
heterocyclyl,
heterocyclyl, and
heterocyclyl substituted by substituent(s) independently selected from the group consisting of:
halogen,
nitro,
C₁₋₅ alkyl,
C₁₋₅ alkyl substituted by substituent(s) independently selected from the group consisting of:
halogen,
oxo,
carbocyclic aryl,
carbocyclic aryl substituted by halogen, and
heterocyclyl,
C₁₋₅ alkoxy,
carbocyclic aryloxy,
carbocyclic aryloxy substituted by C₁₋₅ alkyl,
C₁₋₅ alkylthio,
carbocyclic arylthio,
C₁₋₅ alkylsulfonyl,
carbocyclic arylsulfonyl,
carbocyclic arylsulfonyl substituted by halogen,
carbocyclic arylsulfonyl substituted by C₁₋₅ alkyl,
carbocyclic aryl,
carbocyclic aryl substituted by substituent(s) independently selected from the group consisting of:
halogen,
nitro, and
C₁₋₅ alkyl,
heterocyclyl, and
heterocyclyl substituted by substituent(s) independently selected from the group consisting of:
C₁₋₅ alkyl, and
C₁₋₅ alkyl substituted by halogen;
R₂ and R₃ are each independently hydrogen or C₁₋₅ alkyl; and A and B are each independently a single bond, —CH₂—, or —(CH₂)₂—;
Z₁, Z₂, Z₃, and Z₄ are each independently hydrogen, halogen, cyano, nitro, carboxy, carbamoyl, C₁₋₅ alkyl, C₁₋₅ alkyl substituted by halogen, C₁₋₅ alkyl substituted by hydroxy, C₁₋₅ alkoxy, C₁₋₅ alkoxy substituted by halogen, C₁₋₅ alkoxy substituted by hydroxy, —CO₂R₄ₐ, —C(O)N(R₄ₐ)(R_{4b}), —N(R₄ₐ)(R_{4b}), or heterocyclyl; wherein R₄ₐ and R_{4b} are each independently hydrogen, C₁₋₅ alkyl, or C₁₋₅ alkyl substituted by substituent(s) independently selected from the group consisting of:
halogen,
hydroxy,
carboxy,
carbamoyl,
C₁₋₅ alkoxy,
amino,
C₃₋₆ cycloalkyl,
carbocyclic aryl,
carbocyclic aryl substituted by substituent(s) independently selected from the group consisting of:
halogen,
C₁₋₅ alkyl,
C₁₋₅ alkyl substituted by halogen,
C₁₋₅ alkoxy,
C₁₋₅ alkoxy substituted by halogen, and
—SO₂NH₂,
heterocyclyl, and
C₃₋₆ cycloalkyl, carbocyclic aryl, carbocyclic aryl substituted by substituent(s) independently selected from the group consisting of:
halogen,
C₁₋₅ alkyl, and
C₁₋₅ alkoxy;
or
Z₁ and Z₂ are bonded to each other to form a ring and -Z₁-Z₂- is —(CH₂)ₘ— or —(CH₂)ₙ—CH═CH—(CH₂)ₒ—; wherein one —CH₂— group of -Z₁-Z₂- can optionally be replaced by C(O), NR₅, O, S, S(O), or S(O)₂; wherein m is 2, 3, 4, 5, or 6; n and o are each independently 0, 1, 2, 3, or 4 provided that n+o=0, 1, 2, 3, or 4; and R₅ is hydrogen or C₁₋₅ alkyl;
Y is —S(O)₂—, —C(O)—, —C(O)NR₆—, —C(S)NR₆—, —C(O)O—, or —(CH₂)ₚ—; wherein R₆ is hydrogen or C₁₋₅ alkyl; p is 0, 1, 2, 3, 4, or 5; and
q is 0 or 1;
wherein carbocyclic aryl is phenyl, naphthyl, or anthranyl;
carbocyclyl is 1-oxo-indanyl, 9H-fluorenyl, 9-oxo-fluorenyl, anthraquinonyl, C-fluoren-9-ylidene, or indanyl;
heterocyclyl is 1,2,3-thiadiazolyl, 1,2,3-triazolyl, 1,3-dioxo-isoindolyl, 1H-indolyl, 1H-pyrrolyl, 2,3-dihydro-benzo[1,4]dioxinyl, 2,3-dihydro-benzofuryl, 2H-benzopyranyl, 2-oxo-benzopyranyl, 2-oxo-pyrrolidinyl, 4-oxo-3,4-dihydro-phthalazinyl, 4-oxo-benzopyranyl, 9H-xanthenyl, benzimidazolyl, benzo[1,3]dioxolyl, benzo[2,1,3]oxadiazolyl, benzo[1,2,5]oxadiazolyl, benzo[b]thienyl, benzofuryl, benzothiazolyl, cinnolyl, furyl, imidazolyl, isoxazolyl, morpholino, morpholinyl, oxazolyl, piperazyl, piperidyl, pyrazolyl, pyrazinyl, pyridyl, pyrimidyl, pyrrolidinyl, quinolyl, quinoxalyl, thiazolidyl, thiazolyl, or thienyl; and
halogen is fluoro, chloro, bromo, or iodo;
or a pharmaceutically acceptable salt, hydrate, or solvate thereof.

2. The compound according to claim 1 wherein R₁ is selected from the group consisting of:
(i) C₁₋₁₀ alkyl substituted by substituent(s) independently selected from the group consisting of:
oxo,
C₁₋₅ alkoxy substituted by carbocyclic aryl,
C₁₋₅ alkylcarbonyloxy,
carbocyclic aryloxy,
carbocyclic aryloxy substituted by substituent(s) independently selected from the group consisting of:
halogen,
nitro,
C₁₋₅ alkyl, and
C₁₋₅ alkyl substituted by oxo,
heterocyclyloxy,
heterocyclyloxy substituted by C₁₋₅ alkyl,
C₁₋₅ alkoxycarbonyl,
C₁₋₅ alkoxycarbonyl substituted by carbocyclic aryl,
mono-carbocyclic arylamino,
mono-carbocyclic arylamino substituted by hydroxy,
di-carbocyclic arylamino,
di-carbocyclic arylamino substituted by hydroxy,
C₁₋₅ alkylcarbonylamino,
heterocyclyl carbonylamino,
carbocyclic arylsulfonylamino,
carbocyclic arylsulfonylamino substituted by nitro,
carbocyclic arylsulfonylamino substituted by C₁₋₅ alkyl,
C₁₋₅ alkylthio,
C₁₋₅ alkylthio substituted by carbocyclic aryl,
carbocyclic arylthio,
carbocyclic arylthio substituted by halogen,
carbocyclic arylthio substituted by C₁₋₅ alkyl,
carbocyclic arylsulfonyl,
carbocyclic arylsulfonyl substituted by halogen,
heterocyclylthio,
heterocyclylthio substituted by C₁₋₅ alkyl,
C₃₋₆ cycloalkyl,
C₃₋₆ cycloalkenyl,
carbocyclyl,
carbocyclyl substituted by C₁₋₅ alkoxy,
carbocyclic aryl,
carbocyclic aryl substituted by substituent(s) independently selected from the group consisting of:
halogen,
hydroxy,
nitro,
C₁₋₅ alkyl,
C₁₋₅ alkyl substituted by oxo,
C₁₋₅ alkyl substituted by carbocyclic aryl,
C₁₋₅ alkyl substituted by heterocyclyl,
C₁₋₅ alkoxy,
C₁₋₅ alkoxy substituted by halogen,
C₁₋₅ alkoxy substituted by carbocyclic aryl,
carbocyclic aryloxy,
mono-carbocyclic arylamino,
mono-carbocyclic arylamino substituted by halogen,
di-carbocyclic arylamino,
di-carbocyclic arylamino substituted by halogen,
mono-carbocyclic arylaminocarbonyl,
mono-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
di-carbocyclic arylaminocarbonyl,
di-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
mercapto,
C₁₋₅ alkylthio,
C₁₋₅ alkylthio substituted by halogen,
C₁₋₅ alkylsulfonyl,
carbocyclic aryl, and
heterocyclyl,
heterocyclyl, and
heterocyclyl substituted by substituent(s) independently selected from the group consisting of:
C₁₋₅ alkyl,
C₁₋₅ alkoxy,
C₁₋₅ alkoxy substituted by carbocyclic aryl,
carbocyclic aryl, and
carbocyclic aryl substituted by halogen,
(ii) C₂₋₅ alkenyl substituted by substituent(s) independently selected from the group consisting of:
oxo,
carbocyclic aryl,
carbocyclic aryl substituted by substituent(s) independently selected from the group consisting of:
halogen,
nitro,
C₁₋₅ alkyl,
C₁₋₅ alkyl substituted by halogen,
C₁₋₅ alkoxy, and
C₁₋₅ alkoxy substituted by halogen,
(iii) C₃₋₆ cycloalkyl substituted by substituent(s) independently selected from the group consisting of:
C₁₋₅ alkyl,
C₁₋₅ alkyl substituted by oxo,
C₁₋₅ alkyl substituted by carbocyclic aryl,
carbocyclic arylcarbonylamino, and
carbocyclic aryl, and
carbocyclic aryl by halogen,
(iv) carbocyclyl, and
carbocyclyl substituted by nitro,
carbocyclic aryl, and
carbocyclic aryl substituted by substituent(s) independently selected from the group consisting of:
halogen,
hydroxy,
cyano,
nitro,
C₁₋₉ alkyl,
C₁₋₉ alkyl substituted by substituent(s) independently selected from the group consisting of:
oxo,
halogen,
carbocyclic aryloxy,
mono-carbocyclic arylaminocarbonyl,
di-carbocyclic arylaminocarbonyl,
mono-carbocyclic arylaminocarbonyl substituted by C₁₋₅ alkoxy,
di-carbocyclic arylaminocarbonyl substituted by C₁₋₅ alkoxy,
carbocyclic aryl,
carbocyclic aryl substituted by substituent(s) independently selected from the group consisting of:
halogen,
C₁₋₅ alkyl, and
C₁₋₅ alkyl substituted by halogen,
heterocyclyl, and
heterocyclyl substituted by C₁₋₅ alkyl,
C₁₋₇ alkoxy,
C₁₋₇ alkoxy substituted by halogen,
C₁₋₇ alkoxy substituted by carbocyclic aryl,
C₁₋₅ alkylcarbonyloxy,
carbocyclic aryloxy,
carbocyclic aryloxy substituted by C₁₋₅ alkoxy,
C₁₋₅ alkoxycarbonyl,
mono-C₁₋₅ alkylaminocarbonyl,
di-C₁₋₅ alkylaminocarbonyl,
mono-C₁₋₅ alkylaminocarbonyl substituted by carbocyclic aryl,
di-C₁₋₅ alkylaminocarbonyl substituted by carbocyclic aryl,
mono-carbocyclic arylaminocarbonyl,
di-carbocyclic arylaminocarbonyl,
mono-carbocyclic arylaminocarbonyl substituted by C₁₋₅ alkyl,
di-carbocyclic arylaminocarbonyl substituted by C₁₋₅ alkyl,
mono-C₁₋₅ alkylamino,
di-C₁₋₅ alkylamino,
carbocyclic arylsulfonylamino,
carbocyclic arylsulfonylamino substituted by C₁₋₅ alkyl,
C₁₋₅ alkylthio,
C₁₋₅ alkylthio substituted by halogen,
carbocyclic arylthio,
carbocyclic arylthio substituted by cyano,
C₁₋₅ alkylsulfonyl,
mono-C₁₋₅ alkylaminosulfonyl,
di-C₁₋₅ alkylaminosulfonyl,
carbocyclic aryl,
carbocyclic aryl substituted by substituent(s) independently selected from the group consisting of:
C₁₋₇ alkyl, and
C₁₋₇ alkyl substituted by halogen,
heterocyclyl,
heterocyclyl, and
heterocyclyl substituted by substituent(s) independently selected from the group consisting of:
halogen,
nitro,
C₁₋₅ alkyl,
C₁₋₅ alkyl substituted by substituent(s) independently selected from the group consisting of:
halogen,
oxo,
carbocyclic aryl,
carbocyclic aryl substituted by halogen, and
heterocyclyl,
C₁₋₅ alkoxy,
carbocyclic aryloxy,
carbocyclic aryloxy substituted by C₁₋₅ alkyl,
C₁₋₅ alkylthio,
carbocyclic arylthio,
C₁₋₅ alkylsulfonyl,
carbocyclic arylsulfonyl,
carbocyclic arylsulfonyl substituted by halogen,
carbocyclic arylsulfonyl substituted by C₁₋₅ alkyl,
carbocyclic aryl,
carbocyclic aryl substituted by substituent(s) independently selected from the group consisting of:
halogen,
nitro, and
C₁₋₅ alkyl,
heterocyclyl, and
heterocyclyl substituted by substituent(s) independently selected from the group consisting of:
C₁₋₅ alkyl, and
C₁₋₅ alkyl substituted by halogen;
Z₁, Z₂, Z₃, and Z₄ are each independently hydrogen, halogen, cyano, nitro, carboxy, carbamoyl, C₁₋₅ alkyl, C₁₋₅ alkyl substituted by halogen, C₁₋₅ alkyl substituted by hydroxy, C₁₋₅ alkoxy, C₁₋₅ alkoxy substituted by halogen, C₁₋₅ alkoxy substituted by hydroxy, —CO₂R₄ₐ, —C(O)N(R₄ₐ)(R_{4b}), —N(R₄ₐ)(R_{4b}), or heterocyclyl; wherein R₄ₐ and R_{4b} are each independently hydrogen, C₁₋₅ alkyl, or C₁₋₅ alkyl substituted by substituent(s) independently selected from the group consisting of:
halogen,
hydroxy,
carboxy,
carbamoyl,
C₁₋₅ alkoxy,
amino,
C₃₋₆ cycloalkyl,
carbocyclic aryl,
carbocyclic aryl substituted by substituent(s) independently selected from the group consisting of:
halogen,
C₁₋₅ alkyl,
C₁₋₅ alkyl substituted by halogen,
C₁₋₅ alkoxy,
C₁₋₅ alkoxy substituted by halogen, and
—SO₂NH₂,
heterocyclyl, and
C₃₋₆ cycloalkyl, carbocyclic aryl, carbocyclic aryl substituted by substituent(s) independently selected from the group consisting of:
halogen,
C₁₋₅ alkyl, and
C₁₋₅ alkoxy;
wherein Z₂ is not —C(O)N(R₄ₐ)(R_{4b});
or
Z₁ and Z₂ are bonded to each other to form a ring and -Z₁-Z₂- is —(CH₂)ₘ— or —(CH₂)ₙ—CH═CH—(CH₂)ₒ—; wherein one —CH₂— group of -Z₁-Z₂- can optionally be replaced by C(O), NR₅, O, S, S(O), or S(O)₂; wherein m is 2, 3, 4, 5, or 6; n and o are each independently 0, 1, 2, 3, or 4 provided that n+o=0, 1, 2, 3, or 4; and R₅ is hydrogen or C₁₋₅ alkyl;
Y is —S(O)₂—, —C(O)—, —C(S)NR₆—, —C(O)O—, or —(CH₂)ₚ—; wherein R₆ is hydrogen or C₁₋₅ alkyl; p is 0, 1, 2, 3, 4, or 5; and
q is 0 or 1;
wherein carbocyclic aryl is phenyl, naphthyl, or anthranyl;
carbocyclyl is 1-oxo-indanyl, 9H-fluorenyl, 9-oxo-fluorenyl, anthraquinonyl, C-fluoren-9-ylidene, or indanyl;
heterocyclyl is 1,2,3-thiadiazolyl, 1,2,3-triazolyl, 1,3-dioxo-isoindolyl, 1H-indolyl, 1H-pyrrolyl, 2,3-dihydro-benzo[1,4]dioxinyl, 2,3-dihydro-benzofuryl, 2H-benzopyranyl, 2-oxo-benzopyranyl, 2-oxo-pyrrolidinyl, 4-oxo-3,4-dihydro-phthalazinyl, 4-oxo-benzopyranyl, 9H-xanthenyl, benzimidazolyl, benzo[1,3]dioxolyl, benzo[2,1,3]oxadiazolyl, benzo[1,2,5]oxadiazolyl, benzo[b]thienyl, benzofuryl, benzothiazolyl, cinnolyl, furyl, imidazolyl, isoxazolyl, morpholino, morpholinyl, oxazolyl, piperazyl, piperidyl, pyrazolyl, pyrazinyl, pyridyl, pyrimidyl, pyrrolidinyl, quinolyl, quinoxalyl, thiazolidyl, thiazolyl, or thienyl; and
halogen is fluoro, chloro, bromo, or iodo;
or a pharmaceutically acceptable salt, hydrate, or solvate thereof.

3. The compound according to claim 2 wherein Formula (I) is Formula (Ia): 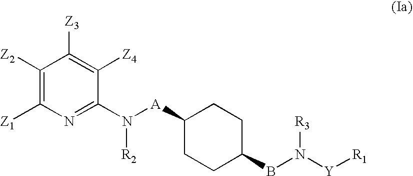
or a pharmaceutically acceptable salt, hydrate, or solvate thereof.

4. The compound according to claim 3 wherein R₁ is selected from the group consisting of:
(i) C₁₋₁₀ alkyl substituted by substituent(s) independently selected from the group consisting of:
oxo,
C₁₋₅ alkoxy substituted by carbocyclic aryl,
carbocyclic aryloxy,
carbocyclic aryloxy substituted by substituent(s) independently selected from the group consisting of:
halogen,
nitro,
C₁₋₅ alkyl, and
C₁₋₅ alkyl substituted by oxo,
C₁₋₅ alkoxycarbonyl,
C₁₋₅ alkoxycarbonyl substituted by carbocyclic aryl,
mono-carbocyclic arylamino,
di-carbocyclic arylamino,
heterocyclyl carbonylamino,
carbocyclic arylsulfonylamino,
carbocyclic arylsulfonylamino substituted by nitro,
carbocyclic arylsulfonylamino substituted by C₁₋₅ alkyl,
C₁₋₅ alkylthio,
C₁₋₅ alkylthio substituted by carbocyclic aryl,
carbocyclic arylthio,
carbocyclic arylthio substituted by halogen,
carbocyclic arylthio substituted by C₁₋₅ alkyl,
carbocyclic arylsulfonyl,
carbocyclic arylsulfonyl substituted by halogen,
heterocyclylthio,
heterocyclylthio substituted by C₁₋₅ alkyl,
C₃₋₆ cycloalkyl,
C₃₋₆ cycloalkenyl,
carbocyclyl,
carbocyclyl substituted by C₁₋₅ alkoxy,
carbocyclic aryl,
carbocyclic aryl substituted by substituent(s) independently selected from the group consisting of:
halogen,
nitro,
C₁₋₅ alkyl,
C₁₋₅ alkyl substituted by oxo,
C₁₋₅ alkyl substituted by carbocyclic aryl,
C₁₋₅ alkyl substituted by heterocyclyl,
C₁₋₅ alkoxy,
C₁₋₅ alkoxy substituted by halogen,
C₁₋₅ alkoxy substituted by carbocyclic aryl,
carbocyclic aryloxy,
mono-carbocyclic arylaminocarbonyl,
mono-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
di-carbocyclic arylaminocarbonyl,
di-carbocyclic arylaminocarbonyl substituted by substituent(s) selected from the group consisting of:
halogen,
C₁₋₅ alkyl,
C₁₋₅ alkoxy, and
C₁₋₅ alkoxy substituted by halogen,
C₁₋₅ alkylsulfonyl,
carbocyclic aryl, and
heterocyclyl,
heterocyclyl, and
heterocyclyl substituted by substituent(s) independently selected from the group consisting of:
C₁₋₅ alkyl,
C₁₋₅ alkoxy,
C₁₋₅ alkoxy substituted by carbocyclic aryl,
carbocyclic aryl, and
carbocyclic aryl substituted by halogen,
(ii) C₂₋₅ alkenyl substituted by substituent(s) independently selected from the group consisting of:
oxo,
carbocyclic aryl,
carbocyclic aryl substituted by substituent(s) independently selected from the group consisting of:
halogen,
nitro,
C₁₋₅ alkyl, and
C₁₋₅ alkyl substituted by halogen,
(iii) C₃₋₆ cycloalkyl substituted by substituent(s) independently selected from the group consisting of:
C₁₋₅ alkyl,
C₁₋₅ alkyl substituted by oxo,
C₁₋₅ alkyl substituted by carbocyclic aryl,
carbocyclic arylcarbonylamino, and
carbocyclic aryl, and
carbocyclic aryl by halogen,
(iv) carbocyclyl, and
carbocyclyl substituted by nitro,
carbocyclic aryl, and
carbocyclic aryl substituted by substituent(s) independently selected from the group consisting of:
halogen,
cyano,
nitro,
C₁₋₉ alkyl,
C₁₋₉ alkyl substituted by substituent(s) independently selected from the group consisting of:
oxo,
halogen,
carbocyclic aryloxy,
mono-carbocyclic arylaminocarbonyl,
di-carbocyclic arylaminocarbonyl,
mono-carbocyclic arylaminocarbonyl substituted by C₁₋₅ alkoxy,
di-carbocyclic arylaminocarbonyl substituted by C₁₋₅ alkoxy,
carbocyclic aryl,
carbocyclic aryl substituted by substituent(s) independently selected from the group consisting of:
halogen,
C₁₋₅ alkyl, and
C₁₋₅ alkyl substituted by halogen,
heterocyclyl, and
heterocyclyl substituted by C₁₋₅ alkyl,
C₁₋₇ alkoxy,
C₁₋₇ alkoxy substituted by halogen,
C₁₋₇ alkoxy substituted by carbocyclic aryl,
carbocyclic aryloxy,
carbocyclic aryloxy substituted by C₁₋₅ alkoxy,
C₁₋₅ alkoxycarbonyl,
mono-C₁₋₅ alkylaminocarbonyl,
di-C₁₋₅ alkylaminocarbonyl,
mono-C₁₋₅ alkylaminocarbonyl substituted by carbocyclic aryl,
di-C₁₋₅ alkylaminocarbonyl substituted by carbocyclic aryl,
mono-carbocyclic arylaminocarbonyl,
di-carbocyclic arylaminocarbonyl,
mono-carbocyclic arylaminocarbonyl substituted by C₁₋₅ alkyl,
di-carbocyclic arylaminocarbonyl substituted by C₁₋₅ alkyl,
mono-C₁₋₅ alkylamino,
di-C₁₋₅ alkylamino,
carbocyclic arylsulfonylamino,
carbocyclic arylsulfonylamino substituted by C₁₋₅ alkyl,
C₁₋₅ alkylthio,
C₁₋₅ alkylthio substituted by halogen,
carbocyclic arylthio,
carbocyclic arylthio substituted by cyano,
C₁₋₅ alkylsulfonyl,
mono-C₁₋₅ alkylaminosulfonyl,
di-C₁₋₅ alkylaminosulfonyl,
carbocyclic aryl,
carbocyclic aryl substituted by substituent(s) independently selected from the group consisting of:
C₁₋₇ alkyl, and
C₁₋₇ alkyl substituted by halogen,
heterocyclyl,
heterocyclyl, and
heterocyclyl substituted by substituent(s) independently selected from the group consisting of:
halogen,
nitro,
C₁₋₅ alkyl,
C₁₋₅ alkyl substituted by substituent(s) independently selected from the group consisting of:
halogen,
oxo, and
heterocyclyl,
C₁₋₅ alkoxy,
carbocyclic aryloxy,
C₁₋₅ alkylthio,
C₁₋₅ alkylsulfonyl,
carbocyclic arylsulfonyl,
carbocyclic arylsulfonyl substituted by halogen,
carbocyclic arylsulfonyl substituted by C₁₋₅ alkyl,
carbocyclic aryl;
R₂ and R₃ are each hydrogen; and A and B are each independently a single bond or —CH₂—, provided that A is not —CH₂— when B is —CH₂—;
Z₁ and Z₂ are each independently hydrogen, halogen, C₁₋₅ alkyl, or —N(R₄ₐ)(R_{4b}); Z₃ is hydrogen, cyano, nitro, carbamoyl, C₁₋₅ alkyl, C₁₋₅ alkyl substituted by hydroxy, C₁₋₅ alkoxy, —C(O)N(R₄ₐ)(R_{4b}), —N(R₄ₐ)(R_{4b}), morpholinyl, pyrrolidinyl, or imidazolyl; wherein R₄ₐ and R_{4b} are each independently hydrogen, C₁₋₅ alkyl, or C₁₋₅ alkyl substituted by carbocyclic aryl; Z₄ is hydrogen, halogen, or C₁₋₅ alkyl;
or
Z₁ and Z₂ are bonded to each other to form a ring and -Z₁-Z₂- is —(CH₂)ₘ—; wherein m is 3 or 4; and
Y is —S(O)₂—, —C(O)—, —C(S)NH—, —C(O)O—, or —CH₂—;
wherein carbocyclic aryl is phenyl, naphthyl, or anthranyl;
carbocyclyl is 1-oxo-indanyl, 9H-fluorenyl, 9-oxo-fluorenyl, anthraquinonyl, C-fluoren-9-ylidene, or indanyl;
heterocyclyl is 1H-indolyl, 1H-pyrrolyl, 2,3-dihydro-benzo[1,4]dioxinyl, 2H-benzopyranyl, 2-oxo-benzopyranyl, 2-oxo-pyrrolidinyl, 4-oxo-3,4-dihydro-phthalazinyl, 4-oxo-benzopyranyl, 9H-xanthenyl, benzo[1,3]dioxolyl, benzo[2,1,3]oxadiazolyl, benzo[1,2,5]oxadiazolyl, benzo[b]thienyl, cinnolyl, furyl, imidazolyl, morpholinyl, oxazolyl, pyrazolyl, pyridyl, pyrimidyl, pyrrolidinyl, quinoxalyl, thiazolyl, or thienyl; and
halogen is fluoro, chloro, bromo, or iodo;
or a pharmaceutically acceptable salt, hydrate, or solvate thereof.

5. The compound according to claim 4 wherein R₁ is selected from the group consisting of:
(i) C₁₋₅ alkyl substituted by substituent(s) independently selected from the group consisting of:
carbocyclic aryloxy,
carbocyclic aryloxy substituted by halogen, and
carbocyclic aryl,
(ii) C₃₋₆ cycloalkyl substituted by substituent(s) independently selected from the group consisting of:
carbocyclic aryloxy, and
carbocyclic aryloxy substituted by halogen,
(iii) carbocyclic aryl, and
carbocyclic aryl substituted by substituent(s) independently selected from the group consisting of:
halogen,
nitro,
C₁₋₅ alkyl,
C₁₋₅ alkyl substituted by halogen,
C₁₋₅ alkoxy,
C₁₋₅ alkoxy substituted by halogen,
mono-C₁₋₅ alkylaminosulfonyl,
di-C₁₋₅ alkylaminosulfonyl,
carbocyclic aryloxy, and
carbocyclic aryloxy substituted by C₁₋₅ alkoxy,
(iv) heterocyclyl,
heterocyclyl substituted by halogen, and
heterocyclyl substituted by carbocyclic aryloxy;
R₂ and R₃ are each hydrogen; A is a single bond; and B is a single bond or —CH₂—;
Z₁ and Z₂ are each independently hydrogen, C₁₋₅ alkyl, or —N(R₄ₐ)(R_{4b}); Z₃ is hydrogen, C₁₋₅ alkyl, C₁₋₅ alkoxy, —N(R₄ₐ)(R_{4b}), or pyrrolidinyl; wherein R₄ₐ and R_{4b} are each independently hydrogen, C₁₋₅ alkyl, or C₁₋₅ alkyl substituted by carbocyclic aryl; Z₄ is hydrogen or C₁₋₅ alkyl;
or
Z₁ and Z₂ are bonded to each other to form a ring and -Z₁-Z₂- is —(CH₂)ₘ—; wherein m is 3 or 4; and
Y is —C(O)—, —C(S)NH—, —C(O)O—, or —CH₂—;
wherein carbocyclic aryl is phenyl or naphthyl;
heterocyclyl is pyridyl, pyrrolidinyl, benzo[2,1,3]oxadiazolyl, or benzo[1,2,5]oxadiazolyl; and
halogen is fluoro, chloro, or bromo;
or a pharmaceutically acceptable salt, hydrate, or solvate thereof.

6. The compound according to claim 5 wherein R₁ is selected from the group consisting of:
(i) C₁₋₅ alkyl substituted by substituent(s) independently selected from the group consisting of:
carbocyclic aryloxy, and
carbocyclic aryloxy substituted by halogen,
(ii) C₃₋₆ cycloalkyl substituted by substituent(s) independently selected from the group consisting of:
carbocyclic aryl, and
carbocyclic aryl substituted by halogen,
(iii) carbocyclic aryl, and
carbocyclic aryl substituted by substituent(s) independently selected from the group consisting of:
halogen,
nitro,
C₁₋₅ alkyl,
C₁₋₅ alkyl substituted by halogen,
C₁₋₅ alkoxy,
C₁₋₅ alkoxy substituted by halogen,
carbocyclic aryloxy, and
carbocyclic aryloxy substituted by C₁₋₅ alkoxy,
(iv) heterocyclyl, and
heterocyclyl substituted by halogen;
R₂ and R₃ are each hydrogen; A is a single bond; and B is a single bond or —CH₂—;
Z₁ and Z₂ are each independently hydrogen, C₁₋₅ alkyl, or —N(R₄ₐ)(R_{4b}); Z₃ is hydrogen, C₁₋₅ alkyl, or —N(R₄ₐ)(R_{4b}), wherein R₄ₐ and R_{4b} are each independently hydrogen or C₁₋₅ alkyl; Z₄ is hydrogen;
or
Z₁ and Z₂ are bonded to each other to form a ring and -Z₁-Z₂- is —(CH₂)ₘ—; wherein m is 3 or 4; and
Y is —C(O)—;
wherein carbocyclic aryl is phenyl;
heterocyclyl is pyridyl, benzo[2,1,3]oxadiazolyl, or benzo[1,2,5]oxadiazolyl; and
halogen is fluoro, chloro, or bromo;
or a pharmaceutically acceptable salt, hydrate, or solvate thereof.

7. The compound according to claim 1 selected from the group consisting of:
3-chloro-4-fluoro-N-{cis-4-[(4-methoxypyridin-2-yl)amino]cyclohexyl}benzamide;
N²-{cis-4-[(3-chloro-4-fluorobenzyl)amino]cyclohexyl}-N⁴,N⁴,5-trimethylpyridine-2,4-diamine;
N-{cis-4-[(4-amino-5-methylpyridin-2-yl)amino]cyclohexyl}-3,4,5-trifluorobenzamide;
4-bromophenyl (cis-4-{[4-(dimethylamino)-5-methylpyridin-2-yl]amino}cyclohexyl)carbamate;
3-chloro-4-fluoro-N-{cis-4-[(4-methylpyridin-2-yl)amino]cyclohexyl}benzamide;
3-chloro-4-fluoro-N-[cis-4-({5-methyl-4-[methyl(2-phenylethyl)amino]pyridin-2-yl}amino)cyclohexyl]benzamide;
3-chloro-4-fluoro-N-{cis-4-[(6-methylpyridin-2-yl)amino]cyclohexyl}benzamide;
N-{cis-4-[(4-amino-5-methylpyridin-2-yl)amino]cyclohexyl}-3-chloro-4-fluorobenzamide;
3-chloro-4-fluoro-N-{cis-4-[(5-methyl-4-pyrrolidin-1-ylpyridin-2-yl)amino]cyclohexyl}benzamide;
3-chloro-4-fluoro-N-{cis-4-[(5-methylpyridin-2-yl)amino]cyclohexyl}benzamide;
N-(3-chloro-4-fluorophenyl)-N′-(cis-4-{[4-(dimethylamino)-5-methylpyridin-2-yl]amino}cyclohexyl)thiourea;
N-(3-chloro-4-fluorophenyl)-N′-(cis-4-{[4-(dimethylamino)-5-methylpyridin-2-yl]amino}cyclohexyl)urea;
3-chloro-4-fluoro-N-{cis-4-[(3,5,6-trimethylpyridin-2-yl)amino]cyclohexyl}benzamide;
3-chloro-N-(cis-4-{[4-(dimethylamino)-6-methylpyridin-2-yl]amino}cyclohexyl)-4-fluorobenzamide;
N-(cis-4-{[4-(dimethylamino)-6-methylpyridin-2-yl]amino}cyclohexyl)-3,4-difluorobenzamide;
N-(cis-4-{[4-(dimethylamino)-6-methylpyridin-2-yl]amino}cyclohexyl)-2-(4-methoxyphenoxy)-5-nitrobenzamide;
N-(cis-4-{[4-(dimethylamino)-6-methylpyridin-2-yl]amino}cyclohexyl)-2,1,3-benzoxadiazole-5-carboxamide;
1-(4-chlorophenyl)-N-(cis-4-{[4-(dimethylamino)-6-methylpyridin-2-yl]amino}cyclohexyl)cyclopentanecarboxamide;
N-(cis-4-{[4-(dimethylamino)-6-methylpyridin-2-yl]amino}cyclohexyl)-3-nitrobenzamide;
2-(4-chlorophenoxy)-N-(cis-4-{[4-(dimethylamino)-6-methylpyridin-2-yl]amino}cyclohexyl)acetamide; and
4-chloro-N-(cis-4-{[4-(dimethylamino)-6-methylpyridin-2-yl]amino}cyclohexyl)benzamide;
or a pharmaceutically acceptable salt, hydrate, or solvate thereof.

8. The compound according to claim 1 selected from the group consisting of:
3-chloro-N—[(cis-4-{[4-(dimethylamino)-5-methylpyridin-2-yl]amino}cyclohexyl)methyl]-4-fluorobenzamide;
3-chloro-N-[cis-4-({4-[ethyl(methyl)amino]-5-methylpyridin-2-yl}amino)cyclohexyl]-4-fluorobenzamide;
3,4,5-trifluoro-N-(cis-4-{[5-methyl-4-(methylamino)pyridin-2-yl]amino}cyclohexyl)benzamide;
N-(cis-4-{[4-(dimethylamino)-5-methylpyridin-2-yl]amino}cyclohexyl)-3-(trifluoromethoxy)benzamide;
5-bromo-N-(cis-4-{[4-(dimethylamino)-5-methylpyridin-2-yl]amino}cyclohexyl)nicotinamide;
3-chloro-N-(cis-4-{[6-(dimethylamino)pyridin-2-yl]amino}cyclohexyl)-4-fluorobenzamide;
3-chloro-4-fluoro-N-(cis-4-{[5-methyl-4-(methylamino)pyridin-2-yl]amino}cyclohexyl)benzamide;
N-(cis-4-{[4-(dimethylamino)-5-methylpyridin-2-yl]amino}cyclohexyl)-4-fluoro-3-(trifluoromethyl)benzamide;
N-(cis-4-{[4-(dimethylamino)-5-methylpyridin-2-yl]amino}cyclohexyl)-3-(trifluoromethyl)benzamide;
3,5-dichloro-N-(cis-4-{[4-(dimethylamino)-5-methylpyridin-2-yl]amino}cyclohexyl)benzamide;
3-chloro-N-(cis-4-{[4-(dimethylamino)-5-methylpyridin-2-yl]amino}cyclohexyl)benzamide;
3,4-dichloro-N-(cis-4-{[4-(dimethylamino)-5-methylpyridin-2-yl]amino}cyclohexyl)benzamide;
3-chloro-N-(cis-4-{[4-(dimethylamino)-5-methylpyridin-2-yl]amino}cyclohexyl)-5-fluorobenzamide;
3,4,5-trifluoro-N-[cis-4-(5,6,7,8-tetrahydroquinolin-2-ylamino)cyclohexyl]benzamide;
3-chloro-4-fluoro-N-[cis-4-(5,6,7,8-tetrahydroquinolin-2-ylamino)cyclohexyl]benzamide;
N-(cis-4-{[4-(dimethylamino)pyridin-2-yl]amino}cyclohexyl)-3,4,5-trifluorobenzamide;
N-(cis-4-{[4-(dimethylamino)-5-methylpyridin-2-yl]amino}cyclohexyl)-3,4-difluorobenzamide;
3-chloro-N-(cis-4-{[4-(dimethylamino)pyridin-2-yl]amino}cyclohexyl)-4-fluorobenzamide;
3-chloro-N-{cis-4-[(5,6-dimethylpyridin-2-yl)amino]cyclohexyl}-4-fluorobenzamide;
N-(cis-4-{[4-(dimethylamino)-6-methylpyridin-2-yl]amino}cyclohexyl)-3,4,5-trifluorobenzamide;
N-(cis-4-{[4-(dimethylamino)-5-methylpyridin-2-yl]amino}cyclohexyl)-3,4,5-trifluorobenzamide;
3-chloro-N-(cis-4-{[4-(dimethylamino)-6-methylpyridin-2-yl]amino}cyclohexyl)-4-fluorobenzamide;
3-chloro-4-fluoro-N-{cis-4-[(4-methyl-5,6,7,8-tetrahydroquinolin-2-yl)amino]cyclohexyl}benzamide;
3-chloro-4-fluoro-N-{cis-4-[(4-methyl-6,7-dihydro-5H-cyclopenta[b]pyridin-2-yl)amino]cyclohexyl}benzamide;
3-chloro-N-(cis-4-{[4-(dimethylamino)-5-methylpyridin-2-yl]amino}cyclohexyl)-4-fluorobenzamide;
3-chloro-4-fluoro-N-{cis-4-[(4,5,6-trimethylpyridin-2-yl)amino]cyclohexyl}benzamide;
3-chloro-N-{cis-4-[(4,5-dimethylpyridin-2-yl)amino]cyclohexyl}-4-fluorobenzamide;
3-chloro-N-{cis-4-[(4,6-dimethylpyridin-2-yl)amino]cyclohexyl}-4-fluorobenzamide;
N-(cis-4-{[2-(4-chlorophenoxy)ethyl]amino}cyclohexyl)-N,N-6-trimethylpyridine-2,4-diamine;
N-(cis-4-{[4-(dimethylamino)-6-methylpyridin-2-yl]amino}cyclohexyl)-2-(1-naphthyl)acetamide;
N-(cis-4-{[4-(dimethylamino)-6-methylpyridin-2-yl]amino}cyclohexyl)-3-(trifluoromethyl)benzamide;
N-(cis-4-{[4-(dimethylamino)-6-methylpyridin-2-yl]amino}cyclohexyl)-4-[(dipropylamino)sulfonyl]benzamide; and
N-(cis-4-{[4-(dimethylamino)-6-methylpyridin-2-yl]amino}cyclohexyl)-2-phenoxynicotinamide;
or a pharmaceutically acceptable salt, hydrate, or solvate thereof.

9. The compound according to claim 1 wherein R₁ is selected from hydrogen or —CO₂Bn (Bn is a benzyl group); R₂ and R₃ are each hydrogen; A and B are each independently a single bond or —CH₂—, provided that A is not —CH₂— when B is —CH₂—; Z₁ and Z₂ are each independently hydrogen, halogen, C₁₋₅ alkyl, or —N(R₄ₐ)(R_{4b}); Z₃ is hydrogen, cyano, nitro, carbamoyl, C₁₋₅ alkyl, C₁₋₅ alkyl substituted by hydroxy, C₁₋₅ alkoxy, —C(O)N(R₄ₐ)(R_{4b}), —N(R₄ₐ)(R_{4b}), morpholinyl, pyrrolidinyl, or imidazolyl; wherein R₄ₐ and R_{4b} are each independently hydrogen, C₁₋₅ alkyl, or C₁₋₅ alkyl substituted by carbocyclic aryl; Z₄ is hydrogen, halogen, or C₁₋₅ alkyl; or Z₁ and Z₂ are bonded to each other to form a ring and -Z₁-Z₂- is —(CH₂)ₘ—; wherein m is 3 or 4; Y is a single bond; and q is 0 or 1; or a pharmaceutically acceptable salt, hydrate, or solvate thereof.

10. The compound according to claim 9 wherein R₁ is selected from hydrogen or —CO₂Bn (Bn is a benzyl group); R₂ and R₃ are each hydrogen; A is a single bond; B is a single bond or —CH₂—; Z₁ and Z₂ are each independently hydrogen, C₁₋₅ alkyl, or —N(R₄ₐ)(R_{4b}); Z₃ is hydrogen, C₁₋₅ alkyl, or —N(R₄ₐ)(R_{4b}); wherein R₄ₐ and R_{4b} are each independently hydrogen or C₁₋₅ alkyl; Z₄ is hydrogen; or Z₁ and Z₂ are bonded to each other to form a ring and -Z₁-Z₂- is —(CH₂)ₘ—; wherein m is 3 or 4; and Y is a single bond; or a pharmaceutically acceptable salt, hydrate, or solvate thereof.

11. A pharmaceutical composition comprising a therapeutically effective amount of a compound according to claim 1 in combination with a pharmaceutically acceptable carrier.

12. A method for the prophylaxis or treatment of improving memory function, sleeping and arousal, anxiety, depression, mood disorders, seizure, obesity, diabetes, appetite and eating disorders, cardiovascular disease, hypertension, dyslipidemia, myocardial infarction, binge eating disorders including bulimia, anorexia, mental disorders including manic depression, schizophrenia, delirium, dementia, stress, cognitive disorders, attention deficit disorder, substance abuse disorders and dyskinesias including Parkinson's disease, epilepsy, and addiction comprising administering to an individual suffering from said condition a therapeutically effective amount of a compound according to claim 1.

13. A method for the prophylaxis or treatment of an eating disorder, obesity or an obesity related disorder comprising administering to an individual suffering from said condition a therapeutically effective amount of a compound according to claim 1.

14. A method for the prophylaxis or treatment of anxiety, depression, schizophrenia, addiction, or epilepsy comprising administering to an individual suffering from said condition a therapeutically effective amount of a compound according to claim 1.

15. A compound according to claim 1 for use in a method of treatment of the human or animal body by therapy.

16. A compound according to claim 1 for use in a method of prophylaxis or treatment of an eating disorder, obesity or an obesity related disorder of the human or animal body by therapy.

17. A compound according to claim 1 for use in a method of prophylaxis or treatment of anxiety, depression, schizophrenia, addiction, or epilepsy of the human or animal body by therapy.

18. A compound according to claim 1 for the manufacture of a medicament for use in the prophylaxis or treatment of an eating disorder, obesity or obesity related disorders.

19. A compound according to claim 1 for the manufacture of a medicament for use in the prophylaxis or treatment of anxiety, depression, schizophrenia, addiction, or epilepsy.

20. A method of decreasing food intake of an individual comprising administering to said individual a therapeutically effective amount of a compound according to claim 1.

21. A method of inducing satiety in an individual comprising administering to said individual a therapeutically effective amount of a compound according to claim 1.

22. A method of controlling or reducing weight gain in an individual comprising administering to said individual a therapeutically effective amount of a compound according to claim 1.

23. A method of modulating a MCH receptor in an individual comprising contacting the receptor with a compound according to claim 1.

24. The method of modulating the MCH receptor according to claim 23 wherein the compound is an antagonist.

25. The method of modulating the MCH receptor according to claim 23 wherein the modulation of the MCH receptor is for the prophylaxis or treatment of an eating disorder, obesity or obesity related disorder.

26. The method of modulating the MCH receptor according to claim 23 wherein the modulation of the MCH receptor reduces food intake of the individual.

27. The method of modulating the MCH receptor according to claim 23 wherein the modulation of the MCH receptor induces satiety in the individual.

28. The method of modulating the MCH receptor according to claim 23 wherein the modulation of the MCH receptor controls or reduces weight gain of the individual.

29. The method of modulating the MCH receptor according to claim 23 wherein the modulation of the MCH receptor is for prophylaxis or treatment of anxiety, depression, schizophrenia, addiction, or epilepsy.

30. The method of modulating the MCH receptor according to claim 13 wherein the individual is a mammal.

31. The method of modulating the MCH receptor according to claim 30 wherein the mammal is a human.

32. The method according to claim 31 wherein the human has a body mass index of about 18.5 to about 45.

33. The method according to claim 32 wherein the human has a body mass index of about 25 to about 45.

34. The method according to claim 33 wherein the human has a body mass index of about 30 to about 45.

35. The method according to claim 34 wherein the human has a body mass index of about 35 to about 45.

36. A method of producing a pharmaceutical composition comprising admixing a compound according to claim 1 and a pharmaceutically acceptable carrier.
